(19) United States
(12) Patent Application Publication
(10) Pub. No.: US 2021/0380623 A1
(43) Pub. Date: Dec. 9, 2021

(54) PRODRUG GALACTOSIDE INHIBITOR OF GALECTINS

(71) Applicant: GALECTO BIOTECH AB, COPENHAGEN (DK)

(72) Inventors: Fredrik ZETTERBERG, Askim (SE); Ulf NILSSON, Lund (SE)

(73) Assignee: GALECTO BIOTECH AB, COPENHAGEN (DK)

(21) Appl. No.: 17/285,225

(22) PCT Filed: Oct. 9, 2019
(86) PCT No.: PCT/EP2019/077401

§ 371 Date: Apr. 14, 2021

(30) Foreign Application Priority Data
Oct. 15, 2018 (EP) 18200402.8

(51) Int. Cl. C07H 19/056

(52) U.S. Cl. CPC C07H 19/056

(57) ABSTRACT

A prodrug compound of the general formula I or II. The prodrug compound of formula I or II is suitable for use in a method for treating a disorder relating to the binding of a galectin, such as galectin-3 to a ligand in a mammal, such as a human. Also, a method for treatment of a disorder relating to the binding of a galectin, such as galectin-3 to a ligand in a mammal, such as a human.

### TECHNICAL FIELD

The present invention relates to novel prodrug compounds, the use of said compounds as medicament and for the manufacture of a medicament for the treatment of inflammation; fibrosis; scarring; keloid formation; aberrant scar formation; surgical adhesions; septic shock; cancers; autoimmune diseases; metabolic disorders; heart disease; heart failure; pathological angiogenesis; eye diseases; atherosclerosis; metabolic diseases; asthma and other interstitial lung diseases; and liver disorders in mammals. The invention also relates to pharmaceutical compositions comprising said novel prodrug compounds.

### BACKGROUND ART

Galectins are proteins with a characteristic carbohydrate recognition domain (CRD) (Leffler et al., 2004). This is a tightly folded β-sandwich of about 130 amino acids (about 15 kDa) with the two defining features 1) a β-galactose binding site and 2) sufficient similarity in a sequence motif of about seven amino acids, most of which (about six residues) make up the β-galactose binding site. However, sites adjacent to the β-galactose site are required for tight binding of natural saccharides and different preferences of these give galectins different fine specificity for natural saccharides.

The recent completion of the human, mouse and rat genome sequences reveal about 15 galectins and galectin-like proteins in one mammalian genome with slight variation between species (Leffler et al., 2004).

Galectin subunits can contain either one or two CRDs within a single peptide chain. The first category, mono-CRDs galectins, can occur as monomers or dimers (two types) in vertebrates. The by far best studied galectins are the dimeric galectin-1, and galectin-3 that is a monomer in solution but may aggregate and become multimeric upon encounter with ligands (Lepur et al., 2012). These were the first discovered galectins and are abundant in many tissues.

There are now over 5700 publications on galectins in PubMed, with most, as mentioned above, about galectins-1 (>1400) and -3 (>2800). Strong evidence suggests roles for galectins in e.g. inflammation and cancer, and development (Blidner et al., 2015, Ebrahim et al., 2014).

Galectins are synthesized as cytosolic proteins, without a signal peptide on free ribosomes. Their N-terminus is acetylated, a typical modification of cytosolic proteins, and they reside in the cytosol for a long time (not typical of secreted proteins). From there they can be targeted to the nucleus, specific cytososlic sites, or secreted (induced or constitutively) by a non-classical (non-ER-Golgi) pathway, as yet unknown, but possibly similar to the export of e.g. IL-1 (Leffler et al., 2004; Arthur et al., 2015). They can also function in all these compartments; for galectin-3, solid evidence published in well respected journals support roles in RNA splicing in the nucleus, inhibition of apoptosis in the cytosol, accumulation around disrupted vesicles, association with microtubule organizing center of cilia, and a variety of extracellular effects on cell signaling and adhesion (Elola et al. 2015, Funasaka et al., 2014, Aits et al., 2015, Clare et al., 2014). Other galectins also may act in the cytosol by enhancing apoptosis and regulating the cell cycle and differentiation in certain cells. Most galectins act also extracellularly by cross-linking glycoproteins (e.g. laminin, integrins, and IgE receptors) possibly forming supramolecular ordered arrays (Elola et al., 2015) and may thereby modulate cell adhesion and induce intracellular signals. Related to this, recent years have seen the emergence of a molecular mechanism of these galectin functions involving a formation of microdomains (lattices) within membranes, (Elola et al., 2015) which in turn affects intracellular trafficking and cell surface presentation of glycoprotein receptors. This has been documented in cell culture, in null mutant mice, and animals treated with galectin or galectin inhibitors (Johannes, L.; Jacob, R.; Leffler, H. Galectins at a Glance. J. Cell. Sci. 2018, 131 (9), jcs208884.).

### Potential Therapeutic Use of Galectin-3 Inhibitors

Galectin-3 has been implicated in diverse phenomena and, hence, inhibitors may have multiple uses (Blanchard et al., 2014). It is easy to perceive this as a lack of specificity or lack of scientific focus. Therefore, the analogy with aspirin and the cyclooxygenases (COX-I and II) is useful. The COXs produce the precursor of a wide variety of prostaglandins and, hence, are involved in a diverse array of biological mechanisms. Their inhibitors, aspirin and other NSAIDs (non-steroid anti-inflammatory drugs), also have broad and diverse effects. Despite this, these inhibitors are very useful medically, and they have several different specific utilities.

So if galectins, like COXs, are part of some basic biological regulatory mechanism (as yet unknown), they are likely to be ‘used by nature’ for different purpose in different contexts. Galectin inhibitors, like NSAIDs, are not expected to wipe out the whole system, but to tilt the balance a bit.

### Inhibition of Inflammation

A pro-inflammatory role of galectin-3 is indicated by its induction in cells at inflammatory sites, a variety of effects on immune cells (e.g. oxidative burst in neutrophils and chemotaxis in monocytes), and decrease of the inflammatory response, mainly in neutrophils and macrophages, in null mutant mice (Blidner et al., 2015, Arthur et al., 2015). Importantly, recent studies have identified galectin-3 as a key rate-limiting factor in macrophage M2 differentiation and myofibroblast activation, which influences the development of fibrosis (Mackinnon et al., 2008; Mackinnon et al., 2012, Li et al., 2014).

Inflammation is a protective response of the body to invading organisms and tissue injury. However, if unbalanced, frequently it is also destructive and occurs as part of the pathology in many diseases. Because of this, there is great medical interest in pharmacological modulation of inflammation. A galectin-3 inhibitor is expected to provide an important addition to the arsenal available for this.

### Treatment of Fibrosis-Related Conditions

The idea of a possible role of galectin-3 in fibrosis comes from cell and ex vivo studies on macrophage differentiation (Mackinnon et al., 2008), as well as from in vivo studies on macrophage differentiation and myofibroblast activation (Mackinnon et al., 2012). Briefly, the hypothesis is as follows: Galectin-3 has been shown to prolong cell surface residence and thus enhance responsiveness of certain receptors (Elola et al., 2015), such as the TGF-β receptor (MacKinnon, 2012), which in turn regulates alternative macrophage differentiation into M2 macrophages and myofibroblast activation.

Hence, as galectin-3 is a good candidate for being an endogenous enhancer of TGF-β signaling and alternative macrophage differentiation and myofibroblast activation, galectin-3 inhibitors may be very useful in treating fibrosis and adverse tissue remodeling.

### Treatment of Cancer

A large number of immunohistochemical studies show changed expression of certain galectins in cancer (Thijssen et al, 2015; Ebrahim et al., 2014) and for example galectin-3 is now an established histochemical marker of thyroid cancer. The direct evidence for a role of galectin-3 in cancer comes mainly from mouse models. In paired tumor cell lines (with decreased or increased expression of galectin-3), the induction of galectin-3 gives more tumors and metastasis and suppression of galectin-3 gives less tumors and metastasis. Galectin-3 has been proposed to enhance tumor growth by being anti-apoptotic, promote angiogenesis, or to promote metastasis by affecting cell adhesion. Further, recent evidence have shown that galectin-3 plays a critical role in the tumor microenvironment (Ruvolo, 2015). Galectin-3 is also believed to regulate the interaction between the tumor cells and immune cells, such as T-lymphocytes (T-cells), and inhibition of galectin-3 has been shown to restore T-cell activity (Demotte et al. 2010, Kouo et al. 2015, Menero et al. 2015). From the above it is clear that inhibitors of galectin-3 might have valuable anti-cancer effects. Indeed, saccharides claimed but not proven to inhibit galectin-3 have been reported to have anti-cancer effects. In our own study a fragment of galectin-3 containing the CRD inhibited breast cancer in a mouse model by acting as a dominant negative inhibitor (John et al., 2003). More recently, inhibition of galectin-3 with small molecules have been demonstrated to indeed greatly enhance tumor cell sensitivity towards radiation and standard pro-apoptotic drugs in cell assays and ex vivo (Blanchard et al., 2015).

Also other galectins are frequently over-expressed in low differentiated cancer cells, or induced in specific cancer types (Thijssen et al, 2015; Ebrahim et al., 2014). Galectin-1 induces apoptosis in activated T-cells and has a remarkable immunosuppressive effect on autoimmune disease in vivo (Blidner et al., 2015). Therefore, the over-expression of these galectins in cancers might help the tumor to defend itself against the T-cell response raised by the host.

Null mutant mice for galectins-1, -3, -7 and -9 have been established and are healthy and reproduce apparently normally in animal house conditions. However, further studies have revealed subtle phenotypes under different type of challenge, mainly in function of immune cells (Blidner et al., 2015), but also other cells types (Viguier et al., 2014). The differences in site of expression, specificity and other properties make it unlikely that different galectins can replace each other functionally. The observations in the null mutant mice would indicate that galectins are not essential for basic life supporting functions as can be observed in normal animal house conditions. Instead they may be optimizers of normal function and/or essential in stress conditions not found in animal house conditions. The lack of strong effect in null mutant mice may make galectin inhibitors more favorable as drugs. If galectin activity contributes to pathological conditions as suggested above but less to normal conditions, then inhibition of them will have less unwanted side effects.

### Treatment of Angiogenesis

Vascular endothelial growth factors (VEGFs) signaling through VEGF receptor-2 (VEGFR-2) is the primary angiogenic pathway. Studies have been published demonstrating that both galectin-1 (Gal-1) and galectin-3 (Gal-3) are important modulators for VEGF/VEGFR-2 signaling pathway (Croci et al., 2014). It has also been published that a galectin inhibitor, TDX, is expected have efficacy against pathological angiogenesis. (Chen 2012)

### Known Inhibitors

### Natural Ligands

Solid phase binding assays and inhibition assays have identified a number of saccharides and glycoconjugates with the ability to bind galectins (reviewed by Leffler, 2001 and Leffler et al., 2004). All galectins bind lactose with a K_{d} of 0.5-1 mM. The affinity of D-galactose is 50-100 times lower. N-Acetyllactosamine and related disaccharides bind about as well as lactose, but for certain galectins, they can bind either worse or up to 10 times better. The best small saccharide ligands for galectin-3 were those carrying blood group A-determinants attached to lactose or LacNAc-residues and were found to bind up to about 50 times better than lactose. Galectin-1 shows no preference for these saccharides.

Larger saccharides of the polylactosamine type have been proposed as preferred ligands for galectins. In solution, using polylactosamine-carrying glycopeptides, there was evidence for this for galectin-3, but not galectin-1 (Leffler and Barondes, 1986). A modified plant pectin polysaccharide has been reported to bind galectin-3 (Pienta et al., 1995).

The above-described natural saccharides that have been identified as galectin-3 ligands are not suitable for use as active components in pharmaceutical compositions, because they are susceptible to acidic hydrolysis in the stomach and to enzymatic degradation. In addition, natural saccharides are hydrophilic in nature, and are not readily absorbed from the gastrointestinal tract following oral administration.

### Galectin Specificity

The studies of galectin specificity using inhibition by small natural saccharides mentioned above indicated that all galectins bound lactose, LacNAc and related disaccharides, but that galectin-3 bound certain longer saccharides much better (Leffler and Barondes, 1986). These longer saccharides were characterized by having an additional sugar residue added to the C-3 position of galactose (in e.g. lactose or LacNAc) that bound an extended binding groove. The shape of this groove varies between galectins, suggesting that the same extensions would not be bound equally by the different galectins.

### Synthetic Inhibitors

Saccharides coupled to amino acids with anti-cancer activity were first identified as natural compounds in serum, but subsequently, synthetic analogues have been made (Glinsky et al., 1996). Among them, those with lactose or galactose coupled to the amino acid inhibit galectins, but only with about the same potency as the corresponding underivatized sugar. A chemically modified form of citrus pectin (Platt and Raz, 1992) that inhibits galectin-3 shows anti-tumor activity in vivo (Pienta et al., 1995; Nangia-Makker et al., 2002).

Cluster molecules having up to four lactose moieties showed a strong multivalency effect when binding to galectin-3, but not to galectin-1 and galectin-5 (Vrasidas et al., 2003). Cyclodextrin-based glycoclusters with seven galactose, lactose, or N-acetyllactosamine residues also showed a strong multivalency effect against galectin-3, but less so against galectins-1 and -7 (André et al., 2004). Starburst dendrimers (André et al., 1999) and glycopolymers (Pohl et al., 1999; David et al., 2004), made polyvalent in lactose-residues, have been described as galectin-3 inhibitors with marginally improved potency as compared to lactose. The aforementioned synthetic compounds that have been identified as galectin-3 ligands are not suitable for use as active components in pharmaceutical compositions, because they are hydrophilic in nature and are not readily absorbed from the gastrointestinal tract following oral administration.

Natural oligosaccharides, glycoclusters, glycodendrimers, and glycopolymers described above are too polar and too large to be absorbed and in some cases are large enough to produce immune responses in patients. Furthermore, they are susceptible to acidic hydrolysis in the stomach and to enzymatic hydrolysis. Thus, there is a need for small synthetic molecules.

Thiodigalactoside is known to be a synthetic and hydrolytically stable, yet polar inhibitor, approximately as efficient as N-acetyllactosamine (Leffler and Barondes, 1986). N-Acetyllactosamine derivatives carrying aromatic amides or substituted benzyl ethers at C-3′ have been demonstrated to be highly efficient inhibitors of galectin-3, with unprecedented IC₅₀ values as low as 4.8 μM, which is a 20-fold improvement in comparison with the natural N-acetyllactosamine disaccharide (Sörme et al., 2002; Sörme et al., 2003b). These derivatives are less polar overall, due to the presence of the aromatic amido moieties and are thus more suitable as agents for the inhibition of galectins in vivo. Furthermore, C3-triazolyl galactosides have been demonstrated to be as potent inhibitors as the corresponding C3-amides of some galectins. Hence, any properly structured galactose C3-substituent may confer enhanced galectin affinity.

However, the C3-amido- and C3-triazolyl-derivatised compounds are still susceptible to hydrolytic degradation in vivo, due to the presence of a glycosidic bond in the galactose and N-acetyllactosamine saccharide moiety and, although they are potent small molecule inhibitors of galectin-3, even further improved affinity and stability is desirable. Accordingly, inhibitors based on 3,3′-diamido- or 3,3′-ditriazolyl-derivatization of thiodigalactoside have been developed, (Cumpstey et al., 2005b; Cumpstey et al., 2008; Salameh et al., 2010; WO/2005/113569 and US2007185041; WO/2005/113568, U.S. Pat. No. 7,638,623 B2, T. Delaine, 2016, ChemBioChem 10.1002/cbic.201600285)) which lack O-glycosidic hydrolytically and enzymatically labile linkages. These inhibitors also displayed superior affinity for several galectins (down to Kd in the low nM range). Nevertheless, although displaying high affinity for galectins, the 3,3′-derivatized thiodigalactosides still comprise a disadvantage in their multistep synthesis involving double inversion reaction to reach at 3-N-derivatized galactose building blocks. Furthermore, cyclohexane replacement of one galactose ring in thiodigalactoside has been evidenced to mimic the galactose ring and hence to provide galectin-1 and -3 inhibitors with efficiency approaching those of the diamido- and ditriazolyl-thiodigalactoside derivatives (WO/2010/126435). Replacement of a D-galactopyranose unit with a substituted cyclohexane decreases polarity and most likely also metabolic susceptibility, thus improving drug-like properties.

Some earlier described compounds have the following general formulas

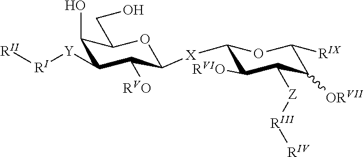

as described in WO/2005/113568,

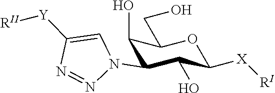

and
as described in WO/2005/113569, in which R¹ can be a D-galactose.

In recently published US20140099319, WO2014067986 and (T. Delaine, 2016, ChemBioChem 10.1002/cbic.201600285) are disclosed a compound of formula

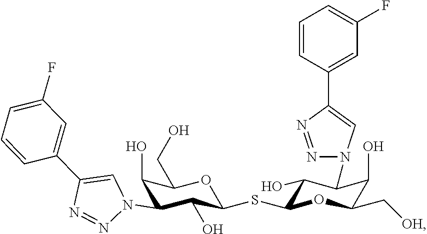

having fluorine (F) in the meta position on both the phenyl rings in relation to the triazole rings. This compound has been shown to be a promising drug candidate for lung fibrosis, and in particular is very selective on galectin-3 with high affinity.

A series of small C1 or C1 and C3-substituted galactopyranosides have been disclosed showing affinity towards galectin-3 and 1. The beta-D-galactopyranosides were reported as having affinity in the same range or less than lactose, which has a Kd of about 91 μM towards galectin-3 and 190 μM towards galectin-1. (Giguere, D et. al. 2011, 2008, 2006).

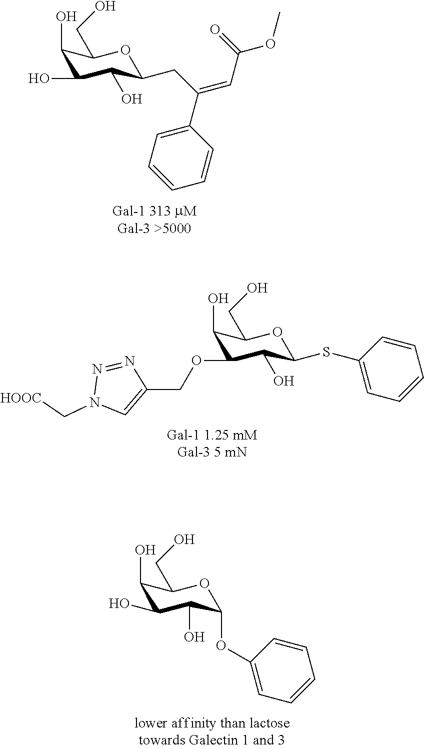

There is no disclosure or mentioning of corresponding alpha-anomers having affinity towards galectin-3 or galectin-1 better than lactose.

### SUMMARY OF THE INVENTION

The compounds of the present invention are novel prodrugs of galactopyranose compounds that unexpectedly have good solubility and can be used to increase the maximum dose resulting in dose correlated bioavailability. The prodrugs of the present invention convert into an active metabolite which has high affinity to galectin 3 and inhibits galectin 3. Here prodrugs have been developed in which one to three functional group(s) are introduced at selected positions to prepare a prodrug which is charged in the intestine of a mammal, such as a human, due to it's pKa.

In broad aspect the present invention concerns a prodrug compound of formula (I)

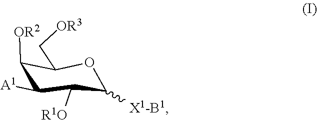

wherein the pyranose ring is α- or β-D-galactopyranose (as indicated by wavy line);
wherein:

A¹ is selected from the group consisting of i) an aryl; ii) an aryl substituted with at least one from the group consisting of a halogen; CN; C₂₋₆ alkenyl; C₂₋₆ alkynyl; carboxyl; C₁₋₆ alkoxy; C₁₋₆ thio alkyl; C₁₋₆ alkyl; nitro; thio; C₁₋₆ alkylthio; amino; hydroxy; C₁₋₆ carbonyl; an amino; and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; iii) a C₁₋₆ alkoxy; iv) a C₁₋₆ alkoxy substituted with at least one from the group consisting of a halogen; a C₁₋₆ alkyl; a heteroaryl; a heteroaryl substituted with at least one from the group consisting of halogen, CN, C₂₋₆ alkenyl, C₂₋₆ alkynyl, carboxyl, C₁₋₆ alkoxy, C₁₋₆ thioalkyl, amino, C₁₋₆ alkyl, C₁₋₆ alkyl substituted with at least one halogen, C₁₋₆ alkoxy, C₁₋₆ alkoxy substituted with at least one halogen, a five or six membered heteroaromatic ring, a five or six membered heteroaromatic ring substituted with at least one from the group consisting of halogen, CN, C₂₋₆ alkenyl, C₂₋₆ alkynyl, carboxyl, C₁₋₆ alkoxy, C₁₋₆ thioalkyl, C₁₋₆ alkyl, C₁₋₆ alkyl substituted with at least one halogen, C₁₋₆ alkoxy, and C₁₋₆ alkoxy substituted with at least one halogen, an aryl, and an aryl substituted with at least one from the group consisting of halogen, CN, C₂₋₆ alkenyl, C₂₋₆ alkynyl, carboxyl, C₁₋₆ alkoxy, C₁₋₆ thioalkyl, C₁₋₆ alkyl, C₁₋₆ alkyl substituted with at least one halogen, C₁₋₆ alkoxy, and C₁₋₆ alkoxy substituted with at least one halogen; an amino; and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; v) a C₁₋₆ alkylamino; vi) a C₁₋₆ alkylamino substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; vii) a heteroaryl; viii) a heteroaryl substituted with at least one from the group consisting of a halogen; CN; C₂₋₆ alkenyl; C₂₋₆ alkynyl; carboxyl; C₁₋₆ alkoxy; C₁₋₆ thioalkyl; an amino; an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; an aryl; an aryl substituted with at least one from the group consisting of a halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a heteroaryl; a heteroaryl substituted with at least one from the group consisting of halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₃₋₇ cycloalkyl, C₃₋₇ cycloalkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₃₋₇ cycloalkoxy, and C₃₋₇ cycloalkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a C₁₋₆ carbonyl; a C₁₋₆ carbonyl substituted with at least one from the group consisting of halogen, C₁₋₆ alkoxy, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, and an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; ix) a heterocycle; x) a heterocycle substituted with at least one from the group consisting of halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₃₋₇ cycloalkyl, C₃₋₇ cycloalkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₃₋₇ cycloalkoxy, and C₃₋₇ cycloalkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a C₁₋₆ carbonyl; a C₁₋₆ carbonyl substituted with at least one from the group consisting of halogen, C₁₋₆ alkoxy, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, and an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; xi) a C₁₋₆ alkyl; xii) a C₁₋₆ alkyl substituted with at least one from the group consisting of halogen; C₁₋₆ alkoxy; C₁₋₆ alkyl; C₃₋₇ cycloalkyl; nitro; thio; C₁₋₆ alkylthio; amino; hydroxy; and C₁₋₆ carbonyl; xiii) a C₁₋₆ carbonyl; xiv) a C₁₋₆ carbonyl substituted with at least one from the group consisting of a C₁₋₆ alkyl; a C₂₋₆ alkenyl; an aryl; a heteroaryl; and a heterocycle; xv) a C₁₋₆ alkyl-CONH—; xvi) a C₁₋₆ alkyl-CONH— substituted on one or more alkyl carbon with at least one from the group consisting of a heteroaryl; a heteroaryl substituted with at least one from the group consisting of a halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; nitro, thio, C₁₋₆ alkylthio, amino, hydroxy, C₁₋₆ carbonyl, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; an aryl; and an aryl substituted with at least one from the group consisting of a halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; or an in vivo metabolizable group of A¹;

X¹ is selected from the group consisting of O, S, SO, SO₂, C═O, amino, amino substituted with a C₁₋₆ alkyl, and CR′R″ wherein R′ and R″ are independently selected from hydrogen, OH, or halogen; or an in vivo metabolizable group of X¹;

B¹ is selected from the group consisting of a) a C₁₋₆ alkyl, b) a C₁₋₆ alkyl substituted with at least one from the group consisting of a five or six membered heteroaromatic ring; a five or six membered heteroaromatic ring substituted with at least one from the group consisting of cyano, halogen, C₁₋₆ alkyl, C₁₋₆ alkyl substituted with at least one from the group consisting of halogen, hydroxy and C₁₋₆ alkyl, C₁₋₆ alkoxy, C₁₋₆ alkoxy substituted with at least one from the group consisting of halogen, hydroxy and C₁₋₆ alkyl, hydroxy, and R^{#}—CONH— wherein R^{#} is selected from the group consisting C₁₋₆ alkyl and C₁₋₆ cycloalkyl; an aryl; and an aryl substituted with at lest one from the group consisting of cyano, halogen, C₁₋₆ alkyl, C₁₋₆ alkyl substituted with at least one from the group consisting of halogen, hydroxy and C₁₋₆ alkyl, C₁₋₆ alkoxy, C₁₋₆ alkoxy substituted with at least one from the group consisting of halogen, hydroxy and C₁₋₆ alkyl, hydroxy, and R^{#}—CONH— wherein R^{#} is selected from the group consisting C₁₋₆ alkyl and C₁₋₆ cycloalkyl; c) an aryl; d) an aryl substituted with at least one from the group consisting of halogen; cyano; hydroxy; carboxyl; carboxamid; carboxamid substituted with at least one from the group consisting of C₁₋₆ alkyl and C₃₋₆ cycloalkyl; C₁₋₆ alkyl; C₁₋₆ alkyl substituted with at least one from the group consisting of halogen, hydroxy, and R^{&}—CONH— wherein R^{&} is selected from the group consisting C₁₋₆ alkyl and C₁₋₆ cycloalkyl; C₁₋₆ cycloalkyl; C₁₋₆ cycloalkyl substituted with at least one from the group consisting of halogen, hydroxy, and R^{&}—CONH— wherein R^{%} is selected from the group consisting C₁₋₆ alkyl and C₁₋₆ cycloalkyl; C₁₋₆ alkoxy; C₁₋₆ alkoxy substituted with at least one from the group consisting of halogen, hydroxy, and R^{§} —CONH— wherein R^{§} is selected from the group consisting C₁₋₆ alkyl and C₁₋₆ cycloalkyl; C₃₋₆cycloalkoxy; C₃₋₆cycloalkoxy substituted with at least one from the group consisting of halogen, hydroxy, and R*—CONH— wherein R* is selected from the group consisting C₁₋₆ alkyl and C₁₋₆ cycloalkyl; amino; amino substituted with at least one from the group consisting of C₁₋₆ alkyl and C₁₋₆ cycloalkyl; and R**—CONH— wherein R** is selected from the group consisting C₁₋₆ alkyl and C₁₋₆ cycloalkyl; e) a C₄₋₁₀ cycloalkyl, a C₄₋₁₀ cycloalkyl substituted with at least one from the group consisting of cyano, halogen, C₁₋₆ alkyl, C₁₋₆ alkyl substituted with at least one from the group consisting of halogen, hydroxy and C₁₋₆ alkyl, C₁₋₆ alkoxy, C₁₋₆ alkoxy substituted with at least one from the group consisting of halogen, hydroxy and C₁₋₆ alkyl, hydroxy, and R^{##}—CONH— wherein R^{##} is selected from the group consisting C₁₋₆ alkyl and C₁₋₆ cycloalkyl; and g) a heterocycle substituted with at least one from the group consisting of halogen; cyano; hydroxy; carboxyl; carboxamid; carboxamid substituted with at least one from the group consisting of C₁₋₆ alkyl and C₃₋₆ cycloalkyl; C₁₋₆ alkyl; C₁₋₆ alkyl substituted with at least one from the group consisting of halogen, hydroxy, and R^{&&}—CONH— wherein R^{&&} is selected from the group consisting C₁₋₆ alkyl and C₁₋₆ cycloalkyl; C₁₋₆ cycloalkyl; C₁₋₆ cycloalkyl substituted with at least one from the group consisting of halogen, hydroxy, and R^{%%}—CONH— wherein R^{%%} is selected from the group consisting C₁₋₆ alkyl and C₁₋₆ cycloalkyl; C₁₋₆ alkoxy; C₁₋₆ alkoxy substituted with at least one from the group consisting of halogen, hydroxy, and R^{§§} —CONH— wherein R^{§§} is selected from the group consisting C₁₋₆ alkyl and C₁₋₆ cycloalkyl; C₃₋₆ cycloalkoxy; C₃₋₆ cycloalkoxy substituted with at least one from the group consisting of halogen, hydroxy, and R^{a}—CONH— wherein R^{a} is selected from the group consisting C₁₋₆ alkyl and C₁₋₆ cycloalkyl; amino; amino substituted with at least one from the group consisting of C₁₋₆ alkyl and C₁₋₆ cycloalkyl; R^{aa}—CONH— wherein R^{aa} is selected from the group consisting C₁₋₆ alkyl and C₁₋₆ cycloalkyl; a heteroaryl substituted with at least one from the group consisting of a halogen; an amino; an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; an aryl; an aryl substituted with at least one from the group consisting of a halogen, cyano, C₁₋₆ alkoxy, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a heteroaryl; a heteroaryl substituted with at least one from the group consisting of halogen, cyano, alkoxy, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₃₋₇ cycloalkyl, C₃₋₇ cycloalkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₃₋₇ cycloalkoxy, and C₃₋₇ cycloalkoxy substituted with at least one from the group consisting of a halogen, an amino, and an amino substituted with at least one from the group consisting of halogen, C₁₋₆ alkoxy, alkyl, nitro, thio, C₁₋₆ alkylthio, amino, hydroxy and C₁₋₆ carbonyl; a C₁₋₆ carbonyl; and a C₁₋₆ carbonyl substituted with at least one from the group consisting of halogen, C₁₋₆ alkoxy, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, and an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; or an in vivo metabolizable group of B¹;

R¹ is selected from the group consisting of hydrogen and an in vivo metabolizable group;

R² is selected from the group consisting of hydrogen and an in vivo metabolizable group;

R³ is selected from the group consisting of hydrogen and an in vivo metabolizable group;

or a pharmaceutically acceptable salt or solvate thereof;

with the proviso that at least one in vivo metabolizable group is present in at least one from the group consisting of A¹, X¹, B¹, R¹, R² and R³.

In one embodiment of the present invention the prodrug compound is bioactivated outside a mammalian cell, such as a human cell.

In another embodiment of the present invention the prodrug compound is bioactivated inside a mammalian cell, such as a human cell.

In a further embodiment of the present invention the in vivo metabolizable group is selected from the group consisting of a carbamate, an ether, a phosphate, a sulphate, an oxy alkyl phosphate, an oxy alkyl sulphate, an N-Mannich base, a carbonate, an amide, an ester, an N-acylsulphoneamide, a sulfonamide, an imine, an acyloxyalkylamine, a phosphoroimidate, an azoconjugate, a carbonyloxymethyl, an acethylthioethanol, a dithioethanol, a cyclosal, a Hep-direct, a phosphorodiimidatesm ProTide phosphoroimidate, a Pro Tide phosphonoimidate, an alkoxyalkylmonoeter and an acetyl.

In a still further embodiment of the present invention the prodrug compound is selected from a compound of formula II

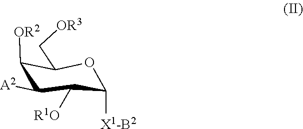

wherein the pyranose ring is α-D-galactopyranose,
wherein A² is selected from

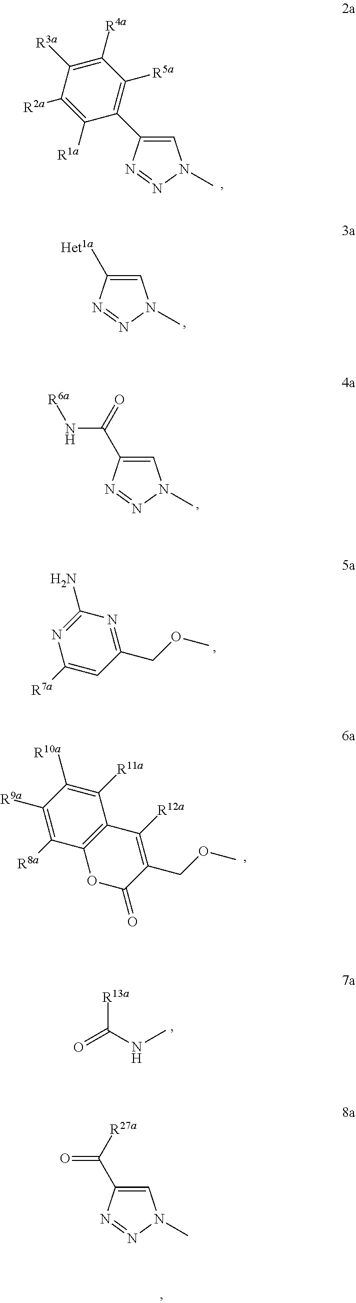

wherein Het^{1a} is selected from a five or six membered heteroaromatic ring, optionally substituted with a group selected from Br; F; Cl; CN; NR^{19a}R^{20a}, wherein R^{19a} and R^{20a} are independently selected from H, C₁₋₃ alkyl, cyclopropyl, iso-propyl, —C(═O)—R^{21a}, wherein R^{21a} is selected from H and C₁₋₃ alkyl; C₁₋₃ alkyl, optionally substituted with a F; cyclopropyl, optionally substituted with a F; iso-propyl, optionally substituted with a F; O-cyclopropyl optionally substituted with a F; O-isopropyl optionally substituted with a F; and OC₁₋₃ alkyl optionally substituted with a F;

wherein R^{1a}-R^{5a} are independently selected from H, CN, NH₂, F, methyl optionally substituted with a F, and OCH₃ optionally substituted with a F;

wherein R^{6a} is selected from C₁₋₆ alkyl optionally substituted with a halogen, branched C₃₋₆ alkyl and C₃₋₇ cycloalkyl;

wherein R^{7a} is selected from a five or six membered heteroaromatic ring, optionally substituted with a group selected from Br, F, Cl, methyl optionally substituted with a F, and OCH₃ optionally substituted with a F, and a phenyl optionally substituted with a group selected from Br, F, Cl, methyl optionally substituted with a F, and OCH₃ optionally substituted with a F;

wherein R^{8a}-R^{12a} are independently selected from H, F, methyl optionally substituted with a F, and OCH₃ optionally substituted with a F;

wherein R^{13a} is a five or six membered heteroaromatic ring optionally substituted with a group selected from H, OH, F, methyl optionally substituted with a F, and OCH₃ optionally substituted with a F, or an aryl, such as phenyl or naphthyl, optionally substituted with a group selected from H, OH, F, methyl optionally substituted with a F, and OCH₃ optionally substituted with a F;

X¹ is selected from S, SO, SO₂, O, C═O, and CR^{32a}R^{33a} wherein R^{32a} and R^{33a} are independently selected from hydrogen, OH, or halogen;

wherein R^{27a} is selected from a C₁₋₆ alkyl, branched C₃₋₆ alkyl, C₁₋₆ alkoxy and branched C₃₋₆ alkoxy;

B² is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{14a}—CONH— wherein R^{14a} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{15a}—CONH— wherein R^{15a} is selected from C₁₋₃ alkyl and cyclopropyl; b) an aryl, such as phenyl or naphthyl, optionally substituted with a group selected from a halogen; CN; —COOH; —CONR^{22a}R^{23a}, wherein R^{22a} and R^{23a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28a}R^{29a}, wherein R^{28a} and R^{29a} are independently selected from H, C₁₋₃ alkyl and isopropyl; OH; and R^{16a}—CONH— wherein R^{16a} is selected from C₁₋₃ alkyl and cyclopropyl; c) a C₅₋₇ cycloalkyl, optionally substituted with a substituent selected from a halogen, CN, methyl optionally substituted with a F, OCH₃ optionally substituted with a F, OCH₂CH₃ optionally substituted with a F, OH, and R^{17a}—CONH— wherein R^{17a} is selected from C₁₋₃ alkyl and cyclopropyl; and d) a heterocycle, such as heteroaryl or heterocycloalkyl, optionally substituted with a group selected from a halogen; CN; —COOH; —CONR^{24a}R^{25a}, wherein R^{24a} and R^{25a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30a}R^{31a}, wherein R^{30a} and R^{31a} are independently selected from H, C₁₋₃ alkyl and isopropyl; OH; and R^{18a}—CONH— wherein R^{18a} is selected from C₁₋₃ alkyl and cyclopropyl; e) a C₁₋₆ alkyl or branched C₃₋₆ alkyl;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³.

In a further embodiment of the present invention the prodrug compound is selected from a compound of formula II

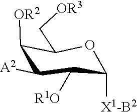

wherein the pyranose ring is α-D-galactopyranose,

### A2 is

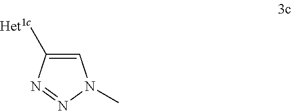

wherein Het^{1c} is a five or six membered heteroaromatic ring selected from the group consisting of formulas 2 to 9:

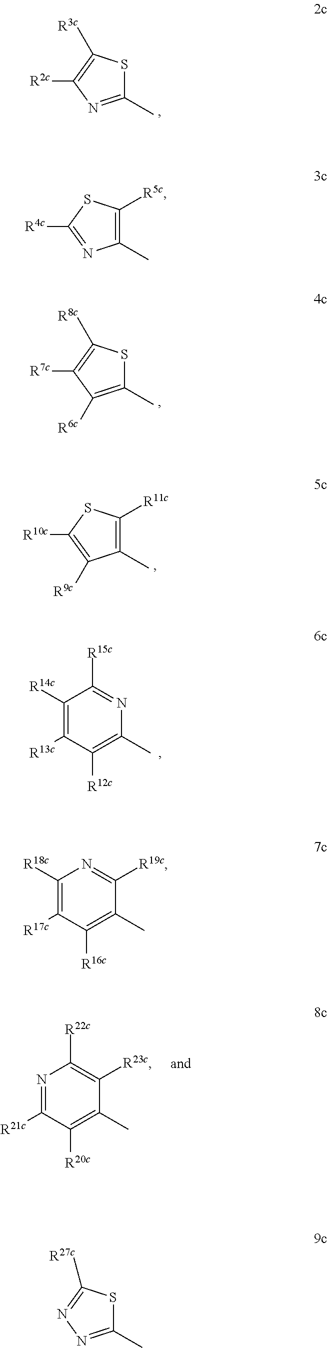

wherein R^{2c} to R^{23c} and R^{27c} are independently selected from H; halogen; OH; CN; SH; S—C₁₋₃ alkyl; C₁₋₃ alkyl, optionally substituted with a F; cyclopropyl, optionally substituted with a F; iso-propyl, optionally substituted with a F; O-cyclopropyl optionally substituted with a F; O-isopropyl optionally substituted with a F; OC₁₋₃ alkyl optionally substituted with a F; NR^{24c}R^{25c}, wherein R^{24c} is selected from H, and C₁₋₃ alkyl, and R^{25c} is selected from H, C₁₋₃ alkyl, and COR^{26c}, wherein R^{26c} is selected from H, and C₁₋₃ alkyl;
X¹ is selected from S, SO, SO₂;
B² is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{27#}—CONH— wherein R^{27#} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{28c}—CONH— wherein R^{28c} is selected from C₁₋₃ alkyl and cyclopropyl; b) an aryl, such as phenyl or naphthyl, optionally substituted with a group selected from a halogen; CN; —COOH; —CONR^{29c}R^{30c}, wherein R^{29c} and R^{39c} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{31c}R^{32c}, wherein R^{31c} and R^{32c} are independently selected from H, C₁₋₃ alkyl and isopropyl; OH; and R^{33c}—CONH—, wherein R^{33c} is selected from C₁₋₃ alkyl and cyclopropyl; c) a C₅₋₇ cycloalkyl, optionally substituted with a substituent selected from a halogen, CN, methyl optionally substituted with a F, OCH₃ optionally substituted with a F, OCH₂CH₃ optionally substituted with a F, OH, and R^{34c}—CONH— wherein R^{34c} is selected from C₁₋₃ alkyl and cyclopropyl; and d) a heterocycle, such as heteroaryl or heterocycloalkyl, optionally substituted with a group selected from a halogen; CN; —COOH; —CONR^{35c}R^{36c}, wherein R^{35c} and R^{36c} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{37c}R^{38c}, wherein R^{37c} and R^{38c} are independently selected from H, C₁₋₃ alkyl and isopropyl; OH; and R^{39c}—CONH— wherein R^{39c} is selected from C₁₋₃ alkyl and cyclopropyl; e) a C₁₋₆ alkyl or branched C₃₋₆ alkyl;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³.

In a still further embodiment of the present invention the prodrug compound is selected from a compound of formula II

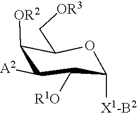

wherein the pyranose ring is α-D-galactopyranose,

### A2 is

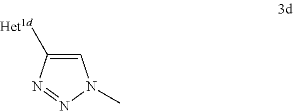

wherein the pyranose ring is α-D-galactopyranose,
Het^{1d} is selected from the group consisting of

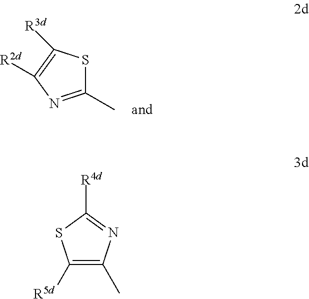

wherein R^{2d} is selected from the group consisting of OH and halogen;

R^{3d} is selected from the group consisting of hydrogen, C₁₋₆ alkyl and halogen;

R^{4d} is selected from the group consisting of OH and halogen;

R^{5d} is selected from the group consisting of hydrogen, C₁₋₆ alkyl and halogen;

X¹ is S;

B² is selected from a) an aryl, such as phenyl or naphthyl, optionally substituted with a group selected from a halogen; CN; —COOH; —CONR^{29d}R^{30d}, wherein R^{29d} and R^{30d} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; SC₁₋₃ alkyl, optionally substituted with a F; O-cyclopropyl, optionally substituted with a F; O-isopropyl, optionally substituted with a F; NR^{31d}R^{32d}, wherein R^{31d} and R^{32d} are independently selected from H, C₁₋₃ alkyl and isopropyl; OH; and R^{33d}—CONH—, wherein R^{33d} is selected from C₁₋₃ alkyl and cyclopropyl; b) a heterocycle, such as heteroaryl or heterocycloalkyl, optionally substituted with a group selected from a halogen; CN; —COOH; —CONR^{35d}R^{36d}, wherein R^{35d} and R^{36d} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; SC₁₋₃ alkyl, optionally substituted with a F; O-isopropyl, optionally substituted with a F; NR^{37d}R^{38d}, wherein R^{37d} and R^{38d} are independently selected from H, C₁₋₃ alkyl and isopropyl; OH; and R^{39d}—CONH— wherein R^{39d} is selected from C₁₋₃ alkyl and cyclopropyl;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³.

In a further embodiment of the present invention the prodrug compound is selected from a compound of formula II

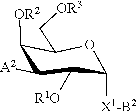

wherein the pyranose ring is α-D-galactopyranose,
A² is selected from

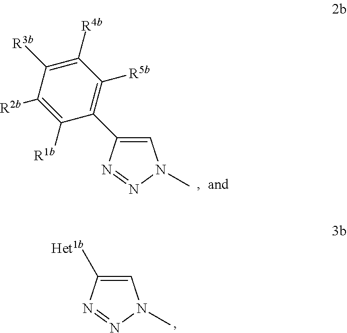

wherein Het^{1b} is selected from a pyridinyl, optionally substituted with a group selected from H, CN, Br, Cl, I, F, methyl optionally substituted with a F, OCH₃ optionally substituted with a F, and SCH₃ optionally substituted with a F; or a pyrimidyl, optionally substituted with a group selected from H, CN, Br, Cl, I, F, methyl optionally substituted with a F, OCH₃ optionally substituted with a F, and SCH₃ optionally substituted with a F;

wherein R^{1b}-R^{5b} are independently selected from a group consisting of H, CN, Br, Cl, I, F, methyl optionally substituted with a F, OCH₃ optionally substituted with a F, and SCH₃ optionally substituted with a F;

X¹ is selected from S, SO, and SO₂;

B² is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{14b}—CONH— wherein R^{14b} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{15b}—CONH— wherein R^{15b} is selected from C₁₋₃ alkyl and cyclopropyl; b) an aryl, such as phenyl or naphthyl, optionally substituted with a group selected from a halogen; CN; —COOH; —CONR^{22b}R^{23b}, wherein R^{22b} and R^{23b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28b}R^{29b}, wherein R^{28b} and R^{29b} are independently selected from H, C₁₋₃ alkyl and isopropyl; OH; and R^{16b}—CONH— wherein R^{16b} is selected from C₁₋₃ alkyl and cyclopropyl; c) a C₅₋₇ cycloalkyl, optionally substituted with a substituent selected from a halogen, CN, methyl optionally substituted with a F, OCH₃ optionally substituted with a F, OCH₂CH₃ optionally substituted with a F, OH, and R^{17b}—CONH— wherein R^{17b} is selected from C₁₋₃ alkyl and cyclopropyl; and d) a heterocycle, such as heteroaryl or heterocycloalkyl, optionally substituted with a group selected from a halogen; CN; —COOH; —CONR^{24b}R^{25b}, wherein R^{24b} and R^{25b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30b}R^{31b}, wherein R^{30b} and R^{31b} are independently selected from H, C₁₋₃ alkyl and isopropyl; OH; and R^{18b}—CONH— wherein R^{18b} is selected from C₁₋₃ alkyl and cyclopropyl; e) a C₁₋₆ alkyl or branched C₃₋₆ alkyl;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³. In a particular embodiment of the above prodrug compound of formula II A² is

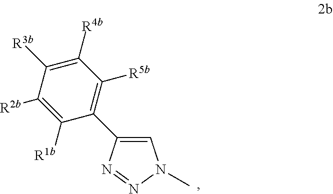

wherein

R^{1b}-R^{5b} are independently selected from a group consisting of H, CN, Br, Cl, I, F, methyl optionally substituted with a F, OCH₃ optionally substituted with a F, and SCH₃ optionally substituted with a F;

X¹ is S;

B² is selected from b) a phenyl, optionally substituted with a group selected from a halogen; CN; —COOH; —CONR^{22b}R^{23b}, wherein R^{22b} and R^{23b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28b}R^{29b}, wherein R^{28b} and R^{29b} are independently selected from H, C₁₋₃ alkyl and isopropyl; OH; and R^{16b}—CONH— wherein R^{16b} is selected from C₁₋₃ alkyl and cyclopropyl; d) a heteroaryl, optionally substituted with a group selected from a halogen; CN; —COOH; —CONR^{24b}R^{25b}, wherein R^{24b} and R^{25b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30b}R^{31b} wherein R^{30b} and R^{31b} are independently selected from H, C₁₋₃ alkyl and isopropyl; OH; and R^{18b}—CONH— wherein R^{18b} is selected from C₁₋₃ alkyl and cyclopropyl;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³. In a more preferred embodiment of the above prodrug compound of formula II A² is

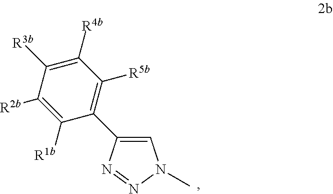

wherein

R^{1b}-R^{5b} are independently selected from a group consisting of H and F;

X¹ is S;

B² is selected from b) a phenyl substituted with a halogen; and d) a heteroaryl substituted with a halogen;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³. In a most preferred embodiment of the above prodrug compound of formula II A² is

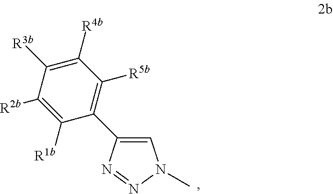

wherein

R^{1b} and R^{5b} are hydrogen, and at least one of R^{2b}-R^{4b} is F;

X¹ is S;

B² is selected from b) a phenyl substituted with a Cl; and d) a pyridinyl substituted with a Br;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³.

In another preferred embodiment of the above prodrug compound of formula II A² is

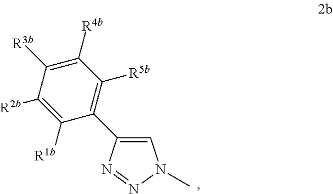

wherein

R^{1b}-R^{5b} are independently selected from a group consisting of H, Cl and F;

X¹ is S;
B² is selected from b) a phenyl substituted with a halogen; and d) a heteroaryl substituted with a cyano, a halogen, or a cyano and a halogen;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³. Preferably, A² is

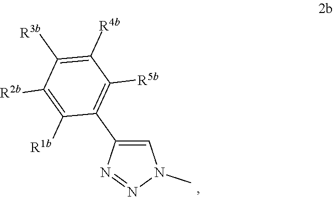

wherein
R^{1b} and R^{5b} are hydrogen, and R^{2b}-R^{4b} is selected from the group consisting of Cl and F;
X¹ is S;
B² is selected from d) a pyridinyl substituted with a group selected from Cl, Br and CN; (typically Cl and Br);
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³. Preferably at least one of R^{2b}-R^{4b} is Cl. Typically R^{2b} and R^{4b} is F and R^{3b} is Cl.

In a further embodiment of the present invention

R¹ is selected from the group consisting of hydrogen and an in vivo metabolizable group;

R² is selected from the group consisting of hydrogen and an in vivo metabolizable group;

R³ is selected from the group consisting of hydrogen and an in vivo metabolizable group;

with the proviso that one in vivo metabolizable group is present in one from the group consisting of R¹, R² and R³.

In a still further embodiment of the present invention R¹ is an in vivo metabolizable group; R² is hydrogen; and R³ is hydrogen.

In a further embodiment of the present invention R¹ is hydrogen; R² is an in vivo metabolizable group; and R³ is hydrogen.

In a still further embodiment of the present invention R¹ is hydrogen; R² is hydrogen; and R³ is an in vivo metabolizable group.

In a further embodiment of the present invention the in vivo metabolizable group is independently selected from the group consisting of carbamate, ether, phosphate, sulphate, oxy alkyl phosphate, oxy alkyl sulphate, N-Mannich base, carbonate, amide, ester, N-acylsulphoneamides, sulfonamides, imines, acyloxyalkylamines, phosphates, phosphoroimidates, azoconjugates, carbonyloxymethyl, acethylthioethanol, dithioethanol, cyclosal, Hep-direct, phosphorodiimidatesm ProTide phosphoroimidates, Pro Tide phosphonoimidates, alkoxyalkylmonoeters and acetyl.

In a still further embodiment of the present invention the compound is selected from the group consisting of
5-Bromopyridin-3-yl 3-deoxy-3-[4-(3,4,5-trifluorophenyl)-1H-1,2,3-triazol-1-yl]-6-O-phospho-1-thio-α-D-galactopyranoside,
5-Bromopyridin-3-yl 3-deoxy-3-[4-(3,4,5-trifluorophenyl)-1H-1,2,3-triazol-1-yl]-2-O-phospho-1-thio-α-D-galactopyranoside,
3,4-Dichlorphenyl 3-deoxy-3-[4-(3,4,5-trifluorophenyl)-1H-1,2,3-triazol-1-yl]-2-O-phospho-1-thio-α-D-galactopyranoside,
3,4-Dichlorphenyl 3-deoxy-3-[4-(3,4,5-trifluorophenyl)-1H-1,2,3-triazol-1-yl]-2-O-sulfo-1-thio-α-D-galactopyranoside,
5-Bromopyridin-3-yl 3-Deoxy-3-[4-(3,4,5-trifluorophenyl)-1H-1,2,3-triazol-1-yl]-2-O-sulfo-1-thio-α-D-galactopyranoside,
5-Bromopyridin-3-yl 3-Deoxy-3-[4-(3,4,5-trifluorophenyl)-1H-1,2,3-triazol-1-yl]-6-O-sulfo-1-thio-α-D-galactopyranoside,
5-Bromopyridin-3-yl 3-deoxy-3-[4-(3,4,5-trifluorophenyl)-1H-1,2,3-triazol-1-yl]-6-O-[(phosphonooxy)methyl]-1-thio-α-D-galactopyranoside,
5-Bromopyridin-3-yl 3-deoxy-3-[4-(3,4,5-trifluorophenyl)-1H-1,2,3-triazol-1-yl]-2-O-[(phosphonooxy)methyl]-1-thio-α-D-galactopyranoside, and
5-Bromopyridin-3-yl 3-[4-(4-chloro-3,5-difluorophenyl)-1H-1,2,3-triazol-1-yl]-3-deoxy-2-O-[(phosphonooxy)methyl]-1-thio-α-D-galactopyranoside; or a
   pharmaceutically acceptable salt or solvate thereof.

In a further aspect the present invention relates to a prodrug compound of the present invention for use as a medicine.

In a still further aspect the present invention relates to a pharmaceutical composition comprising the compound of any one of the previous claims and optionally a pharmaceutically acceptable additive, such as a carrier and/or excipient.

In a further aspect the present invention relates to a prodrug compound of the present invention for use in a method for treating a disorder relating to the binding of a galectin-3 to a ligand in a mammal, such as a human. In a further embodiment the disorder is selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistens; obesity; Diastolic HF; asthma and other interstitial lung diseases, including Hermansky-Pudlak syndrome, mesothelioma; liver disorders, such as non-alcoholic steatohepatitis.

In a still further aspect the present invention relates to a method for treatment of a disorder relating to the binding of a galectin-3 to a ligand in a mammal, such as a human, wherein a therapeutically effective amount of at least one prodrug compound of the present invention is administered to a mammal in need of said treatment. In a further embodiment of the present invention, the disorder is selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistens; obesity; Diastolic (HF) heart failure; asthma and other interstitial lung diseases, including Hermansky-Pudlak syndrome, mesothelioma; liver disorders, such as non-alcoholic steatohepatitis.

Another aspect of the present invention concerns combination therapy involving administering a prodrug compound of the present invention together with a therapeutically active compound different from the prodrug compound of the present invention (interchangeable with “a different therapeutically active compound”). In one embodiment the present invention relates to a combination of a prodrug compound of the present invention and a different therapeutically active compound for use in treatment of a disorder relating to the binding of a galectin-3 to a ligand in a mammal. Such disorders are disclosed below.

In an embodiment of the present invention, a therapeutically effective amount of at least one prodrug compound of the present invention is administered to a mammal in need thereof in combination with a different therapeutically active compound. In a further embodiment, said combination of a prodrug compound together with a different therapeutically active compound is administered to a mammal suffering from a disorder selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistens; obesity; Diastolic HF; asthma and other interstitial lung diseases, including Hermansky-Pudlak syndrome, mesothelioma; liver disorders, such as non-alcoholic steatohepatitis.

A non-limiting group of cancers given as examples of cancers that may be treated, managed and/or prevented by administration of a prodrug compound of the present invention in combination with a different therapeutically active compound is selected from: colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumors, endometrial cancer, gastric cancer, cancer of the head and neck, chronic lymphoid leukemia, hairy cell leukemia and thyroid cancer.

In some aspects of the present invention, the administration of at least one prodrug compound of of the present invention and at least one additional therapeutic agent demonstrates therapeutic synergy. In some aspects of the methods of the present invention, a measurement of response to treatment observed after administering both at least one prodrug compound of the present invention and the additional therapeutic agent is improved over the same measurement of response to treatment observed after administering either the at least one prodrug compound of the present invention or the additional therapeutic agent alone.

A further aspect of the present invention concerns combination therapy involving administering a prodrug compound of the present invention together with an anti-fibrotic compound different from the prodrug compound of the present invention to a mammal in need thereof. In a further embodiment, such anti-fibrotic compound may be selected from the following non-limiting group of anti-fibrotic compounds: pirfenidone, nintedanib, simtuzumab (GS-6624, AB0024), BG00011 (STX100), PRM-151, PRM-167, PEG-FGF21, BMS-986020, FG-3019, MN-001, IW001, SAR156597, GSK2126458, and PBI-4050.

A still further aspect of the present invention concerns combination therapy involving administering a prodrug compound of the present invention in combination with a further conventional cancer treatment such as chemotherapy or radiotherapy, or treatment with immunostimulating substances, gene therapy, treatment with antibodies and treatment using dendritic cells, to a mammal in need thereof.

In an embodiment the prodrug compound of the present invention is administered together with at least one additional therapeutic agent selected from an antineoplastic chemotherapy agent. In a further embodiment, the antineoplastic chemotherapeutic agent is selected from: all-trans retinoic acid, Actimide, Azacitidine, Azathioprine, Bleomycin, Carboplatin, Capecitabine, Cisplatin, Chlorambucil, Cyclophosphamide, Cytarabine, Daunorubicin, Docetaxel, Doxifluridine, Doxorubicin, Epirubicin, Etoposide, Fludarabine, Fluorouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pemetrexed, Revlimid, Temozolomide, Teniposide, Thioguanine, Valrubicin, Vinblastine, Vincristine, Vindesine and Vinorelbine. In one embodiment, a chemotherapeutic agent for use in the combination of the present agent may, itself, be a combination of different chemotherapeutic agents. Suitable combinations include FOLFOX and IFL. FOLFOX is a combination which includes 5-fluorouracil (5-FU), leucovorin, and oxaliplatin. IFL treatment includes irinotecan, 5-FU, and leucovorin.

In a further embodiment of the present invention, the further conventional cancer treatment includes radiation therapy. In some embodiments, radiation therapy includes localized radiation therapy delivered to the tumor. In some embodiments, radiation therapy includes total body irradiation.

In other embodiments of the present invention the further cancer treatment is selected from the group of immunostimulating substances e.g. cytokines and antibodies. Such cytokines may be selected from the group consisting of, but not limited to: GM-CSF, type I IFN, interleukin 21, interleukin 2, interleukin 12 and interleukin 15. The antibody is preferably an immunostimulating antibody such as anti-CD40 or anti-CTLA-4 antibodies. The immunostimulatory substance may also be a substance capable of depletion of immune inhibitory cells (e.g. regulatory T-cells) or factors, said substance may for example be E3 ubiquitin ligases. E3 ubiquitin ligases (the HECT, RING and U-box proteins) have emerged as key molecular regulators of immune cell function, and each may be involved in the regulation of immune responses during infection by targeting specific inhibitory molecules for proteolytic destruction. Several HECT and RING E3 proteins have now also been linked to the induction and maintenance of immune self-tolerance: c-Cbl, Cbl-b, GRAIL, Itch and Nedd4 each negatively regulate T cell growth factor production and proliferation.

In some embodiments of the present invention the prodrug compound of the present invention is administered together with at least one additional therapeutic agent selected from a checkpoint inhibitor. In some embodiments of the invention, the checkpoint inhibitor is acting on one or more of the following, non-limiting group of targets: CEACAM1, galectin-9, TIM3, CD80, CTLA4, PD-1, PD-L1, HVEM, BTLA, CD160, VISTA, B7-H4, B7-2, CD155, CD226, TIGIT, CD96, LAG3, GITF, OX40, CD137, CD40, IDO, and TDO. These are known targets and some of these targets are described in Melero et al., Nature Reviews Cancer (2015).

In some embodiments of the present invention the prodrug compound of the present invention is administered together with at least one additional therapeutic agent selected from an inhibitor of indoleamine-2,3-dioxygenase (IDO).

In some embodiments of the present invention the prodrug compound of the present invention is administered together with at least one additional therapeutic agent selected from one or more inhibitors of the CTLA4 pathway. In some embodiments, the inhibitor of the CTLA4 pathway is selected from one or more antibodies against CTLA4.

In some embodiments of the present invention the prodrug compound of the present invention is administered together with at least one additional therapeutic agent selected from one or more inhibitors of the PD-1/PD-L pathway. In some embodiments, the one or more inhibitors of the PD-1/PD-L pathway are selected from one or more antibodies against PD-1, PD-L1, and/or PD-L2.

In a still further aspect the present invention relates to a process of preparing a compound of formula I and II wherein A¹, A², X¹, B¹ and B² are defined as above under formula I and II, R¹ and R² are defined as hydrogen and R³ is an in vivo metabolizable group, such as a sulfate, phosphate or methylphosphate or a pharmaceutically acceptable salt thereof comprising the step a1);
a1) Reacting a compound of Formula I or II, wherein R¹ and R² is either hydrogen or protected with a protection group such as benzoyl, with a reagent such as POCl₃ in the presence of a base such as DIEA to give an intermediate which is treated with water to give a compound of formula I or II wherein R³ is phosphate ester (—OP(═O)OH₂); or with a reagent such as sulfurtrioxide-triethylamine complex to give R³ is sulfate (—SO₂OH); or with a reagent such as paraformaldehyde followed by tetrabutylammouniumphosphate to give a compound of formula I or II wherein R³ is methylphosphate (CH₂OP(═O)OH₂); or with a reagent such as di-tertbutyl chloromethylphosphate in the presence of AgO and NaI to give an intermediate which is treated with TFA to give a compound of formula I or II wherein R³ is methylphosphate (CH₂OP(═O)OH₂)

In a still further aspect the present invention relates to a process of preparing a compound of formula I and II wherein A¹, A², X¹, B¹ and B² are defined as above under formula I and II, R² and R³ are defined as hydrogen and R¹ is an in vivo metabolizable group, such as a sulfate, phosphate or methylphosphate or a pharmaceutically acceptable salt thereof comprising the step a2);
a2) Reacting a compound of Formula I or II, wherein R² and R³ is protected with a protection group such as benzylidene, with a reagent such as POCl₃ in the presence of a base such as DIEA to give an intermediate which is treated with water to give a compound of formula I or II wherein R¹ is phosphate ester (—OP(═O)OH₂); or with a reagent such as sulfurtrioxide-triethylamine complex to give a compound of formula I or II wherein R¹ is sulfate (—SO₂OH); or with a reagent such as paraformaldehyde followed by tetrabutylammouniumphosphate to give a compound of formula I or II wherein R¹ is methylphosphate (CH₂OP(═O)OH₂); or with a reagent such as di-tertbutyl chloromethylphosphate in the presence of AgO and NaI to give an intermediate which is treated with TFA to give a compound of formula I or II wherein R¹ is methylphosphate (CH₂OP(═O)OH₂)

In a still further aspect the present invention relates to a process of preparing a compound of formula I and II wherein A¹, A², X¹, B¹ and B² are defined as above under formula I and II, R¹ and R³ are defined as hydrogen and R² is an in vivo metabolizable group, such as a sulfate, phosphate or methylphosphate or a pharmaceutically acceptable salt thereof comprising the step a3);
a3) Reacting a compound of Formula I or II, wherein R¹ and R³ is protected with a protection group, with a reagent such as POCl₃ in the presence of a base such as DIEA to give an intermediate which is treated with water to give a compound of formula I or II wherein R² is phosphate ester (—OP(═O)OH₂); or with a reagent such as sulfurtrioxide-triethylamine complex to give a compound of formula I or II wherein R² is sulfate (—SO₂OH); or with a reagent such as paraformaldehyde followed by tetrabutylammouniumphosphate to give a compound of formula I or II wherein R² is methylphosphate (CH₂OP(═O)OH₂); or with a reagent such as di-tertbutyl chloromethylphosphate in the presence of AgO and NaI to give an intermediate which is treated with TFA to give a compound of formula I or II wherein R² is methylphosphate (CH₂OP(═O)OH₂)

### DETAILED DESCRIPTION OF THE INVENTION

The compounds of the present invention are novel prodrugs of galactopyranose compounds that unexpectedly have good solubility and can be used to increase the maximum dose resulting in dose correlated bioavailability. The prodrug compounds of the present invention of formula (I) differ from prior art compounds in that it comprises at least one in vivo hydrolysable group which will be cleaved off upon administration to a mammal, such as a human subject, to create an active metabolite being an inhibitor of galectin 3.

The prodrugs of the present invention convert into an active metabolite which has high affinity to galectin 3 and inhibits galectin 3. Here prodrugs have been developed in which one to three functional group(s) are introduced at selected positions to prepare a prodrug which is charged in the intestine due to it's pKa.

Preferably, the pyranose ring is α-D-galactopyranose which compounds have very good solubility and suitability as prodrugs. In particular, prodrugs having an in vivo hydrolysable group on the C2, C4 and/or C6 of the galactopyranose ring have aqueous solubility above 8 mg/ml, and in some instances above 10 mg/ml.

In broad aspect the present invention concerns a prodrug compound of formula (I)

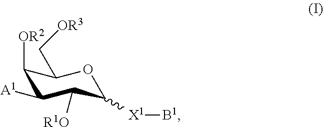

wherein the pyranose ring is α- or β-D-galactopyranose (as indicated by wavy line);
and wherein R¹, R², R³, A¹, X¹ and B¹ are as defined above.

In one embodiment A¹ is selected from a heteroaryl substituted with at least one from the group consisting of a halogen; CN; C₂₋₆ alkenyl; C₂₋₆ alkynyl; carboxyl; C₁₋₆ alkoxy; C₁₋₆ thioalkyl; an amino; an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; an aryl; an aryl substituted with at least one from the group consisting of a halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a heteroaryl; a heteroaryl substituted with at least one from the group consisting of halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₃₋₇ cycloalkyl, C₃₋₇ cycloalkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₃₋₇ cycloalkoxy, and C₃₋₇ cycloalkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a C₁₋₆ carbonyl; a C₁₋₆ carbonyl substituted with at least one from the group consisting of halogen, C₁₋₆ alkoxy, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, and an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl. In a more specific embodiment A¹ is a heteroaryl substituted with an aryl substituted with at least one from the group consisting of a halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl. In a still more specific embodiment A¹ is a triazolyl, such as a 1,2,3-triazolyl, substituted with a phenyl substituted with at least one halogen, such a 1, 2 or 3 F.

In another embodiment X¹ is selected from the group consisting of 0 and S. Preferably X¹ is S.

In a further embodiment B¹ is selected from the group consisting of d) an aryl substituted with at least one from the group consisting of halogen; cyano; hydroxy; carboxyl; carboxamid; carboxamid substituted with at least one from the group consisting of C₁₋₆ alkyl and C₃₋₆ cycloalkyl; C₁₋₆ alkyl; C₁₋₆ alkyl substituted with at least one from the group consisting of halogen, hydroxy, and R^{&-}CONH— wherein R^{&} is selected from the group consisting C₁₋₆ alkyl and C₁₋₆ cycloalkyl; C₁₋₆ cycloalkyl; C₁₋₆ cycloalkyl substituted with at least one from the group consisting of halogen, hydroxy, and R^{%-}CONH— wherein R^{%} is selected from the group consisting C₁₋₆ alkyl and C₁₋₆ cycloalkyl; C₁₋₆ alkoxy; C₁₋₆ alkoxy substituted with at least one from the group consisting of halogen, hydroxy, and R^{§} —CONH— wherein R^{§} is selected from the group consisting C₁₋₆ alkyl and C₁₋₆ cycloalkyl; C₃₋₆ cycloalkoxy; C₃₋₆ cycloalkoxy substituted with at least one from the group consisting of halogen, hydroxy, and R*—CONH— wherein R* is selected from the group consisting C₁₋₆ alkyl and C₁₋₆ cycloalkyl; amino; amino substituted with at least one from the group consisting of C₁₋₆ alkyl and C₁₋₆ cycloalkyl; and R**—CONH— wherein R** is selected from the group consisting C₁₋₆ alkyl and C₁₋₆ cycloalkyl; and g) a heteroaryl substituted with at least one from the group consisting of halogen; cyano; hydroxy; carboxyl; carboxamid; carboxamid substituted with at least one from the group consisting of C₁₋₆ alkyl and C₃₋₆ cycloalkyl; C₁₋₆ alkyl; C₁₋₆ alkyl substituted with at least one from the group consisting of halogen, hydroxy, and R^{&&}—CONH— wherein R^{&&} is selected from the group consisting C₁₋₆ alkyl and C₁₋₆ cycloalkyl; C₁₋₆ cycloalkyl; C₁₋₆ cycloalkyl substituted with at least one from the group consisting of halogen, hydroxy, and R^{%%}—CONH— wherein R^{%%} is selected from the group consisting C₁₋₆ alkyl and C₁₋₆ cycloalkyl; C₁₋₆ alkoxy; C₁₋₆ alkoxy substituted with at least one from the group consisting of halogen, hydroxy, and R^{§§} —CONH— wherein R^{§§} is selected from the group consisting C₁₋₆ alkyl and C₁₋₆ cycloalkyl; C₃₋₆ cycloalkoxy; C₃₋₆ cycloalkoxy substituted with at least one from the group consisting of halogen, hydroxy, and R^{a}—CONH— wherein R^{a} is selected from the group consisting C₁₋₆ alkyl and C₁₋₆ cycloalkyl; amino; amino substituted with at least one from the group consisting of C₁₋₆ alkyl and C₁₋₆ cycloalkyl; R^{aa}—CONH— wherein R^{aa} is selected from the group consisting C₁₋₆ alkyl and C₁₋₆ cycloalkyl; a heteroaryl substituted with at least one from the group consisting of a halogen; an amino; an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; an aryl; an aryl substituted with at least one from the group consisting of a halogen, cyano, C₁₋₆ alkoxy, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a heteroaryl; a heteroaryl substituted with at least one from the group consisting of halogen, cyano, C₁₋₆ alkoxy, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₃₋₇ cycloalkyl, C₃₋₇ cycloalkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₃₋₇ cycloalkoxy, and C₃₋₇ cycloalkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a C₁₋₆ carbonyl; and a C₁₋₆ carbonyl substituted with at least one from the group consisting of halogen, C₁₋₆ alkoxy, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, and an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl. Preferably B¹ is selected from the group consisting of d) a phenyl substituted with at least one from the group consisting of halogen; cyano; hydroxy; carboxyl; carboxamid; carboxamid substituted with at least one from the group consisting of C₁₋₆ alkyl and C₃₋₆ cycloalkyl; C₁₋₆ alkyl; C₁₋₆ alkyl substituted with at least one from the group consisting of halogen, hydroxy, and R^{&-}CONH— wherein R^{&} is selected from the group consisting C₁₋₆ alkyl and C₁₋₆ cycloalkyl; C₁₋₆ cycloalkyl; C₁₋₆ cycloalkyl substituted with at least one from the group consisting of halogen, hydroxy, and R^{%}—CONH— wherein R^{%} is selected from the group consisting C₁₋₆ alkyl and C₁₋₆ cycloalkyl; C₁₋₆ alkoxy; C₁₋₆ alkoxy substituted with at least one from the group consisting of halogen, hydroxy, and R^{§} —CONH— wherein R^{§§} is selected from the group consisting C₁₋₆ alkyl and C₁₋₆ cycloalkyl; C₃₋₆ cycloalkoxy; C₃₋₆ cycloalkoxy substituted with at least one from the group consisting of halogen, hydroxy, and R*—CONH— wherein R* is selected from the group consisting C₁₋₆ alkyl and C₁₋₆ cycloalkyl; amino; amino substituted with at least one from the group consisting of C₁₋₆ alkyl and C₁₋₆ cycloalkyl; and R**—CONH— wherein R** is selected from the group consisting C₁₋₆ alkyl and C₁₋₆ cycloalkyl; and g) a pyridinyl substituted with at least one from the group consisting of halogen; cyano; hydroxy; carboxyl; carboxamid; carboxamid substituted with at least one from the group consisting of C₁₋₆ alkyl and C₃₋₆ cycloalkyl; C₁₋₆ alkyl; C₁₋₆ alkyl substituted with at least one from the group consisting of halogen, hydroxy, and R^{&&}—CONH— wherein R^{&&} is selected from the group consisting C₁₋₆ alkyl and C₁₋₆ cycloalkyl; C₁₋₆ cycloalkyl; C₁₋₆ cycloalkyl substituted with at least one from the group consisting of halogen, hydroxy, and R^{%%}—CONH— wherein R^{%%} is selected from the group consisting C₁₋₆ alkyl and C₁₋₆ cycloalkyl; C₁₋₆ alkoxy; C₁₋₆ alkoxy substituted with at least one from the group consisting of halogen, hydroxy, and R^{§§} —CONH— wherein R^{§§} is selected from the group consisting C₁₋₆ alkyl and C₁₋₆ cycloalkyl; C₃₋₆ cycloalkoxy; C₃₋₆ cycloalkoxy substituted with at least one from the group consisting of halogen, hydroxy, and R^{a}—CONH— wherein R^{a} is selected from the group consisting C₁₋₆ alkyl and C₁₋₆ cycloalkyl; amino; amino substituted with at least one from the group consisting of C₁₋₆ alkyl and C₁₋₆ cycloalkyl; R^{aa}—CONH— wherein R^{aa} is selected from the group consisting C₁₋₆ alkyl and C₁₋₆ cycloalkyl; a heteroaryl substituted with at least one from the group consisting of a halogen; an amino; an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; an aryl; an aryl substituted with at least one from the group consisting of a halogen, cyano, C₁₋₆ alkoxy, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a heteroaryl; a heteroaryl substituted with at least one from the group consisting of halogen, cyano, C₁₋₆ alkoxy, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₃₋₇ cycloalkyl, C₃₋₇ cycloalkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₃₋₇ cycloalkoxy, and C₃₋₇ cycloalkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a C₁₋₆ carbonyl; and a C₁₋₆ carbonyl substituted with at least one from the group consisting of halogen, C₁₋₆ alkoxy, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, and an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl. Most preferably B¹ is selected from the group consisting of d) a phenyl substituted with at least one halogen, such as 1 or 2 Cl; and g) a pyridinyl substituted with at least one halogen, such as 1 Br.

In a further embodiment R¹ is selected from the group consisting of hydrogen and an in vivo metabolizable group;

R² is selected from the group consisting of hydrogen and an in vivo metabolizable group;

R³ is selected from the group consisting of hydrogen and an in vivo metabolizable group;

with the proviso that one in vivo metabolizable group is present in one from the group consisting of R¹, R² and R³. Typically, R¹ is an in vivo metabolizable group; R² is hydrogen; and R³ is hydrogen. Typically, R¹ is hydrogen; R² is hydrogen; and R³ is an in vivo metabolizable group.

In a still further embodiment of the present invention the prodrug compound of formula I is bioactivated outside a mammalian cell, such as a human cell.

In another embodiment of the present invention the prodrug compound of formula I is bioactivated inside a mammalian cell, such as a human cell.

In a further embodiment of the present invention the in vivo metabolizable group is selected from the group consisting of a carbamate, an ether, a phosphate, a sulphate, an oxy alkyl phosphate, an oxy alkyl sulphate, an N-Mannich base, a carbonate, an amide, an ester, an N-acylsulphoneamide, a sulfonamide, an imine, an acyloxyalkylamine, a phosphoroimidate, an azoconjugate, a carbonyloxymethyl, an acethylthioethanol, a dithioethanol, a cyclosal, a Hep-direct, a phosphorodiimidatesm ProTide phosphoroimidate, a Pro Tide phosphonoimidate, an alkoxyalkylmonoeter and an acetyl. Typically, the in vivo metabolizable group is selected from the group consisting of a phosphate, a sulphate, an oxy C₁₋₆ alkyl phosphate, and an oxy C₁₋₆ alkyl sulphate.

In a still further embodiment of the present invention the prodrug compound is selected from a compound of formula II

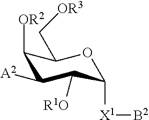

wherein the pyranose ring is α-D-galactopyranose,
wherein

A² is selected from A¹ as defined above;

X¹ is selected from S, SO, SO₂, O, C═O, and CR^{32a}R^{33a} wherein R^{32a} and R^{33a} are independently selected from hydrogen, OH, or halogen;

B² is selected from B¹ as defined above;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³.

In a further embodiment A² is selected from

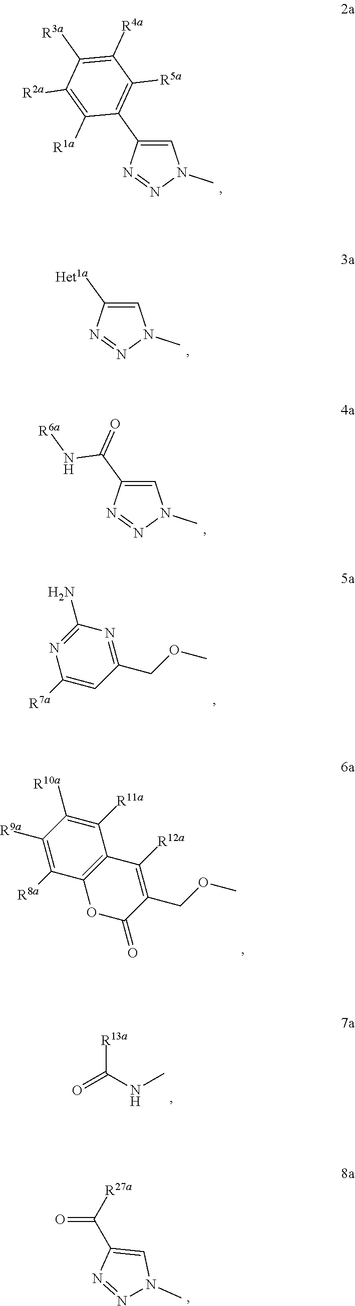

wherein Het^{1a} is selected from a five or six membered heteroaromatic ring, optionally substituted with a group selected from Br; F; Cl; CN; NR^{19a}R^{20a}, wherein R^{19a} and R^{20a} are independently selected from H, C₁₋₃ alkyl, cyclopropyl, iso-propyl, —C(═O)—R^{21a}, wherein R^{21a} is selected from H and C₁₋₃ alkyl; C₁₋₃ alkyl, optionally substituted with a F; cyclopropyl, optionally substituted with a F; iso-propyl, optionally substituted with a F; O-cyclopropyl optionally substituted with a F; O-isopropyl optionally substituted with a F; and OC₁₋₃ alkyl optionally substituted with a F;

wherein R^{1a}-R^{5a} are independently selected from H, CN, NH₂, F, methyl optionally substituted with a F, and OCH₃ optionally substituted with a F;

wherein R^{6a} is selected from C₁₋₆ alkyl optionally substituted with a halogen, branched C₃₋₆ alkyl and C₃₋₇ cycloalkyl;

wherein R^{7a} is selected from a five or six membered heteroaromatic ring, optionally substituted with a group selected from Br, F, Cl, methyl optionally substituted with a F, and OCH₃ optionally substituted with a F, and a phenyl optionally substituted with a group selected from Br, F, Cl, methyl optionally substituted with a F, and OCH₃ optionally substituted with a F;

wherein R^{8a}-R^{12a} are independently selected from H, F, methyl optionally substituted with a F, and OCH₃ optionally substituted with a F;

wherein R^{13a} is a five or six membered heteroaromatic ring optionally substituted with a group selected from H, OH, F, methyl optionally substituted with a F, and OCH₃ optionally substituted with a F, or an aryl, such as phenyl or naphthyl, optionally substituted with a group selected from H, OH, F, methyl optionally substituted with a F, and OCH₃ optionally substituted with a F. Preferably, A² is formula 2a, wherein R^{1a}-R^{5a} are independently selected from H, CN, NH₂, F, methyl optionally substituted with a F, and OCH₃ optionally substituted with a F. More preferred A² is formula 2a, wherein R^{1a} and R^{5a} are hydrogen, and R^{2a}-R^{4a} are independently selected from H, CN, NH₂, F, methyl optionally substituted with a F, and OCH₃ optionally substituted with a F. Most preferred A² is formula 2a, wherein R^{1a} and R^{5a} are hydrogen, and R^{2a}-R^{4a} are independently selected from hydrogen and F. Typically, A² is formula 2a, wherein R^{1a} and R^{5a} are hydrogen, and R^{2a}-R^{4a} are all F.

In a still further embodiment X¹ is selected from S and O. Preferably, X¹ is S.

In a further embodiment B² is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{14a}—CONH— wherein R^{14a} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{15a}—CONH— wherein R^{15a} is selected from C₁₋₃ alkyl and cyclopropyl; b) an aryl, such as phenyl or naphthyl, optionally substituted with a group selected from a halogen; CN; —COOH; —CONR^{22a}R^{23a}, wherein R^{22a} and R^{23a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28a}R^{29a}, wherein R^{28a} and R^{29a} are independently selected from H, C₁₋₃ alkyl and isopropyl; OH; and R^{16a}—CONH— wherein R^{16a} is selected from C₁₋₃ alkyl and cyclopropyl; c) a C₅₋₇ cycloalkyl, optionally substituted with a substituent selected from a halogen, CN, methyl optionally substituted with a F, OCH₃ optionally substituted with a F, OCH₂CH₃ optionally substituted with a F, OH, and R^{17a}—CONH— wherein R^{17a} is selected from C₁₋₃ alkyl and cyclopropyl; and d) a heterocycle, such as heteroaryl or heterocycloalkyl, optionally substituted with a group selected from a halogen; CN; —COOH; —CONR^{24a}R^{25a}, wherein R^{24a} and R^{25a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30a}R^{31a}, wherein R^{30a} and R^{31a} are independently selected from H, C₁₋₃ alkyl and isopropyl; OH; and R^{18a}—CONH— wherein R^{18a} is selected from C₁₋₃ alkyl and cyclopropyl; e) a C₁₋₆ alkyl or branched C₃₋₆ alkyl.

In a still further embodiment B² is selected from b) an aryl, such as phenyl or naphthyl, optionally substituted with a group selected from a halogen; CN; —COOH; —CONR^{22a}R^{23a}, wherein R^{22a} and R^{23a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28a}R^{29a}, wherein R^{28a} and R^{29a} are independently selected from H, C₁₋₃ alkyl and isopropyl; OH; and R^{16a}—CONH— wherein R^{16a} is selected from C₁₋₃ alkyl and cyclopropyl; and d) a heterocycle, such as heteroaryl or heterocycloalkyl, optionally substituted with a group selected from a halogen; CN; —COOH; —CONR^{24a}R^{25a}, wherein R^{24a} and R^{25a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30a}R^{31a}, wherein R^{30a} and R^{31a} are independently selected from H, C₁₋₃ alkyl and isopropyl; OH; and R^{18a}—CONH— wherein R^{18a} is selected from C₁₋₃ alkyl and cyclopropyl. In a further embodiment B² is selected from b) a phenyl optionally substituted with a group selected from a halogen; CN; —COOH; —CONR^{22a}R^{23a}, wherein R^{22a} and R^{23a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28a}R^{29a}, wherein R^{28a} and R^{29a} are independently selected from H, C₁₋₃ alkyl and isopropyl; OH; and R^{16a}—CONH— wherein R^{16a} is selected from C₁₋₃ alkyl and cyclopropyl; and d) a heteroaryl optionally substituted with a group selected from a halogen; CN; —COOH; —CONR^{24a}R^{25a}, wherein R^{24a} and R^{25a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30a}R^{31a}, wherein R^{30a} and R^{31a} are independently selected from H, C₁₋₃ alkyl and isopropyl; OH; and R^{18a}—CONH— wherein R^{18a} is selected from C₁₋₃ alkyl and cyclopropyl. In a still further embodiment B² is selected from b) a phenyl substituted with a halogen; and d) a heteroaryl, such as a pyridinyl, substituted with a halogen.

In a further embodiment R¹ is selected from the group consisting of hydrogen and an in vivo metabolizable group;

R² is selected from the group consisting of hydrogen and an in vivo metabolizable group;

R³ is selected from the group consisting of hydrogen and an in vivo metabolizable group;

with the proviso that one in vivo metabolizable group is present in one from the group consisting of R¹, R² and R³. Typically, R¹ is an in vivo metabolizable group; R² is hydrogen; and R³ is hydrogen. Typically, R¹ is hydrogen; R² is hydrogen; and R³ is an in vivo metabolizable group.

In a further embodiment of the present invention the prodrug compound is selected from a compound of formula II

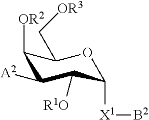

wherein the pyranose ring is α-D-galactopyranose,

A2 is

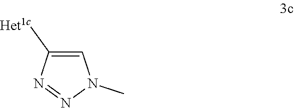

wherein Het^{1c} is a five or six membered heteroaromatic ring selected from the group consisting of formulas 2c to 9c:

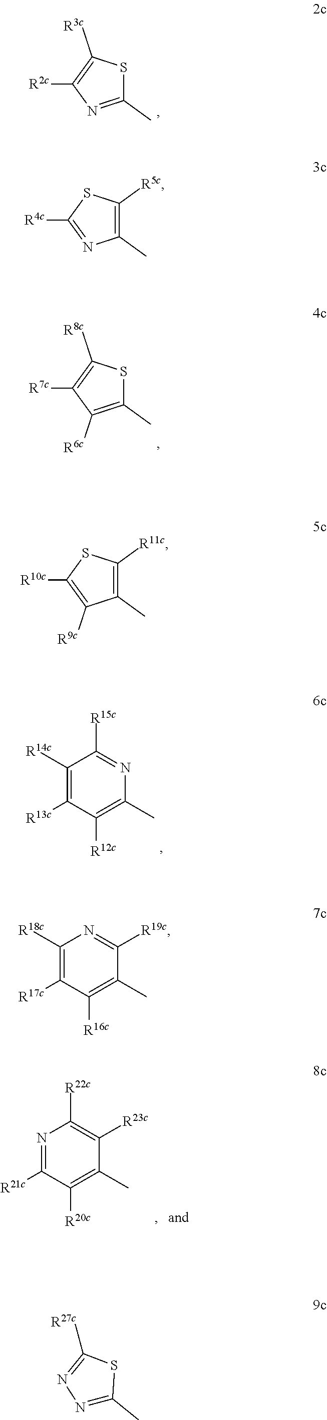

wherein R^{2c} to R^{23c} and R^{27c} are independently selected from H; halogen; OH; CN; SH; S—C₁₋₃ alkyl; C₁₋₃ alkyl, optionally substituted with a F; cyclopropyl, optionally substituted with a F; iso-propyl, optionally substituted with a F; O-cyclopropyl optionally substituted with a F; O-isopropyl optionally substituted with a F; OC₁₋₃ alkyl optionally substituted with a F; NR^{24c}R^{25c}, wherein R^{24c} is selected from H, and C₁₋₃ alkyl, and R^{25c} is selected from H, C₁₋₃ alkyl, and COR^{26c}, wherein R^{26c} is selected from H, and C₁₋₃ alkyl;

X¹ is selected from S;

B² is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{27#}—CONH— wherein R^{27#} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{28c}—CONH— wherein R^{28c} is selected from C₁₋₃ alkyl and cyclopropyl; b) a phenyl optionally substituted with a group selected from a halogen; CN; —COOH; —CONR^{29c}R^{30c}, wherein R^{29c} and R^{39c} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{31c}R^{32c}, wherein R^{31c} and R^{32c} are independently selected from H, C₁₋₃ alkyl and isopropyl; OH; and R^{33c}—CONH—, wherein R^{33c} is selected from C₁₋₃ alkyl and cyclopropyl; c) a C₅₋₇ cycloalkyl, optionally substituted with a substituent selected from a halogen, CN, methyl optionally substituted with a F, OCH₃ optionally substituted with a F, OCH₂CH₃ optionally substituted with a F, OH, and R^{34c}—CONH— wherein R^{34c} is selected from C₁₋₃ alkyl and cyclopropyl; and d) a heterocycle, such as heteroaryl or heterocycloalkyl, optionally substituted with a group selected from a halogen; CN; —COOH; —CONR^{35c}R^{36c}, wherein R^{35c} and R^{36c} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{37c}R^{38c}, wherein R^{37c} and R^{38c} are independently selected from H, C₁₋₃ alkyl and isopropyl; OH; and R^{39c}—CONH— wherein R^{39c} is selected from C₁₋₃ alkyl and cyclopropyl; e) a C₁₋₆ alkyl or branched C₃₋₆ alkyl;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³.

In a still further embodiment of the present invention the prodrug compound is selected from a compound of formula II

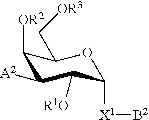

wherein the pyranose ring is α-D-galactopyranose,

A2 is

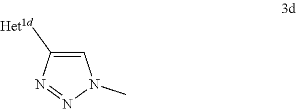

wherein Het^{1d} is selected from the group consisting of

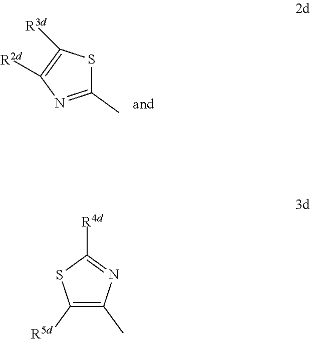

wherein R^{2d} is selected from the group consisting of OH and halogen;

R^{3d} is selected from the group consisting of hydrogen, C₁₋₆ alkyl and halogen;

R^{4d} is selected from the group consisting of OH and halogen;

R^{5d} is selected from the group consisting of hydrogen, C₁₋₆ alkyl and halogen;

X¹ is S;

B² is selected from a) an aryl, such as phenyl or naphthyl, optionally substituted with a group selected from a halogen; CN; —COOH; —CONR^{29d}R^{30d}, wherein R^{29d} and R^{30d} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; SC₁₋₃ alkyl, optionally substituted with a F; O-cyclopropyl, optionally substituted with a F; O-isopropyl, optionally substituted with a F; NR^{31d}R^{32d}, wherein R^{31d} and R^{32d} are independently selected from H, C₁₋₃ alkyl and isopropyl; OH; and R^{33d}—CONH—, wherein R^{33d} is selected from C₁₋₃ alkyl and cyclopropyl; b) a heterocycle, such as heteroaryl or heterocycloalkyl, optionally substituted with a group selected from a halogen; CN; —COOH; —CONR^{35d}R^{36d}, wherein R^{35d} and R^{36d} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; SC₁₋₃ alkyl, optionally substituted with a F; O-isopropyl, optionally substituted with a F; NR^{37d}R^{38d}, wherein R^{37d} and R^{38d} are independently selected from H, C₁₋₃ alkyl and isopropyl; OH; and R^{39d}—CONH— wherein R^{39d} is selected from C₁₋₃ alkyl and cyclopropyl;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³.

In a further embodiment of the present invention the prodrug compound is selected from a compound of formula II

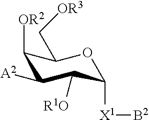

wherein the pyranose ring is α-D-galactopyranose,
wherein
A² is selected from A¹ as defined above;
X¹ is selected from S, SO, SO₂, and O;
B² is selected from B¹ as defined above;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
   with the proviso that at least one in vivo metabolizable group is present in at least one from the group consisting of R¹, R² and R³.

In an embodiment A² is selected from

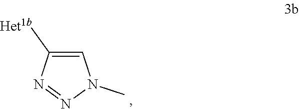

wherein Het^{1b} is selected from a pyridinyl, optionally substituted with a group selected from H, CN, Br, Cl, I, F, methyl optionally substituted with a F, OCH₃ optionally substituted with a F, and SCH₃ optionally substituted with a F; or a pyrimidyl, optionally substituted with a group selected from H, CN, Br, Cl, I, F, methyl optionally substituted with a F, OCH₃ optionally substituted with a F, and SCH₃ optionally substituted with a F.

In another embodiment A² is selected from

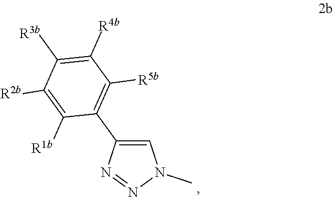

wherein R^{1b}-R^{5b} are independently selected from a group consisting of H, CN, Br, Cl, I, F, methyl optionally substituted with a F, OCH₃ optionally substituted with a F, and SCH₃ optionally substituted with a F. Preferably, A² is formula 2b, and R^{1b} and R^{5b} are both hydrogen, and R^{2b}-R^{4b} are independently selected from a group consisting of H, CN, Br, Cl, I, F, methyl optionally substituted with a F, OCH₃ optionally substituted with a F, and SCH₃ optionally substituted with a F. Typically, A² is formula 2b, and R^{1b} and R^{5b} are both hydrogen, and R^{2b}-R^{4b} are independently selected from a group consisting of H, Br, Cl, I, and F, for instance R^{2b}-R^{4b} are all F, or R^{2b} is F, R^{3b} is Cl and R^{4b} is F.

In a further embodiment X¹ is S.

In a still further embodiment B² is selected from b) a phenyl optionally substituted with a group selected from a halogen; CN; —COOH; —CONR^{22b}R^{23b}, wherein R^{22b} and R^{23b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28b}R^{29b}, wherein R^{28b} and R^{29b} are independently selected from H, C₁₋₃ alkyl and isopropyl; OH; and R^{16b}—CONH— wherein R^{16b} is selected from C₁₋₃ alkyl and cyclopropyl; and d) a heteroaryl optionally substituted with a group selected from a halogen; CN; —COOH; —CONR^{24b}R^{25b}, wherein R^{24b} and R^{25b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30b}R^{31b}, wherein R^{30b} and R^{31b} are independently selected from H, C₁₋₃ alkyl and isopropyl; OH; and R^{18b}—CONH— wherein R^{18b} is selected from C₁₋₃ alkyl and cyclopropyl.

In one embodiment B² is selected from a phenyl optionally substituted with a group selected from a halogen; CN; —COOH; —CONR^{22b}R^{23b}, wherein R^{22b} and R^{23b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28b}R^{29b}, wherein R^{28b} and R^{29b} are independently selected from H, C₁₋₃ alkyl and isopropyl; OH; and R^{16b}—CONH— wherein R^{16b} is selected from C₁₋₃ alkyl and cyclopropyl. Typically, B² is selected from a phenyl substituted with a halogen, such as 1-3 selected from Cl, F, Br, and I. In a particular embodiment B² is selected from a phenyl substituted with 1-3 Cl, such as two Cl. In another particular embodiment B² is selected from a phenyl substituted with one halogen and optionally one or two group(s) selected from CN; —COOH; —CONR^{22b}R^{23b}, wherein R^{22b} and R^{23b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28b}R^{29b}, wherein R^{28b} and R^{29b} are independently selected from H, C₁₋₃ alkyl and isopropyl; OH; and R^{16b}—CONH— wherein R^{1b} is selected from C₁₋₃ alkyl and cyclopropyl.

In another embodiment B² is selected from a heteroaryl optionally substituted with a group selected from a halogen; CN; —COOH; —CONR^{24b}R^{25b}, wherein R^{24b} and R^{25b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30b}R^{31b}, wherein R^{30b} and R^{31b} are independently selected from H, C₁₋₃ alkyl and isopropyl; OH; and R^{18b}—CONH— wherein R^{18b} is selected from C₁₋₃ alkyl and cyclopropyl. Typically, B² is selected from a pyridinyl substituted with a group selected from a cyano and a halogen, such as 1-3 selected from Cl, F, Br, and I. In a particular embodiment B² is selected from a pyridinyl substituted with 1-3 Br, such as one Br. In another particular embodiment B² is selected from a pyridinyl substituted with one Cl and one CN.

In a still further embodiment of the present invention wherein the prodrug has formula II, wherein A² is formula 2b, X¹ is selected from S, SO, SO₂, and O; and B² is selected from B¹ as defined above, R¹ is selected from the group consisting of hydrogen and an in vivo metabolizable group; R² is selected from the group consisting of hydrogen and an in vivo metabolizable group; R³ is selected from the group consisting of hydrogen and an in vivo metabolizable group; with the proviso that at least one in vivo metabolizable group is present in at least one from the group consisting of R¹, R² and R³. In a still further embodiment of the present invention R¹ is an in vivo metabolizable group; R² is hydrogen; and R³ is hydrogen. In a further embodiment of the present invention R¹ is hydrogen; R² is an in vivo metabolizable group; and R³ is hydrogen. In a still further embodiment of the present invention R¹ is hydrogen; R² is hydrogen; and R³ is an in vivo metabolizable group. In a further embodiment of the present invention R¹ is an in vivo metabolizable group; R² is an in vivo metabolizable group; and R³ is hydrogen. In a still further embodiment of the present invention R¹ is hydrogen; R² is an in vivo metabolizable group; and R³ is an in vivo metabolizable group. In a further embodiment of the present invention R¹ is an in vivo metabolizable group; R² is hydrogen; and R³ is an in vivo metabolizable group. In a still further embodiment of the present invention R¹ is an in vivo metabolizable group; R² is an in vivo metabolizable group; and R³ is an in vivo metabolizable group.

In a further embodiment of the present invention the in vivo metabolizable group is independently selected from the group consisting of carbamate, ether, phosphate, sulphate, oxy alkyl phosphate, oxy alkyl sulphate, N-Mannich base, carbonate, amide, ester, N-acylsulphoneamides, sulfonamides, imines, acyloxyalkylamines, phosphates, phosphoroimidates, azoconjugates, carbonyloxymethyl, acethylthioethanol, dithioethanol, cyclosal, Hep-direct, phosphorodiimidatesm ProTide phosphoroimidates, Pro Tide phosphonoimidates, alkoxyalkylmonoeters and acetyl. Typically, the in vivo metabolizable group is independently selected from the group consisting of phosphate, sulphate, oxy C₁₋₆ alkyl phosphate, and oxy C₁₋₆ alkyl sulphate. Each of these in vivo metabolizable groups is considered a single embodiment and may be made the subject of a claim specifically to such in vivo metabolizable group.

In a still further embodiment of the present invention the compound is selected from the group consisting of
5-Bromopyridin-3-yl 3-deoxy-3-[4-(3,4,5-trifluorophenyl)-1H-1,2,3-triazol-1-yl]-6-O-phospho-1-thio-α-D-galactopyranoside,
5-Bromopyridin-3-yl 3-deoxy-3-[4-(3,4,5-trifluorophenyl)-1H-1,2,3-triazol-1-yl]-2-O-phospho-1-thio-α-D-galactopyranoside,
3,4-Dichlorphenyl 3-deoxy-3-[4-(3,4,5-trifluorophenyl)-1H-1,2,3-triazol-1-yl]-2-O-phospho-1-thio-α-D-galactopyranoside,
3,4-Dichlorphenyl 3-deoxy-3-[4-(3,4,5-trifluorophenyl)-1H-1,2,3-triazol-1-yl]-2-O-sulfo-1-thio-α-D-galactopyranoside,
5-Bromopyridin-3-yl 3-Deoxy-3-[4-(3,4,5-trifluorophenyl)-1H-1,2,3-triazol-1-yl]-2-O-sulfo-1-thio-α-D-galactopyranoside,
5-Bromopyridin-3-yl 3-Deoxy-3-[4-(3,4,5-trifluorophenyl)-1H-1,2,3-triazol-1-yl]-6-O-sulfo-1-thio-α-D-galactopyranoside,
5-Bromopyridin-3-yl 3-deoxy-3-[4-(3,4,5-trifluorophenyl)-1H-1,2,3-triazol-1-yl]-6-O-[(phosphonooxy)methyl]-1-thio-α-D-galactopyranoside,
5-Bromopyridin-3-yl 3-deoxy-3-[4-(3,4,5-trifluorophenyl)-1H-1,2,3-triazol-1-yl]-2-O-[(phosphonooxy)methyl]-1-thio-α-D-galactopyranoside,
5-Bromopyridin-3-yl 3-[4-(4-chloro-3,5-difluorophenyl)-1H-1,2,3-triazol-1-yl]-3-deoxy-2-O-[(phosphonooxy)methyl]-1-thio-α-D-galactopyranoside.

In a further aspect the present invention relates to a prodrug compound of the present invention for use as a medicine. In one embodiment the prodrug compound has formula I. In a more preferred embodiment the prodrug compound has formula II.

In a still further aspect the present invention relates to a pharmaceutical composition comprising a compound of formula I or II of the present invention and optionally a pharmaceutically acceptable additive, such as a carrier and/or excipient.

In a further aspect the present invention relates to a compound of formula I or II of the present invention for use in a method for treating a disorder relating to the binding of a galectin-3 to a ligand in a mammal, such as a human. In an embodiment the disorder is selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and conical neovascularization; atherosclerosis; metabolic diseases such as diabetes; type 2 diabetes; insulin resistens; obesity; Diastolic HF; asthma and other interstitial lung diseases, including Hermansky-Pudlak syndrome, mesothelioma; liver disorders, such as non-alcoholic steatohepatitis. A non-limiting group of cancers given as examples of cancers that may be treated, managed and/or prevented by administration of a compound of formula I or II include: colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumors, endometrial cancer, gastric cancer, cancer of the head and neck, chronic lymphoid leukemia, hairy cell leukemia and thyroid cancer. Each of these disorders is considered a single embodiment and may be made the subject of a claim specifically to such disease or disorder.

In a still further aspect the present invention relates to a method for treatment of a disorder relating to the binding of a galectin-3 to a ligand in a mammal, such as a human, wherein a therapeutically effective amount of at least one compound of formula I or II of the present invention is administered to a mammal in need of said treatment. In an embodiment the disorder is selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and conical neovascularization; atherosclerosis; metabolic diseases such as diabetes; type 2 diabetes; insulin resistens; obesity; Diastolic HF; asthma and other interstitial lung diseases, including Hermansky-Pudlak syndrome, mesothelioma; liver disorders, such as non-alcoholic steatohepatitis. Each of these disorders are considered a single embodiment and may be made the subject of a claim specifically to such disease or disorder.

The skilled person will understand that it may be necessary to adjust or change the order of steps in the processes a1-a3 and such change of order is encompassed by the aspects of the process as described above in the reaction schemes and accompanying description of the process steps.

Furthermore, the skilled person will understand that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldipheylsilyl or trimethylsilyl), AcO (acetoxy), TB S (t-butyldimethylsilyl), TMS (trimethylsilyl), PMB (p-methoxybensyl), and tetrahydropyranyl. Suitable protecting groups for carboxylic acid include (C₁₋₆)-alkyl or benzyl esters. Suitable protecting groups for amino include t-butyloxycarbonyl, benzyloxycarbonyl, 2-(trimethylsilyl)-ethoxy-methyl or 2-trimethylsilylethoxycarbonyl (Teoc). Suitable protecting groups for S include S—C(═N)—NH₂, TIPS.

The protection and deprotection of functional groups may take place before or after any reaction in the above-mentioned processes.

Furthermore the skilled person will appreciate, that, in order to obtain compounds of the invention in an alternative, and on some occasions more convenient manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

In a still further embodiment the compound of formula I or II is on free form. “On free form” as used herein means a compound of formula I or II, either an acid form or base form, or as a neutral compound, depending on the substitutents. The free form does not have any acid salt or base salt in addition. In one embodiment the free form is an anhydrate. In another embodiment the free form is a solvate, such as a hydrate.

In a further embodiment the compound of formula I or II is a crystalline form. The skilled person may carry out tests in order to find polymorphs, and such polymorphs are intended to be encompassed by the term “crystalline form” as used herein.

When the compounds and pharmaceutical compositions herein disclosed are used for the above treatment, a therapeutically effective amount of at least one compound is administered to a mammal in need of said treatment.

The term “C₁₋ₓ alkyl” as used herein means an alkyl group containing 1-x carbon atoms, e.g. C₁₋₅ or C₁₋₆, such as methyl, ethyl, propyl, butyl, pentyl or hexyl.

The term “branched C₃₋₆ alkyl” as used herein means a branched alkyl group containing 3-6 carbon atoms, such as isopropyl, isobutyl, tert-butyl, isopentyl, 3-methylbutyl, 2,2-dimethylpropyl, n-hexyl, 2-methylpentyl, 2,2-dimethylbutyl, 2,3-dimethylbutyl.

The term “C₃₋₇ cycloalkyl” as used herein means a cyclic alkyl group containing 3-7 carbon atoms, such as cyclopropyl, cyclobutyl, cyclopentyl, cyclohexyl, cycloheptyl, and 1-methylcyclopropyl.

The term “C₅₋₇ cycloalkyl” as used herein means a cyclic alkyl group containing 5-7 carbon atoms, such as cyclopentyl, cyclohexyl, or cycloheptyl.

The term “Oxo” as used herein means an oxygen atom with double bonds, also indicated as ═O.

The term “CN” as used herein means a nitril.

The term “a five or six membered heteroaromatic ring” as used herein means one five membered heteroaromatic ring or one six membered heteroaromatic ring. The five membered heteroaromatic ring contains 5 ring atoms of which one to four are heteroatoms selected from N, O, and S. The six membered heteroaromatic ring contains 6 ring atoms of which one to five are heteroatoms selected from N, O and S. Examples include thiophene, furan, pyran, pyrrole, imidazole, pyrazole, isothiazole, isooxazole, pyridine, pyrazine, pyrimidine and pyridazine. When such heteroaromatic rings are substituents they are termed thiophenyl, furanyl, pyranyl, pyrrolyl, imidazolyl, pyrazolyl, isothiazolyl, isooxazolyl, pyridinyl, pyrazinyl, pyrimidinyl and pyridazinyl. Also included are oxazoyl, thiazoyl, thiadiazoly, oxadiazoyl, and pyridonyl.

The term “a heterocycle, such as heteroaryl or heterocycloalkyl” as used herein means a heterocycle consisting of one or more 3-7 membered ring systems containing one or more heteroatoms and wherein such ring systems may optionally be aromatic. The term “a heteroaryl” as used herein means a mono or bicyclic aromatic ringsystem containing one or more heteroatoms, such as 1-10, e.g. 1-6, selected from O, S, and N, including but not limited to oxazolyl, oxadiazolyl, thiophenyl, thiadiazolyl, thiazolyl, pyridyl, pyrimidinyl, pyridonyl, pyrimidonyl, quinolinyl, azaquionolyl, isoquinolinyl, azaisoquinolyl, quinazolinyl, azaquinazolinyl, bensozazoyl, azabensoxazoyl, bensothiazoyl, or azabensothiazoyl. The term “a heterocycloalkyl” as used herein means a mono or bicyclic 3-7 membered alifatic heterocycle containing one or more heteroatoms, such as 1-7, e.g. 1-5, selected from O, S, and N, including but not limited to piperidinyl, tetrahydropyranyl, tetrahydrothipyranyl, or piperidonyl.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The treatment may either be performed in an acute or in a chronic way. The patient to be treated is preferably a mammal; in particular, a human being, but it may also include animals, such as dogs, cats, cows, sheep and pigs.

The term “in vivo metabolizable” as used herein means any group that is cleaved of to form an active metabolite when administered to a mammalian subject, which active metabolite has affinity to galectin, such as galectin 3 and is a galectin inhibitor, such as a galectin 3 inhibitor. A typical, in vivo metabolizable group is any one of carbamate, ether, phosphate, sulphate, oxy alkyl phosphate, oxy alkyl sulphate, N-Mannich base, carbonate, amide, ester, N-acylsulphoneamides, sulfonamides, imines, acyloxyalkylamines, phosphates, phosphoroimidates, azoconjugates, carbonyloxymethyl, acethylthioethanol, dithioethanol, cyclosal, Hep-direct, phosphorodiimidatesm ProTide phosphoroimidates, Pro Tide phosphonoimidates, alkoxyalkylmonoeters and acetyl.

The term “a prodrug” as used herein means a compound of formula I or II of the present invention which upon administration to a mammalian body convert into an active metabolite which has high affinity to galectin, such as galectin 3 and is a galectin inhibitor, such as a galectin 3 inhibitor. In particular prodrugs have been developed in which one to three functional group(s) are introduced at selected positions to prepare a prodrug which is charged in the intestine of a mammal, such as a human, due to it's pKa, and in such manner improves solubility for a drug which results in the possibility to give a higher dose. Typical, prodrugs and prodrug strategies are described in Boyapelly, K.; Bonin, M.-A.; Traboulsi, H.; Cloutier, A.; Phaneuf, S. C.; Fortin, D.; Cantin, A. M.; Richter, M. V.; Marsault, E. Synthesis and Characterization of a Phosphate Prodrug of Isoliquiritigenin. J. Nat. Prod. 2017, 80 (4), 879-886. DeGoey, D. A.; Grampovnik, D. J.; Flosi, W. J.; Marsh, K. C.; Wang, X. C.; Klein, L. L.; McDaniel, K. F.; Liu, Y.; Long, M. A.; Kati, W. M.; Molla, A.; Kempf, D. J. Water-Soluble Prodrugs of the Human Immunodeficiency Virus Protease Inhibitors Lopinavir and Ritonavir. J. Med. Chem. 2009, 52 (9), 2964-2970. Rautio, J.; Meanwell, N. A.; Di, L.; Hageman, M. J. The Expanding Role of Prodrugs in Contemporary Drug Design and Development. Nat. Rev. Drug Disc. 2018, 99, 4755. The prodrug may be bioactivated inside or outside a mammalian cell. The preferred prodrugs herein are bioactivated outside a mammalian cell.

The term “a therapeutically effective amount” of a compound of formula I or II of the present invention as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

In a still further aspect the present invention relates to a pharmaceutical composition comprising the compound of formula I or II and optionally a pharmaceutically acceptable additive, such as a carrier or an excipient.

As used herein “pharmaceutically acceptable additive” is intended without limitation to include carriers, excipients, diluents, adjuvant, colorings, aroma, preservatives etc. that the skilled person would consider using when formulating a compound of the present invention in order to make a pharmaceutical composition.

The adjuvants, diluents, excipients and/or carriers that may be used in the composition of the invention must be pharmaceutically acceptable in the sense of being compatible with the compound of formula I or II and the other ingredients of the pharmaceutical composition, and not deleterious to the recipient thereof. It is preferred that the compositions shall not contain any material that may cause an adverse reaction, such as an allergic reaction. The adjuvants, diluents, excipients and carriers that may be used in the pharmaceutical composition of the invention are well known to a person skilled within the art.

As mentioned above, the compositions and particularly pharmaceutical compositions as herein disclosed may, in addition to the compounds herein disclosed, further comprise at least one pharmaceutically acceptable adjuvant, diluent, excipient and/or carrier. In some embodiments, the pharmaceutical compositions comprise from 1 to 99 weight % of said at least one pharmaceutically acceptable adjuvant, diluent, excipient and/or carrier and from 1 to 99 weight % of a compound as herein disclosed. The combined amount of the active ingredient and of the pharmaceutically acceptable adjuvant, diluent, excipient and/or carrier may not constitute more than 100% by weight of the composition, particularly the pharmaceutical composition.

In some embodiments, only one compound as herein disclosed is used for the purposes discussed above.

In some embodiments, two or more of the compounds as herein disclosed are used in combination for the purposes discussed above.

The composition, particularly pharmaceutical composition comprising a compound set forth herein may be adapted for oral, intravenous, topical, intraperitoneal, nasal, buccal, sublingual, or subcutaneous administration, or for administration via the respiratory tract in the form of, for example, an aerosol or an air-suspended fine powder. Therefore, the pharmaceutical composition may be in the form of, for example, tablets, capsules, powders, nanoparticles, crystals, amorphous substances, solutions, transdermal patches or suppositories.

Further embodiments of the process are described in the experimental section herein, and each individual process as well as each starting material constitutes embodiments that may form part of embodiments.

The above embodiments should be seen as referring to any one of the aspects (such as ‘method for treatment’, ‘pharmaceutical composition’, ‘compound for use as a medicament’, or ‘compound for use in a method’) described herein as well as any one of the embodiments described herein unless it is specified that an embodiment relates to a certain aspect or aspects of the present invention.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless other-wise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). This invention includes all modifications and equivalents of the subject matter recited in the aspects or claims presented herein to the maximum extent permitted by applicable law.

The present invention is further illustrated by the following examples that, however, are not to be construed as limiting the scope of protection. The features disclosed in the foregoing description and in the following examples may, both separately and in any combination thereof, be material for realizing the invention indiverse forms thereof.

### Experimental Procedures (Evaluation of Kd Values)

The affinity of Example 1-10 and ref. 1 for galectins were determined by a fluorescence anisotropy assay where the compound was used as an inhibitor of the interaction between galectin and a fluorescein tagged saccharide probe as described Sörme, P., Kahl-Knutsson, B., Huflejt, M., Nilsson, U. J., and Leffler H. (2004) Fluorescence polarization as an analytical tool to evaluate galectin-ligand interactions. Anal. Biochem. 334: 36-47, (Sörme et al., 2004) and Monovalent interactions of Galectin-1 By Salomonsson, Emma; Larumbe, Amaia; Tejler, Johan; Tullberg, Erik.

| | | | Aqueous | |
|---|---|---|---|---|
| | | | Solubility | Galectin-3 |
| Example | Name | Structure | (mg/mL) | Kd (μM) |
| 1 | 5-Bromopyridin-3-yl 3- deoxy-3-[4-(3,4,5- trifluorophenyl)-1H- 1,2,3-triazol-1-yl]-6-O- phospho-1-thio-α-D- galactopyranoside | 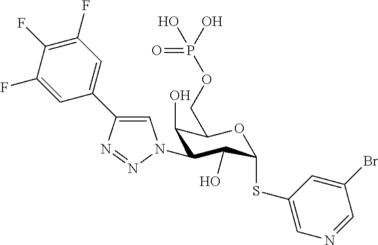 | 8.5 | 35 |
| 2 | 5-Bromopyridin-3-yl deoxy-3-[4-(3,4,5- trifluorophenyl)-1H- 1,2,3-triazol-1-yl]-2-O- phospho-1-thio-α-D- galactopyranoside | 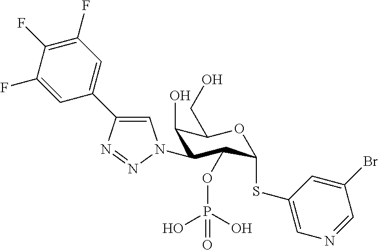 | 9.8 | 0.006 |
| 3 | 3,4-Dichlorphenyl 3- deoxy-3-[4-(3,4,5- trifluorophenyl)-1H- 1,2,3-triazol-1-yl]-2-O- phospho-1-thio-α-D- galactopyranoside | 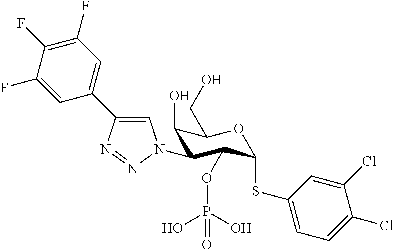 | | 0.007 |
| 4 | 3,4-Dichlorphenyl 3- deoxy-3-[4-(3,4,5- trifluorophenyl)-1H- 1,2,3-triazol-1-yl]-2-O- sulfo-1-thio-α-D- galactopyranoside | 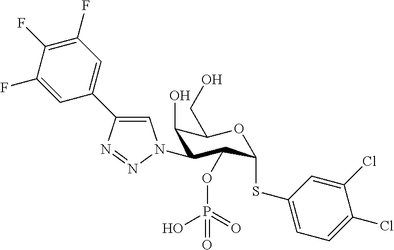 | | 0.014 |
| 5 | 5-Bromopyridin-3-yl 3- Deoxy-3-[4-(3,4,5- trifluorophenyl)-1H- 1,2,3-triazol-1-yl]-2-O- sulfo-1-thio-α-D- galactopyranoside | 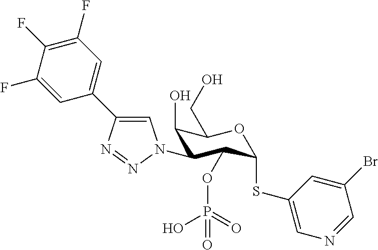 | | 0.004 |
| 6 | 5-Bromopyridin-3-yl 3- Deoxy-3-[4-(3,4,5- trifluorophenyl)-1H- 1,2,3-triazol-1-yl]-6-O- sulfo-1-thio-α-D- galactopyranoside | 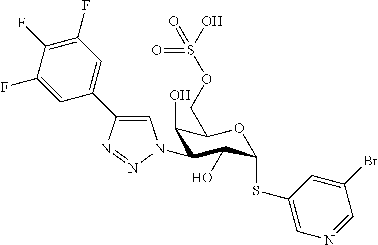 | 9.6 | 27 |
| 7 | 5-Bromopyridin-3-yl 3- deoxy-3-[4-(3,4,5- trifluorophenyl)-1H- 1,2,3-triazol-1-yl]-6-O- [(phosphonooxy)methyl]- 1-thio-α-D- galactopyranoside | 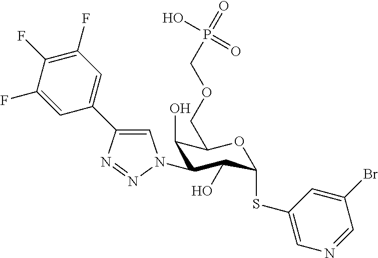 | >10 | 15 |
| 8 | 5-Bromopyridin-3-yl 3- deoxy-3-[4-(3,4,5- trifluorophenyl)-1H- 1,2,3-triazol-1-yl]-2-O- [(phosphonooxy)methyl]- 1-thio-α-D- galactopyranoside | 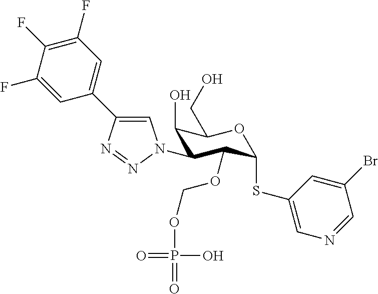 | >10 | 0.01 |
| 9 | 5-Bromopyridin-3-yl 3- [4-(4-chloro-3,5- difluorophenyl)-1H- 1,2,3-triazol-1-yl]-3- deoxy-2-O- [(phosphonooxy)methyl]- 1-thio-α-D- galactopyranoside | 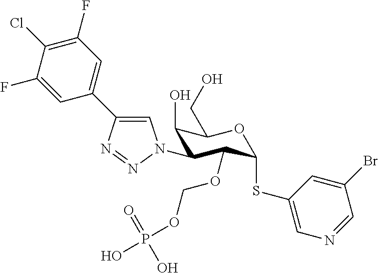 | 0.8 | 0.007 |
| Ref 1 | 5-Bromopyridin-3-yl 3- deoxy-3-[4-(3,4,5- trifluorophenyl)-1H- 1,2,3-triazol-1-yl]-1-thio- α-D-galactopyranoside | 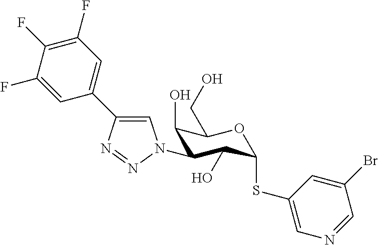 | 0.007 | 0.0248 |
| Ref 2 | 3,4-Dichlorophenyl 3- deoxy-3-[4-(3,4,5- trifluorophenyl)-1H- 1,2,3-triazol-1-yl]-1-thio- α-D-galactopyranoside | 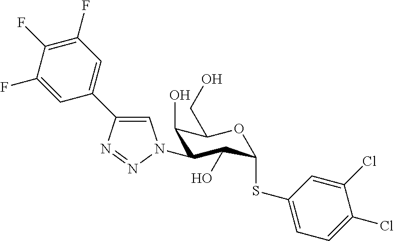 | 0.037 | 0.037 |
| Ref 3 | 5-Bromopyridin-3-yl 3- deoxy-3-[4-(4-chloro-3,5- difluorophenyl)-1H- 1,2,3-triazol-1-yl]-1-thio- α-D-galactopyranoside | 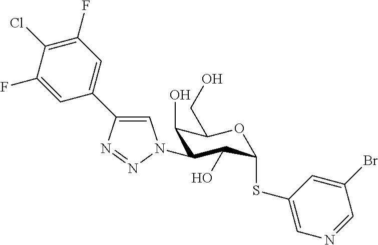 | 0.007 | 0.011 |

Solubility of a drug can limit the maximum possible systemic exposure after per oral (p.o.) administration. This means that there is a correlation between increased dose and the blood concentration up to the solubility limit. Beyond that a drug can crystallise out in the intestine resulting in low and variable exposure. One strategy to enable a drug to be dosed beyond the solubility limit resulting in correlating increased systemic exposure would be to introduce a functional group which both improves the intestinal solubility and is metabolized in the intestine to release the free drug. Examples of such compounds are phosphate ester pro-drugs. In general phosphates are polar, having two negative charges at physiological pH and therefore improve the solubility of a drug. This polarity is also in general limiting the uptake over the intestine resulting in low exposure in plasma of such a pro-drug after p.o. administration. These pro-drugs can be metabolized by phosphates in the intestine to release the drug which is taken up. [Boyapelly, K.; Bonin, M.-A.; Traboulsi, H.; Cloutier, A.; Phaneuf, S. C.; Fortin, D.; Cantin, A. M.; Richter, M. V.; Marsault, E. Synthesis and Characterization of a Phosphate Prodrug of Isoliquiritigenin. J. Nat. Prod. 2017, 80 (4), 879-886.

(2) (1) DeGoey, D. A.; Grampovnik, D. J.; Flosi, W. J.; Marsh, K. C.; Wang, X. C.; Klein, L. L.; McDaniel, K. F.; Liu, Y.; Long, M. A.; Kati, W. M.; Molla, A.; Kempf, D. J. Water-Soluble Prodrugs of the Human Immunodeficiency Virus Protease Inhibitors Lopinavir and Ritonavir. J. Med. Chem. 2009, 52 (9), 2964-2970.

(3) (1) Rautio, J.; Meanwell, N. A.; Di, L.; Hageman, M. J. The Expanding Role of Prodrugs in Contemporary Drug Design and Development. Nat. Rev. Drug Disc. 2018, 99, 47551 There are several examples of how this strategy can be used to increase the maximum dose resulting in dose correlated bioavailability at concentrations high above the drugs aqueous solubility.

Compounds of formula 1 can be pro-drugs with either low (example 1,6 and 7) or high affinity (examples 2-5 and 8) towards the corresponding galectin.

Phosphate esters 7 and 8 are pro-drugs of Ref 1 and have a dose correlated exposure in mice PK far beyond the aqueous solubility of Ref 1.

Ref 1 has a low solubility in water (0.007 mg/mL) and therefore has limited oral availability in some formulations when dosed above 10 mg/kg in mouse pharmacokinetic (PK) experiments. By the introduction of a phosphate group as exemplified by example 7 and 8 the aqueous solubility was improved >1000 fold over Ref 1 and dose correlated exposure was observed after per oral (p.o) administration in mice up to 160 mg/kg.

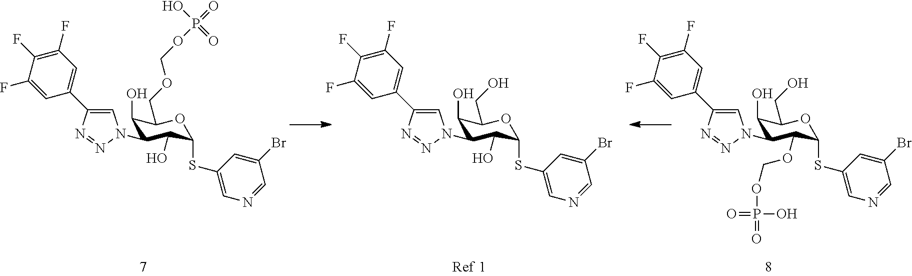

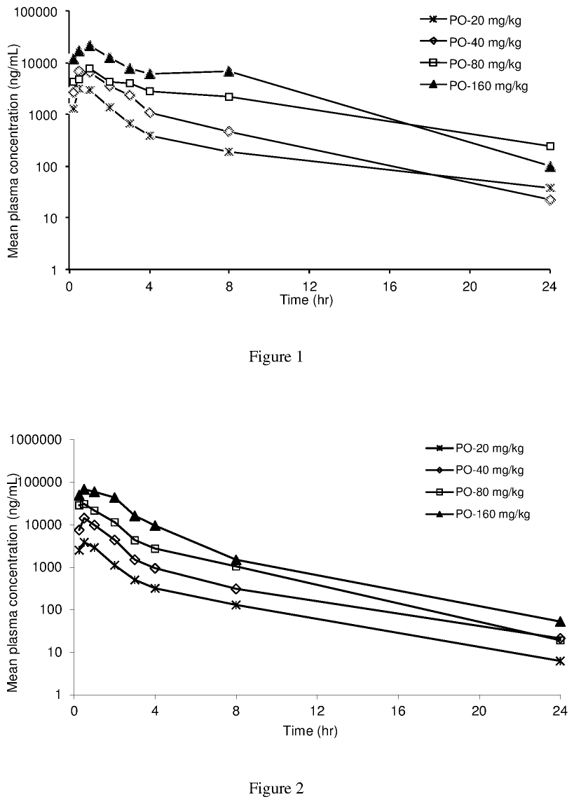

FIG. 1 shows Plasma exposure of Ref 1 after p.o. administration of 7 at 20, 40, 80 and 160 mg/kg in mouse PK experiments.

FIG. 2 shows Plasma exposure of Ref 1 after p.o. administration of 8 at 20, 40, 80 and 160 mg/kg in mouse PK experiments.

Upon metabolic or chemical removal of the prodrug group examples 1-9 the corresponding active metabolite Ref 1-Ref 3 will be formed.

### General Experimental

Nuclear Magnetic Resonance (NMR) spectra were recorded on a 400 MHz Bruker AVANCE LII 500 instrument or a Varian instrument at 400 MHz at 25° C. Chemical shifts are reported in ppm (d) using the residual solvent as internal standard. Peak multiplicities are expressed as follow: s, singlet; d, doublet; dd, doublet of doublets; t, triplet; dt, doublet of triplet; q, quartet; m, multiplet; br s, broad singlet. LC-MS were acquired on an Agilent 1200 HPLC coupled with an Agilent MSD mass spectrometer operating in ES (+) ionization mode. Column: XBridge C18 (4.6×50 mm, 3.5 μm) or SunFire C18 (4.6×50 mm, 3.5 μm). Solvent A water+0.1% TFA and solvent B Acetonitrile+0.1% TFA or solvent A water (10 mM Ammonium hydrogen carbonate) and solvent B Acetonitrile. Wavelength: 254 nM. Alternatively LC-MS were acquired on an Agilent 1100 HPLC coupled with an Agilent MSD mass spectrometer operating in ES (+) ionization mode. Column: Waters symmetry 2.1×30 mm C18 or Chromolith RP-18 2×50 mm Solvent A water+0.1% TFA and solvent B Acetonitrile+0.1% TFA. Wavelength 254 nm.

Preparative HPLC was performed on a Gilson 215. Flow: 25 mL/min Column: XBrige prep C18 10 μm OBD (19×250 mm) column. Wavelength: 254 nM. Solvent A water (10 mM Ammonium hydrogen carbonate) and solvent B Acetonitrile. Alternatively Preparative HPLC were acquired on a Gilson system. Flow: 15 ml/min Column-kromasil 100-5-C18 column Wavelength: 220 nm. Solvent A water+0.1% TFA and solvent B Acetonitrile+0.1% TFA.

The following abbreviations are used

aq: aqueous

### Calcd: Calculated

DCM: dichloromethane

### DIEA: N,N-Diisopropylethylamine

DMAP: 4-dimethylaminopyridine

### DMF: N,N-dimethylformamide

ESI-MS: Electrospray ionization mass spectrometry
EtOAc: ethyl acetate
h: hours
MeCN: acetonitrile
min: minutes
prep. preparative
PE: petroleum ether
rt: Room temperature
TBS: tort-Butyldimethylsilyi
TBA: tetrabutylarnmonium Fluoride
TFA: trifluoroacetic acid
TMS: trimethyl silyl

### UV: Ultraviolet

### Example 1

### 5-Bromopyridin-3-yl 3-deoxy-3-[4-(3,4,5-trifluorophenyl)-1H-1,2,3-triazol-1-yl]-6-O-phospho-1-thio-α-D-galactopyranoside

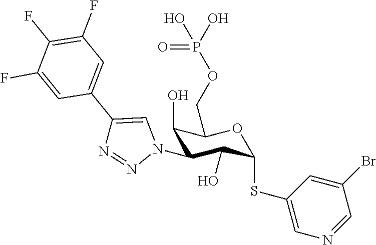

### 5-Bromopyridin-3-yl 6-O-tert-butyldimethylsilyl-3-deoxy-3-[4-(3,4,5-trifluorophenyl)-1H-1,2,3-triazol-1-yl]-1-thio-α-D-galactopyranoside

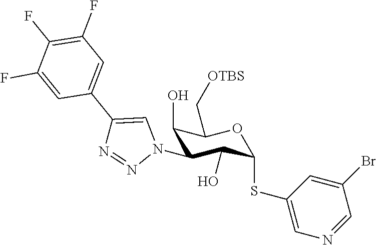

To a solution of 5-Bromopyridin-3-yl 3-deoxy-3-[4-(3,4,5-trifluorophenyl)-1H-1,2,3-triazol-1-yl]-1-thio-α-D-galactopyranoside (200 mg, 0.375 mmol) in DMF (5 mL) was added Imidazolidine (43.9 mg, 0.563 mmol) and TBS-Cl (84.4 mg, 0.563 mmol). The reaction was stirred at rt over 6 h under Na atmosphere. Water (20 mL) and DCM (20 mL) were added. The aqueous phase was extracted with DCM (15 mL×2), the combined organic phases were washed with water (20 mL) and brine (20 mL) and dried over anhydrous sodium sulphate. Removal of the solvent in vacuo gave the title compound which was used next step without further purification.

m/z calcd for [C25H30BrF3N4O4SSi]⁺ [M+H]⁺:647, 649; found: 647, 649.

### Bromopyridin-3-yl 2,4-O-dibenzoyl-6-O-tert-butyldimethylsilyl-3-deoxy-3-[4-(3,4,5-trifluorophenyl)-1H-1,2,3-triazol-1-yl]-1-thio-α-D-galactopyranoside

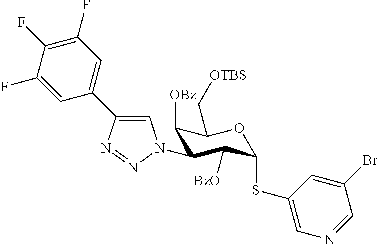

To a solution of 5-Bromopyridin-3-yl 6-O-tert-butyldimethylsilyl-3-deoxy-3-[4-(3,4,5-trifluorophenyl)-1H-1,2,3-triazol-1-yl]-1-thio-α-D-galactopyranoside (243 mg, 0.375 mmol) in pyridine (5.00 mL) was added benzoyl chloride (211 mg, 1.50 mmol). The reaction was stirred at room temperature 6 h under a Na atmosphere. Removal of solvent gave a residue which was purified by column chromatography (PE/EA=4/1) to obtain the title compound (250 mg, 0.292 mmol, yield: 77.9%).

¹H NMR (400 MHz, CDCl3) δ 8.60 (dd, J=6.7, 1.9 Hz, 2H), 8.04-7.99 (m, 2H), 7.97 (t, J=2.0 Hz, 1H), 7.89-7.82 (m, 2H), 7.71-7.63 (m, 2H), 7.58-7.47 (m, 3H), 7.39 (t, J=7.8 Hz, 2H), 7.05 (dd, J=8.0, 6.6 Hz, 2H), 6.35-6.22 (m, 2H), 6.02 (d, J=2.0 Hz, 1H), 5.70-5.57 (m, 1H), 4.82 (t, J=6.5 Hz, 1H), 3.81-3.70 (m, 2H), 0.81 (s, 9H), −0.02 (s, 3H), −0.08 (s, 3H).

### Bromopyridin-3-yl 2,4-O-dibenzoyl-3-deoxy-3-[4-(3,4,5-trifluorophenyl)-1H-1,2,3-triazol-1-yl]-6-O-phospho-1-thio-α-D-galactopyranoside

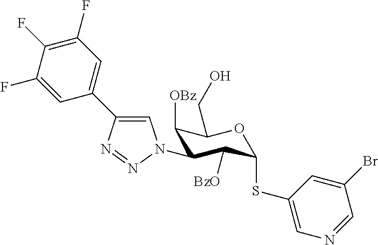

To a solution of Bromopyridin-3-yl 2,4-O-dibenzoyl-6-O-tert-butyldimethylsilyl-3-deoxy-3-[4-(3,4,5-trifluorophenyl)-1H-1,2,3-triazol-1-yl]-1-thio-α-D-galactopyranoside (250 mg, 0.292 mmol) in TBAF-THF (5.00 mL) was stirred at rt for 2 h under a Na atmosphere. Removal of solvent gave a residue which was purified by column chromatography (PE/EA=2/1) to obtain the title compound. (160 mg, 0.216 mmol, yield: 73.9%).

m/z calcd for [C33H24BrF3N4O6S]⁺ [M+H]⁺:741, 743; found: 741.0, 743.0.

¹H NMR (400 MHz, CDCl3) δ 8.56 (s, 1H), 8.48 (s, 1H), 8.08 (s, 1H), 8.02-7.93 (m, 3H), 7.88 (d, J=7.7 Hz, 2H), 7.64-7.53 (m, 2H), 7.50-7.36 (m, 4H), 7.28 (m, 1H), 6.26 (d, J=5.2 Hz, 1H), 6.17 (dd, J=11.5, 5.3 Hz, 1H), 5.40 (d, J=11.8 Hz, 1H), 4.84 (m, 2H), 4.45 (m, 3H).

### Bromopyridin-3-yl 2,4-O-dibenzoyl-3-deoxy-3-[4-(3,4,5-trifluorophenyl)-1H-1,2,3-triazol-1-yl]-6-O-phospho-1-thio-α-D-galactopyranoside

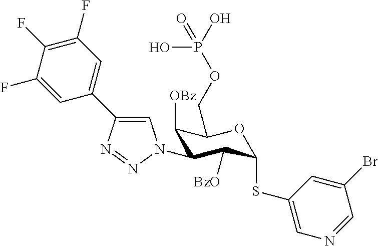

To a solution of bromopyridin-3-yl 2,4-O-dibenzoyl-3-deoxy-3-[4-(3,4,5-trifluorophenyl)-1H-1,2,3-triazol-1-yl]-6-O-phospho-1-thio-α-D-galactopyranoside (90.0 mg, 0.121 mmol) and DMAP (29.7 mg, 0.243 mmol) in DCM (15.00 mL) under Na atmosphere at 0° C. was added DIEA (0.0416 mL, 0.243 mmol) followed by POCl₃ (0.0667 mL, 0.728 mmol). The reaction was stirred for 5 minutes. Water (15 mL) was added and the reaction mixture was stirred for 30 minutes. The reaction mixture was extracted with DCM twice. The combined DCM layer was dried, concentrated and purified by prep.-HPLC to give the title compound (40.0 mg, 0.0487 mmol, yield: 40.1%).

m/z calcd for [C33H25BrF3N4O9PS]⁺ [M+H]⁺:823; found: 823.7.

¹H NMR (400 MHz, CDCl3) δ 8.51 (d, J=10.8 Hz, 2H), 8.28 (s, 1H), 7.90 (s, 1H), 7.85 (d, J=7.2 Hz, 2H), 7.59 (d, J=6.3 Hz, 2H), 7.42 (m, 8H), 7.19 (m, 2H), 6.44 (d, J=4.5 Hz, 1H), 6.39-6.25 (m, 1H), 5.78-5.64 (m, 1H), 5.01 (d, J=9.4 Hz, 1H), 4.81 (d, J=12.0 Hz, 1H), 4.59-4.46 (m, 1H), 4.45-4.31 (m, 1H).

### 5-Bromopyridin-3-yl 3-deoxy-3-[4-(3,4,5-trifluorophenyl)-1H-1,2,3-triazol-1-yl]-6-O-phospho-1-thio-α-D-galactopyranoside

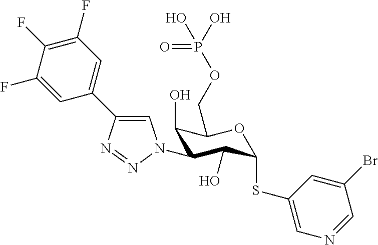

Bromopyridin-3-yl 2,4-O-dibenzoyl-3-deoxy-3-[4-(3,4,5-trifluorophenyl)-1H-1,2,3-triazol-1-yl]-6-O-phospho-1-thio-α-D-galactopyranoside (40.0 mg, 0.0487 mmol) was dissolved in 7M NH₃ in MeOH (15 mL) solution and the reaction was stirred at rt for 24 h. Solvent was removed and the residue was purified by prep.-HPLC to give the title compound (15.0 mg, 0.0245 mmol, yield: 50.2%) as a white solid.

m/z calcd for [C19H17BrF3N4O7PS]⁻ [M−H]⁻:611, 613; found: 610.9, 612.9.

¹H NMR (400 MHz, MeOD) δ 8.66-8.59 (m, 2H), 8.55 (d, J=1.8 Hz, 1H), 8.23 (s, 1H), 7.72-7.59 (m, 2H), 5.95 (d, J=5.1 Hz, 1H), 5.14 (d, J=9.3 Hz, 1H), 4.97 (dd, J=11.4, 5.3 Hz, 1H), 4.79 (m, 1H), 4.49-4.39 (m, 1H), 3.94-3.80 (m, 1H), 3.47 (dd, J=11.7, 5.7 Hz, 1H).

### Example 2

### 5-Bromopyridin-3-yl 3-deoxy-3-[4-(3,4,5-trifluorophenyl)-1H-1,2,3-triazol-1-yl]-2-O-phospho-1-thio-α-D-galactopyranoside

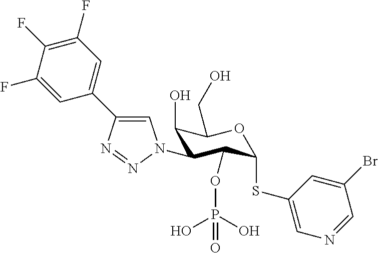

### 5-Bromopyridin-3-yl 4,6-O-benzyliden-3-deoxy-3-[4-(3,4,5-trifluorophenyl)-1H-1,2,3-triazol-1-yl]-2-O-phosopho-1-thio-α-D-galactopyranoside

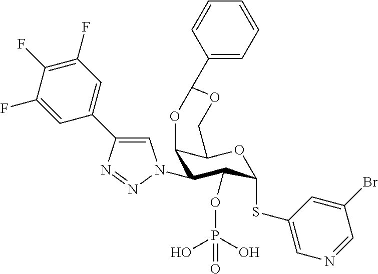

To a solution of 5-Bromopyridin-3-yl 4,6-O-benzyliden-3-deoxy-3-[4-(3,4,5-trifluorophenyl)-1H-1,2,3-triazol-1-yl]-1-thio-α-D-galactopyranoside (200 mg, 0.322 mmol) and DMAP (78.6 mg, 0.644 mmol) in DCM (15.00 mL) under Na atmosphere at 0° C. was added DIEA (1.38 mL, 8.05 mmol) followed by POCl₃ (0.147 mL, 1.61 mmol) dropwise. After TLC showed no remaining starting material the solvent was removed under vacuo at r.t and ammonium hydroxide (15 mL) was added, white solid precipitated out which was collected by filtration and was used directly to the next step without further purification.

m/z calcd for [C26H21BrF3N4O7PS]⁻ [M−H]⁻:699, 701; found: 699, 701.

### 5-Bromopyridin-3-yl 3-deoxy-3-[4-(3,4,5-trifluorophenyl)-1H-1,2,3-triazol-1-yl]-2-O-phospho-1-thio-α-D-galactopyranoside

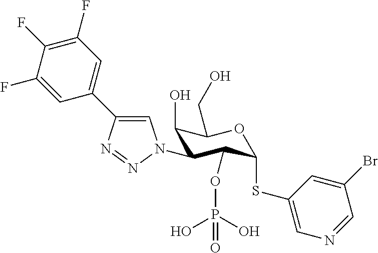

5-Bromopyridin-3-yl 4,6-O-benzyliden-3-deoxy-3-[4-(3,4,5-trifluorophenyl)-1H-1,2,3-triazol-1-yl]-2-O-phospho-1-thio-α-D-galactopyranoside (220 mg, 0.314 mmol) was dissolved in 10% TFA in DCM and the reaction was stirred for 3 h. The solvent was removed and the residue was purified by Prep.-HPLC to give the title compound. (63.4 mg, 0.103 mmol, yield: 33.0%).

m/z calcd for [C19H17BrF3N4O7PS]⁻ [M−H]⁻:611, 613; found: 610.8, 613.8. ¹H NMR (400 MHz, MeOD) δ 8.71 (d, J=1.7 Hz, 1H), 8.63 (s, 1H), 8.55 (d, J=2.0 Hz, 1H), 8.38 (t, J=1.9 Hz, 1H), 7.66 (dd, J=8.8, 6.7 Hz, 2H), 6.24 (d, J=5.3 Hz, 1H), 5.43-5.31 (m, 1H), 5.13 (dd, J=11.5, 2.7 Hz, 1H), 4.55 (t, J=5.9 Hz, 1H), 4.22 (s, 1H), 3.77-3.65 (m, 2H).

### Example 3

### 3,4-Dichlorophenyl 3-deoxy-3-[4-(3,4,5-trifluorophenyl)-1H-1,2,3-triazol-1-yl]-2-O-phospho-1-thio-α-D-galactopyranoside

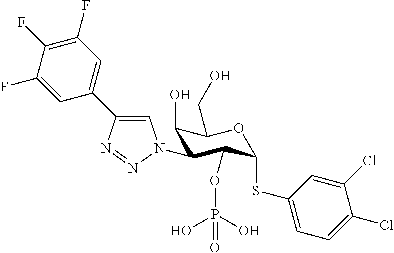

### 3,4-Dichlorophenyl 4,6-O-benzylidene-3-deoxy-3-[4-(3,4,5-trifluorophenyl)-1H-1,2,3-triazol-1-yl]-1-thio-α-D-galactopyranoside

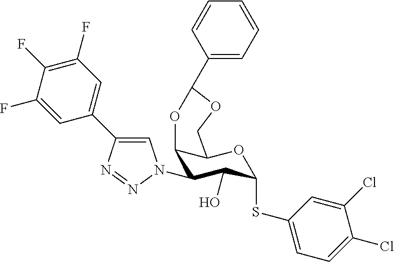

To a solution of 3,4-Dichlorophenyl 4,6-O-benzylidene-3-deoxy-3-[4-(3,4,5-trifluorophenyl)-1H-1,2,3-triazol-1-yl]-1-thio-α-D-galactopyranoside (605 mg, 1,158 mmol) in dry MeCN (24 ml), Bn(OMe)₂ (0.87 mL, 5.791 mmol) and camphorsulfonic acid (30 mg, 0.116 mmol) dissolved in dry MeCN (1 mL) was added. The transparent yellow mixture was left stirring at r.t. After 20 min the reaction mixture became turbid of white precipitate. After 2 h the reaction was stopped by evaporation of solvent. The reaction crude was dissolved in EtOH (180 mL) under heating and left at r.t. for 36 h to crystalize. The crystals formed were filtered off and dried under vacuum to obtain the title compound (247 mg, 35%) as amorphous white solid.

¹H NMR ((CD3)₂SO, 400 MHz): δ 8.85 (s, 1H, Ph), 7.84-7.76 (m, 3H, Ph), 7.63 (d, J=8.6 Hz, 1H, Ph), 7.52 (dd, J=8.6, 2.1 Hz, 1H, Ph), 7.39-7.30 (m, 5H, Ph), 6.17 (d, J=5.3 Hz, 1H, H-1), 6.10 (d, J=4.8 Hz, 1H, OH-2), 5.57 (s, 1H, CH), 5.09 (dd, J=11.0, 3.1 Hz, 1H, H-3), 4.90 (m, 1H, H-2), 4.56 (d, J=2.9 Hz, 1H, H-4), 4.26 (s, 1H, H-5), 4.13 (d, J=12.5 Hz, 1H, H-6), 3.96 (d, J=12.5 Hz, 1H, H-6). ¹³C NMR ((CD3)₂SO, 100 MHz): δ 137.8, 135.1, 131.7, 131.5, 130.9, 130.7, 129.4, 128.7, 128.0, 125.9, 122.8, 109.5, 109.3, 99.4, 88.2, 74.1, 68.3, 64.2, 63.1, 61.5. HRMS calculated for [C₂₇H₂₁F₃Cl₂N₃O₄S]⁺, 610.0582; found: 610.0575.

### 3,4-Dichlorophenyl-yl 3-deoxy-3-[4-(3,4,5-trifluorophenyl)-1H-1,2,3-triazol-1-yl]-2-O-phospho-1-thio-α-D-galactopyranoside

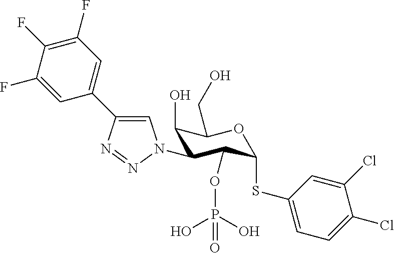

Imidazole and 3,4-dichlorophenyl 4,6-O-benzylidene-3-deoxy-3-[4-(3,4,5-trifluorophenyl)-1H-1,2,3-triazol-1-yl]-1-thio-α-D-galactopyrano side was rendered anhydrous by co-evaporation with toluene. PCl₃ (0.10 mL, 1.18 mmol) in dry MeCN:DCM 1:1 (4 mL) was cannulated into a stirring solution of imidazole (380 mg, 5.50 mmol) in dry MeCN:DCM 1:1 (6 mL) under N₂-atmosphere. The reaction mixture turned white/light green and turbid. Directly following, Et₃N (0.44 mL, 3.15 mmol) in dry MeCN:DCM 1:1 (4 mL) was cannulated into the stirring solution. 3,4-Dichlorophenyl 4,6-O-benzylidene-3-deoxy-3-[4-(3,4,5-trifluorophenyl)-1H-1,2,3-triazol-1-yl]-1-thio-α-D-galactopyrano side (240 mg, 0.39 mmol) dissolved in dry MeCN:DCM 1:1 (50+10 mL) and was slowly cannulated into the stirring solution over 25 min. The reaction mixture was stirred at 0° C. for 45 min before pyridine:H₂O 4:1 (50 mL) was added. TLC showed complete consumption of starting material. The reaction crude was concentrated and purified by column chromatography (SiO₂, DCM:MeOH 10:1 with 1% Et₃N). The phosphonate was dissolved in dry pyridine (9 mL). TMS-Cl (0.5 mL, 3.93 mmol) was added and the reaction left stirring at r.t. for 5 min before iodine (205 mg, 0.79 mmol) in dry pyridine (1 mL) was added. After stirring for 10 min, water (0.35 mL) was added and the solvent was evaporated. The reaction crude was purified by repeated column chromatography (SiO₂, DCM:MeOH:H₂O 10:1:0->65:35:1). The resulting solid material was dissolved in HOAc (70% aq., 20 mL) and left stirring at 40° C. After 30 h the solvent was evaporated and the crude purified by column chromatography (SiO₂, DCM:MeOH:H₂O 5:1:0->65:35:1) to give the title compound (48 mg, 20%) as amorphous white solid.

¹H NMR (MeOD, 400 MHz): δ 8.55 (s, 1H, Ph), 7.64 (dd, J=9.0, 6.8 Hz, 2H, Ph) 7.57 (dd, J=8.4, 2.2 Hz, 1H, Ph), 7.49 (d, J=8.4 Hz, 1H, Ph), 6.08 (d, J=5.4 Hz, 1H, H-1), 5.41 (m, 1H, H-2), 5.17 (dd, J=11.5, 2.9 Hz, 1H, H-3) 4.57 (t, J=6.3 Hz, 1H, H-5), 4.23 (m, 1H, H-4), 3.73 (m, 2H, H-6). ¹³C NMR (MeOD, 100 MHz): δ 135.3, 135.3, 133.6, 133.0, 131.9, 123.2, 111.0, 110.8, 89.2, 73.6, 71.0, 69.9, 64.3, 64.2, 64.1. HRMS calculated for [C₂₀H₁₈F₃Cl₂N₃O₇PS]⁺, 601.9932; found: 601.9934.

### Example 4

### 3,4-Dichlorphenyl 3-Deoxy-3-[4-(3,4,5-trifluorophenyl)-1H-1,2,3-triazol-1-yl]-2-O-sulfo-1-thio-α-D-galactopyranoside

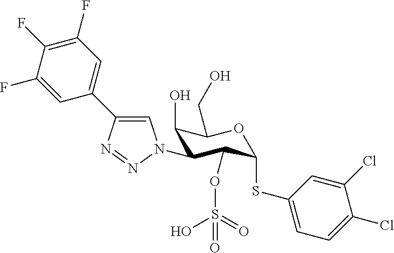

3,4-Dichlorophenyl 4,6-O-benzylidene-3-deoxy-3-[4-(3,4,5-trifluorophenyl)-1H-1,2,3-triazol-1-yl]-1-thio-α-D-galactopyranoside (225 mg, 0.37 mmol) and sulfur trioxide-triethylamine complex (560 mg, 3.82 mmol) were dissolved in dry DMF (20 mL) and stirred at 50° C. After 30 h the solvents were evaporated of solvent and the reaction crude purified by column chromatography (SiO₂, EtOAc:Heptane 9:1->1:0). Partial desulfation during evaporation of solvent after column chromatography was observed. The crude was dissolved in HOAc (90% aq., 30 mL) and left stirring at 40° C. for 22 h followed by evaporation of the solvent. The crude was purified by column chromatography (SiO₂, EtOAc:Hep 1:1->EtOAc:Hep 1:0) to give the title compound (48 mg, 22%) as amorphous white solid.

¹H NMR (MeOD, 400 MHz): δ 8.56 (s, 1H, Ph), 7.83 (d, J=2.1 Hz, 1H, Ph), 7.62 (dd, J=9.0, 6.3 Hz, 2H, Ph), 7.57 (dd, J=8.3, 2.1 Hz, 1H, Ph), 7.48 (d, J=8.3 Hz, 1H, Ph), 6.24 (d, J=5.3 Hz, 1H, H-1), 5.48 (dd, J=11.7, 5.3 Hz, 1H, H-2), 5.13 (dd, J=11.7, 2.9 Hz, 1H, H-3), 4.56 (t, J=6.1 Hz, 1H, H-5), 4.26 (d, J=2.8 Hz, 1H, H-4), 4.12 (d, J=7.2 Hz, 1H, H-6), 4.08 (d, J=7.2 Hz, 1H, H-6).

### Example 5

### 5-Bromopyridin-3-yl 3-Deoxy-3-[4-(3,4,5-trifluorophenyl)-1H-1,2,3-triazol-1-yl]-2-O-sulfo-1-thio-α-D-galactopyranoside

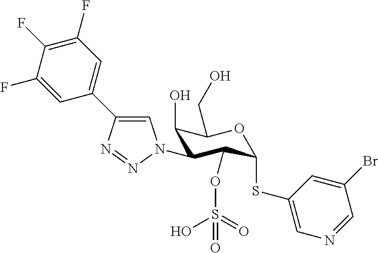

### 5-Bromopyridin-3-yl 4,6-O-benzylidene-3-deoxy-3-[4-(3,4,5-trifluorophenyl)-1H-1,2,3-triazol-1-yl]-1-thio-α-D-galactopyranoside

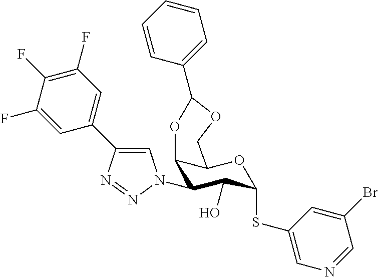

To a stirred solution of 5-bromopyridin-3-yl 3-deoxy-3-[4-(3,4,5-trifluorophenyl)-1H-1,2,3-triazol-1-yl]-1-thio-α-D-galactopyranoside (200 mg, 0.375 mmol) in DMF (10 mL) was added benzaldehyde dimethyl acetal (285 mg, 1.88 mmol) followed by D(+)-10-Camphorsulfonic acid (17.4 mg, 0.0750 mmol). The resulting mixture was stirred at 50° C. for 3 hours under vacuum. LCMS showed full consumption of starting material and a resulting product spot. The solution was added to aq NaHCO₃ (100 mL) dropwise with vigorous stirring. Then it was filtrated and washed with water to get 220 mg (94.4%) of the title compound as a white solid.

m/z calcd for [C₂₆H₂₀BrF₃N₄O₄S]⁺ [M+H]⁺: 621.0, 623.0; found: 621.0, 623.0.

¹H NMR (400 MHz, DMSO) δ 8.86 (s, 1H), 8.67 (d, J=1.5 Hz, 1H), 8.62 (d, J=1.9 Hz, 1H), 8.28 (s, 1H), 7.87-7.72 (m, 2H), 7.35 (dt, J=9.8, 4.9 Hz, 5H), 6.25 (d, J=5.2 Hz, 1H), 6.17 (d, J=4.8 Hz, 1H), 5.57 (s, 1H), 5.12 (dd, J=11.3, 3.2 Hz, 1H), 4.91 (dd, J=10.9, 5.4 Hz, 1H), 4.57 (d, J=2.9 Hz, 1H), 4.29 (s, 1H), 4.12 (d, J=12.2 Hz, 1H), 3.92 (d, J=12.7 Hz, 1H).

### 5-Bromopyridin-3-yl 4,6-O-benzylidene-3-deoxy-3-[4-(3,4,5-trifluorophenyl)-1H-1,2,3-triazol-1-yl]-2-O-sulfo-1-thio-α-D-galactopyranoside

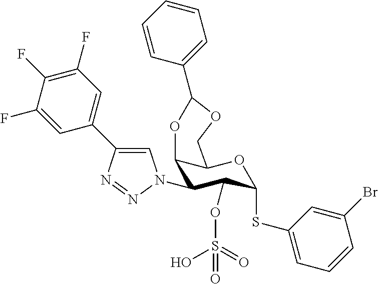

5-Bromopyridin-3-yl 4,6-O-benzylidene-3-deoxy-3-[4-(3,4,5-trifluorophenyl)-1H-1,2,3-triazol-1-yl]-1-thio-α-D-galactopyranoside (150.0 mg, 0.24 mmol) and sulfur trioxide trimethylamine complex(672 mg, 4.83 mmol) were dissolved in anhydrous DMF (10.0 mL), the reaction was stirred at rt o/n under Na. LCMS indicated almost full consumption of starting material. The reaction mixture was purified by Prep.-HPLC to give the title compound (65.0 mg, 0.093 mmol, yield: 38%) obtained as white solid.

m/z calcd for [C₂₆H₂₀BrF₃N₄O₇S₂]⁻ [M−H]−: 699.0, 701.0; found: 699.0, 701.0.

### 5-Bromopyridin-3-yl 3-Deoxy-3-[4-(3,4,5-trifluorophenyl)-1H-1,2,3-triazol-1-yl]-2-O-sulfo-1-thio-α-D-galactopyranoside

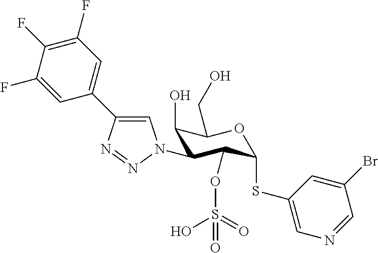

5-Bromopyridin-3-yl 4,6-O-benzylidene-3-deoxy-3-[4-(3,4,5-trifluorophenyl)-1H-1,2,3-triazol-1-yl]-2-O-sulfo-1-thio-α-D-galactopyranoside (20.0 mg, 0.0285 mmol) was dissolved in CH₃CO₂H (1.60 mL) and H₂O (0.400 mL). The reaction was stirred at 50° C. for 4 h. TLC indicated full consumption of starting material. Solvent was removed and the residue was purified by Prep.-HPLC to give the title compound (3.20 mg, 0.005 mmol, yield: 18.3%).

m/z calcd for [C₁₉H₁₆BrF₃N₄O₇S₂]⁻ [M−H]⁻: 611.0, 613.0; found: 611.0, 613.0.

¹H NMR (400 MHz, MeOD) δ 8.71 (s, 1H), 8.56 (s, 2H), 8.35 (s, 1H), 7.75-7.49 (m, 2H), 6.26 (d, J=5.3 Hz, 1H), 5.49 (dd, J=11.7, 5.3 Hz, 1H), 5.16 (dd, J=11.8, 2.6 Hz, 1H), 4.56 (dd, J=13.5, 7.6 Hz, 1H), 4.27 (s, 1H), 3.71 (d, J=6.0 Hz, 2H).

### Example 6

### 5-Bromopyridin-3-yl 3-Deoxy-3-[4-(3,4,5-trifluorophenyl)-1H-1,2,3-triazol-1-yl]-6-O-sulfo-1-thio-α-D-galactopyranoside

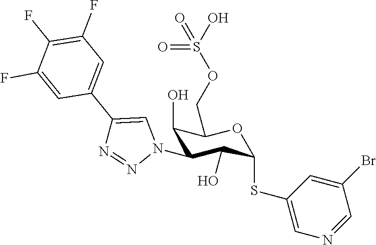

To a solution of 5-Bromopyridin-3-yl 3-deoxy-3-[4-(3,4,5-trifluorophenyl)-1H-1,2,3-triazol-1-yl]-1-thio-α-D-galactopyranoside (100 mg, 0.188 mmol) and sulfur trioxide trimethylamine (52.0 mg, 0.374 mmol) in DMF (5.00 mL) was stirred at room temperature overnight under a nitrogen atmosphere. Removal of solvent gave a residue which was purified by column chromatography to obtain the title compound (56 mg, yield: 48.7%).

m/z calcd for [C₁₉H₁₆Br₁₃F₃N₄O₇S₂]⁺ [M+H]⁺:614; found: 614.

¹H NMR (400 MHz, MeOD) δ 8.67 (d, J=1.8 Hz, 1H), 8.59-8.52 (m, 2H), 8.28 (t, J=2.0 Hz, 1H), 7.66 (dd, J=8.9, 6.7 Hz, 2H), 5.89 (d, J=5.3 Hz, 1H), 5.03 (dd, J=11.4, 2.8 Hz, 1H), 4.94 (dd, J=11.4, 5.3 Hz, 1H), 4.75 (t, J=6.7 Hz, 1H), 4.26 (d, J=1.9 Hz, 1H), 4.12 (ddd, J=59.7, 10.6, 6.4 Hz, 2H).

### Example 7

### 5-Bromopyridin-3-yl 3-deoxy-3-[4-(3,4,5-trifluorophenyl)-1H-1,2,3-triazol-1-yl]-6-O-[(phosphonooxy)methyl]-1-thio-α-D-galactopyranoside

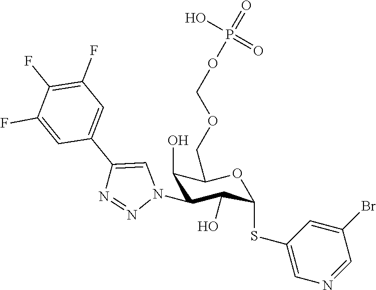

### 5-Bromopyridin-3-yl 6-O-tert-butyldimethylsilyl-3-deoxy-3-[4-(3,4,5-trifluorophenyl)-1H-1,2,3-triazol-1-yl]-1-thio-α-D-galactopyranoside

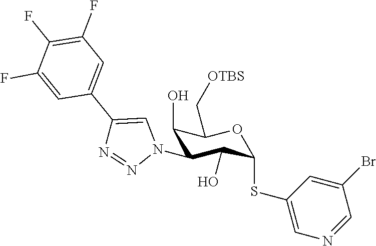

5-Bromopyridin-3-yl 3-deoxy-3-[4-(3,4,5-trifluorophenyl)-1H-1,2,3-triazol-1-yl]-1-thio-α-D-galactopyranoside (GB1211, 10.0 g, 18.7 mmol), imidazole (29 mmol, 2.0 g) and TBDMS-Cl (1.1 eq., 20.6 mmol, 3.2 g) were stirred at room temperature in DMF (40 ml). TLC (EtOAc) after 15 min was OK. Water (80 ml) was added under vigorous stirring, the precipitate was isolated by filtration, washed with water and then dried on a rotary evaporator until constant weight. The title compound weighed 12.2 g (100% yield); ¹H NMR (400 MHz, DMSO-d₆) δ 8.80 (s, 1H), 8.65 (d, J=1.7 Hz, 1H), 8.56 (d, J=2.0 Hz, 1H), 8.20 (t, J=1.9 Hz, 1H), 7.85 (m, 2H), 6.13 (d, J=5.2 Hz, 1H), 5.97 (d, J=4.8 Hz, 1H), 5.54 (d, J=6.5 Hz, 1H), 4.87 (dd, J=11.3, 2.5 Hz, 1H), 4.77 (dt, J=11.0, 5.0 Hz, 1H), 4.24 (t, J=5.7 Hz, 1H), 4.03 (d, J=4.5 Hz, 1H), 3.70 (dd, J=10.8, 4.6 Hz, 1H), 3.57 (dd, J=10.7, 7.0 Hz, 1H), 0.78 (s, 9H), −0.03 (s, 3H), −0.04 (s, 3H).

### 5-Bromopyridin-3-yl 2,4-di-O-benzoyl-6-O-tert-butyldimethylsilyl-3-deoxy-3-[4-(3,4,5-trifluorophenyl)-1H-1,2,3-triazol-1-yl]-1-thio-α-D-galactopyranoside

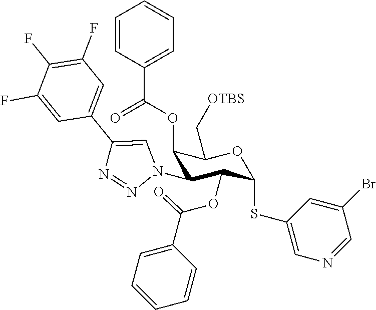

To 5-bromopyridin-3-yl 6-O-tert-butyldimethylsilyl-3-deoxy-3-[4-(3,4,5-trifluorophenyl)-1H-1,2,3-triazol-1-yl]-1-thio-α-D-galactopyranoside (12.2 g, 18.7 mmol) in pyridine (70 ml) benzoyl chloride (3 eq., 56.4 mmol, 6.61 ml) was added. The reaction is slow, and the mixture was stirred for 20 h at 35° C. TLC (PE/EtOAc: 3/1) indicated complete reaction. Water (160 ml) was added, which resulted in precipitation of sticky material, the mixture was decanted, and the sticky residue was stirred in EtOH (120 ml), acetic acid (4 ml) and water (20 ml), which resulted in crystallization. The precipitate was isolated by filtration, washed with 33% aqueous MeOH, and dried to afford the title compound (14.0 g, 87% yield); ¹H NMR (400 MHz, Chloroform-d) δ 8.65-8.57 (m, 2H), 8.03 (d, J=7.3 Hz, 2H), 7.99 (d, J=1.9 Hz, 1H), 7.87 (d, J=7.5 Hz, 2H), 7.72-7.64 (m, 2H), 7.62-7.48 (m, 3H), 7.40 (t, J=7.8 Hz, 2H), 7.12-6.98 (m, 2H), 6.34-6.26 (m, 2H), 6.07-6.01 (m, 1H), 5.70-5.60 (m, 1H), 4.82 (t, J=6.5 Hz, 1H), 3.83-3.70 (m, 2H), 0.82 (s, 9H), −0.01 (s, 3H), −0.07 (s, 3H).

### 5-bromopyridin-3-yl 2,4-di-O-benzoyl-3-deoxy-3-[4-(3,4,5-trifluorophenyl)-1H-1,2,3-triazol-1-yl]-1-thio-α-D-galactopyranoside

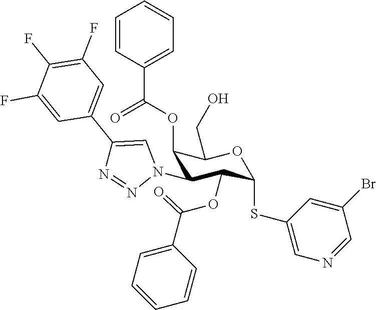

5-Bromopyridin-3-yl 2,4-di-O-benzoyl-6-O-tert-butyldimethylsilyl-3-deoxy-3-[4-(3,4,5-trifluorophenyl)-1H-1,2,3-triazol-1-yl]-1-thio-α-D-galactopyranoside (6.5 g, 7.59 mmol) was dissolved in THF (45 ml) and acetic acid (4.0 eq., 1.8 ml) followed by addition of TBAF (3.0 eq., 1 M solution, 22.75 mmol, 22.75 ml). The mixture was stirred at 40° C. for 30 min, it was then cooled and concentrated to one third of the volume at <30° C. and applied directly onto a silica column conditioned in PE/EtOAc 4/1. The column was eluted with PE/EtOAc 2/1-1/1-0/1 and gave the title compound (4.65 g, 83% yield); ¹H NMR (400 MHz, DMSO-d₆) δ 8.88 (s, 1H), 8.66 (d, J=1.6 Hz, 1H), 8.63 (d, J=1.9 Hz, 1H), 8.32 (t, J=1.7 Hz, 1H), 7.94 (d, J=7.8 Hz, 2H), 7.77 (d, J=7.9 Hz, 2H), 7.70 (t, J=7.3 Hz, 1H), 7.67-7.53 (m, 5H), 7.47 (t, J=7.8 Hz, 2H), 6.56 (d, J=5.5 Hz, 1H), 6.38 (dd, J=11.6, 5.5 Hz, 1H), 5.93 (s, 1H), 5.89 (dd, J=11.6, 2.8 Hz, 1H), 5.08 (t, J=5.3 Hz, 1H), 4.78 (t, J=6.4 Hz, 1H), 3.56-3.40 (m, 2H). The faster moving (TLC) impurity, formed by migration of a benzoate group (from 4- to 6-position) during the desilylation reaction was also present in the chromatographed product (2%).

### 5-Bromopyridin-3-yl 2,4-di-O-benzoyl-3-deoxy-3-[4-(3,4,5-trifluorophenyl)-1H-1,2,3-triazol-1-yl]-6-O-[(phosphonooxy)methyl]-1-thio-α-D-galactopyranoside

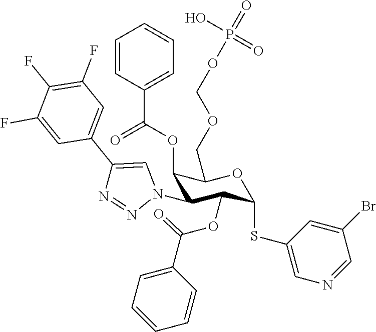

Anhydrous HCl was slowly bubbled through a mixture of 5-bromopyridin-3-yl 2,4-di-O-benzoyl-3-deoxy-3-[4-(3,4,5-trifluorophenyl)-1H-1,2,3-triazol-1-yl]-1-thio-α-D-galactopyranoside (612 mg, 0.825 mmol) and paraformaldehyde (2.0 eq., 1.65 mmol, 49.6 mg) in dichloromethane (16.0 ml) for 3 hours at 0° C. The mixture was diluted with dry THF (6 ml), dried (MgSO4), filtered and concentrated. Di-tetrabutylammonium phosphate (959 mg, 1.65 mmol) in THF (2.0 ml) was added, the mixture was stirred for 30 min and then concentrated. The crude material was purified by reversed phase chromatography, eluting with 30-80% acetonitrile in water (25 mM NH₄OAc, pH 5.2) to afford the title compound as the mono tetrabutylammonium salt (378 mg, 42% yield); ESI-MS m/z calcd for [C₃₄H₂₇BrF₃N₄O₁₀PS]⁺ (M+H)⁺: 851.0; found: 851.1, ¹H NMR (400 MHz, Methanol-d4) δ 8.68-8.62 (m, 1H), 8.59 (s, 2H), 8.39-8.33 (m, 1H), 8.01 (d, J=7.2 Hz, 2H), 7.84 (d, J=7.3 Hz, 2H), 7.64 (t, J=7.5 Hz, 1H), 7.58 (t, J=7.4 Hz, 1H), 7.51 (t, J=7.7 Hz, 2H), 7.47-7.37 (m, 4H), 6.50 (dd, J=11.6, 5.6 Hz, 1H), 6.42 (d, J=5.6 Hz, 1H), 6.11-6.01 (m, 1H), 5.85 (dd, J=11.6, 2.7 Hz, 1H), 5.24-5.16 (m, 1H), 5.08 (dd, J=9.4, 5.5 Hz, 1H), 5.03 (dd, J=9.6, 5.4 Hz, 1H), 3.94 (dd, J=11.0, 4.6 Hz, 1H), 3.79 (dd, J=10.8, 7.5 Hz, 1H), 3.28-3.19 (m, 8H), 1.72-1.60 (m, 8H), 1.49-1.35 (m, 8H), 1.03 (t, J=7.4 Hz, 12H).

### 5-Bromopyridin-3-yl 3-deoxy-3-[4-(3,4,5-trifluorophenyl)-1H-1,2,3-triazol-1-yl]-6-O-[(phosphonooxy)methyl]-1-thio-α-D-galactopyranoside

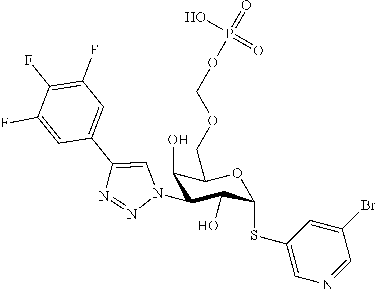

Ammonium hydroxide (28% aqueous ammonia, 47.4 mmol, 5.0 ml) was added to (5-bromopyridin-3-yl) 2,4-di-O-benzoyl-3-deoxy-3-[4-(3,4,5-trifluorophenyl)-1H-1,2,3-triazol-1-yl]-6-O-[(phosphonooxy)methyl]-1-thio-α-D-galactopyranoside (565 mg, 0.517 mmol) in MeOH (5.0 ml) and THF (5.0 ml). The mixture was heated 6 hours at 60° C., then cooled to rt and concentrated. The crude material was purified by reversed phase chromatography, eluting with 10-50% acetonitrile in water (25 mM NH₄OAc) to afford the title compound as the ammonium salt (225 mg, 66% yield); HPLC 99%, ESI-MS m/z calcd for [C₂₀H₁₉BrF₃N₄O₈PS]⁺ (M+H)⁺: 643.0; found: 643.1, ¹H NMR (400 MHz, Methanol-d4) δ 8.68-8.63 (m, 1H), 8.58 (s, 1H), 8.58-8.54 (m, 1H), 8.32 (t, J=1.8 Hz, 1H), 7.66 (dd, J=8.8, 6.7 Hz, 2H), 5.88 (d, J=5.3 Hz, 1H), 5.15 (dd, J=8.6, 5.8 Hz, 1H), 5.05 (dd, J=11.4, 2.7 Hz, 1H), 4.99-4.89 (m, 2H), 4.63 (t, J=6.1 Hz, 1H), 4.27 (d, J=2.1 Hz, 1H), 4.01 (dd, J=9.9, 6.1 Hz, 1H), 3.64 (dd, J=9.8, 6.9 Hz, 1H).

### Example 8

### 5-Bromopyridin-3-yl 3-deoxy-3-[4-(3,4,5-trifluorophenyl)-1H-1,2,3-triazol-1-yl]-2-O-[(phosphonooxy)methyl]-1-thio-α-D-galactopyranoside

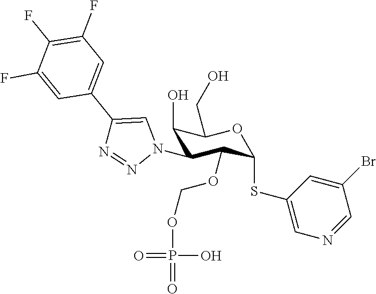

### 5-Bromopyridin-3-yl 4,6-O-benzylidene-3-deoxy-3-[4-(3,4,5-trifluorophenyl)-1H-1,2,3-triazol-1-yl]-2-O-[(ditert-butyl-phosphonooxy)methyl]-1-thio-α-D-galactopyranoside

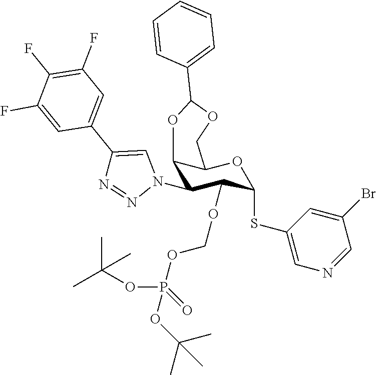

5-Bromopyridin-3-yl 4,6-O-benzylidene-3-deoxy-3-[4-(3,4,5-trifluorophenyl)-1H-1,2,3-triazol-1-yl]-1-thio-α-D-galactopyranoside (200 mg, 0.322 mmol), Silver oxide (149 mg, 0.644 mmol), NaI (96.5 mg, 0.644 mmol) and Molecular Sieves 4 Å (500 mg) were dissolved in DMF (6 mL) under a nitrogen atmosphere. Ditert-butyl chloromethyl phosphate (167 mg, 0.644 mmol) in DMF (0.5 mL) was added. The reaction was stirred over night at rt and LCMS showed all starting material consumed. The reaction was filtered through Celite® and solvent was removed. The residue was purified by Prep.-HPLC to give the title compound (260 mg, 0.308 mmol, yield: 95.8%) as white solid.

m/z calcd for [C₃₅H₃₉BrF₃N₄O₈PS]⁺ [M+H]⁺: 843.0, 845.0; found: 633.0, 635.0; 731.0, 733.0.

¹H NMR (400 MHz, DMSO) δ 8.73 (s, 1H), 8.70 (d, J=1.9 Hz, 1H), 8.68 (d, J=2.1 Hz, 1H), 8.30 (t, J=2.0 Hz, 1H), 7.72 (dd, J=8.9, 6.7 Hz, 2H), 7.34 (s, 5H), 6.40 (d, J=5.1 Hz, 1H), 5.62 (s, 1H), 5.30 (dd, J=11.6, 3.2 Hz, 1H), 5.19 (dd, J=9.8, 5.7 Hz, 1H), 5.13 (dd, J=9.8, 5.8 Hz, 1H), 5.04 (dd, J=11.5, 5.2 Hz, 1H), 4.65 (d, J=3.1 Hz, 1H), 4.42 (s, 1H), 4.14 (d, J=11.6 Hz, 1H), 4.01 (d, J=11.7 Hz, 1H), 1.31 (d, J=4.0 Hz, 18H).

### 5-Bromopyridin-3-yl 3-deoxy-3-[4-(3,4,5-trifluorophenyl)-1H-1,2,3-triazol-1-yl]-2-O-[(phosphonooxy)methyl]-1-thio-α-D-galactopyranoside

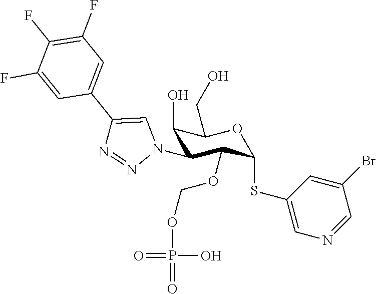

5-Bromopyridin-3-yl 4,6-O-benzylidene-3-deoxy-3-[4-(3,4,5-trifluorophenyl)-1H-1,2,3-triazol-1-yl]-2-O-[(ditert-butyl-phosphonooxy)methyl]-1-thio-α-D-galactopyranoside (210 mg, 0.249 mmol) was dissolved in 10% (v/v) TFA in DCM and the reaction mixture was stirred for 4 hours. LCMS indicated consumption of all starting material and that the product was the main peak. Solvent was removed and the residue was purified by Prep.-HPLC to give (73.6 mg, 0.114 mmol, yield: 45.9%) as a white solid. m/z calcd for [C20H19BrF3N4O8PS]⁻ [M−H]⁻: 641.0, 643.0; found: 641.0, 643.0. ¹H NMR (400 MHz, MeOD) δ 8.79-8.64 (m, 2H), 8.54 (d, J=2.0 Hz, 1H), 8.37 (s, 1H), 7.79-7.61 (m, 2H), 6.36 (d, J=5.1 Hz, 1H), 5.20 (dd, J=11.6, 5.2 Hz, 1H), 5.16-5.08 (m, 2H), 4.94-4.89 (m, 1H), 4.48 (t, J=5.9 Hz, 1H), 4.18 (s, 1H), 3.76-3.59 (m, 2H).

### Example 9

### 5-Bromopyridin-3-yl 3-[4-(4-chloro-3,5-difluorophenyl)-1H-1,2,3-triazol-1-yl]-3-deoxy-1-thio-α-D-galactopyranoside

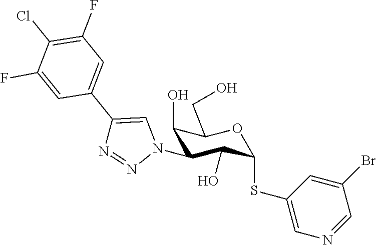

A solution of 5-bromopyridin-3-yl 2,4,6-tri-O-acetyl-3-azido-3-deoxy-1-thio-α-D-galactopyranoside (WO2016120403) (1.67 g, 3.32 mmol), K₂CO₃ (4.6 g, 33.2 mmol) and trimethyl-[2-(4-chloro-3,5-difluorophenyl)ethynyl]silane (1.58 g, 6.47 mmol) in MeOH (40 mL) and THF (40 mL) was stirred and degassed with nitrogen. Copper(II) sulfate pentahydrate (84 mg, 0.33 mmol) in hot water (10 mL) was added to (+)-sodium L-ascorbate (134 mg, 0.66 mmol) and the resulting suspension was added to the above mixture and stirred 18 h at 50° C. Silica was then added, and the suspension was concentrated, dried, and placed at the top of a column (silica). The column was eluted with EtOAc and concentrated. The residue was triturated in EtOAc/PE, the precipitate was isolated by filtration and gave the product (1.683 g, 92%). ESI-MS calcd for [C₁₉H₁₆BrClF₂N₄O₄S] [M+H]⁺: 549.0; found: 548.8. 1H NMR (400 MHz, Methanol-d₄) δ 8.69 (s, 1H), 8.60 (s, 1H), 8.57 (s, 1H), 8.32 (s, 1H), 7.66 (d, J=8.8 Hz, 2H), 5.92 (d, J=5.1 Hz, 1H), 5.03 (d, J=11.2 Hz, 1H), 4.95 (dd, J=11.3, 5.2 Hz, 1H), 4.49 (t, J=5.9 Hz, 1H), 4.21 (s, 1H), 3.78-3.65 (m, 2H).

### 5-Bromopyridin-3-yl 4,6-O-benzylidene-3-[4-(4-chloro-3,5-difluorophenyl)-1H-1,2,3-triazol-1-yl]-3-deoxy-1-thio-α-D-galactopyranoside

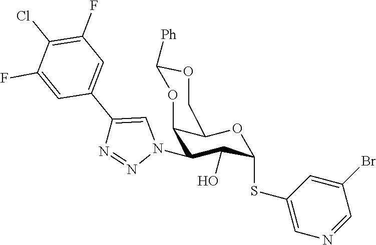

5-Bromopyridin-3-yl 3-[4-(4-chloro-3,5-difluorophenyl)-1H-1,2,3-triazol-1-yl]-3-deoxy-1-thio-α-D-galactopyranoside (1.625 g, 2.95 mmol) was stirred in MeCN (25 mL) and benzaldehyde dimethyl acetal (0.91 mL, 6.0 mmol) followed by p-toluenesulfonic acid monohydrate (100 mg, 0.53 mmol) were added and the mixture was stirred 17 h at 35° C. More benzaldehyde dimethyl acetal (0.91 mL, 6.0 mmol) and p-toluenesulfonic acid monohydrate (100 mg, 0.53 mmol) were added and the mixture was stirred 3 h at 40° C. Addition of Et₃N (0.30 mL) followed by water (30 mL), filtering and washing the precipitate with 33% aq MeOH and drying gave the product (1.647 g, 87%). ESI-MS nilz calcd for [C₂₆H₂₀BrClF₂N₄O₄] [M+H]⁺: 637.0; found: 637.0. ¹H NMR (400 MHz, DMSO-d₆) δ 8.92 (s, 1H), 8.68 (s, 1H), 8.63 (s, 1H), 8.27 (s, 1H), 7.80 (d, J=8.5 Hz, 2H), 7.39-7.28 (m, 5H), 6.25 (d, J=5.1 Hz, 1H), 6.18 (d, J=4.6 Hz, 1H), 5.57 (s, 1H), 5.13 (dd, J=11.3, 2.9 Hz, 1H), 4.91 (m, 1H), 4.57 (d, J=3.0 Hz, 1H), 4.29 (s, 1H), 4.12 (d, J=12.3 Hz, 1H), 3.92 (d, J=12.6 Hz, 1H).

### 5-Bromopyridin-3-yl 4,6-O-benzylidene-2-O-[(di-tert-butyl-phosphonooxy)methyl]-3-[4-(4-chloro-3,5-difluorophenyl)-1H-1,2,3-triazol-1-yl]-3-deoxy-1-thio-α-D-galactopyranoside

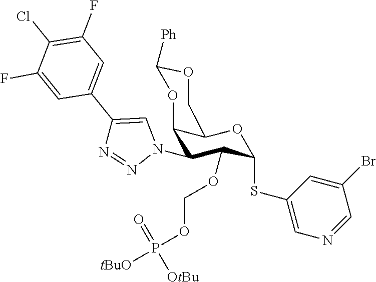

To a solution of 5-bromopyridin-3-yl 4,6-O-benzylidene-3-[4-(4-chloro-3,5-difluorophenyl)-1H-1,2,3-triazol-1-yl]-3-deoxy-1-thio-α-D-galactopyrano side (1.00 g, 1.489 mmol), KI (374 mg, 2.23 mmol) and di-tert-butyl chloromethyl phosphate (610 mg, 2.23 mmol) in DMF (10 mL) NaH (60% in oil, 90 mg, 2.23 mmol) was added. The mixture was stirred 2 h at rt, then partitioned between water and EtOAc. The organic phase was dried, evaporated and the residue was purified by chromatography (SiO₂, PE/EtOAc) to give the product (526 mg, 41%). ¹H NMR (400 MHz, DMSO-d₆) δ 8.79 (s, 1H), 8.69 (d, J=1.8 Hz, 1H), 8.67 (d, J=1.8 Hz, 1H), 8.29 (d, J=1.7 Hz, 1H), 7.72 (d, J=8.3 Hz, 2H), 7.34 (s, 5H), 6.40 (d, J=5.0 Hz, 1H), 5.62 (s, 1H), 5.31 (dd, J=11.5, 3.1 Hz, 1H), 5.19 (dd, J=9.8, 5.8 Hz, 1H), 5.13 (dd, J=9.9, 5.7 Hz, 1H), 5.05 (dd, J=11.5, 5.1 Hz, 1H), 4.66 (d, J=3.1 Hz, 1H), 4.42 (s, 1H), 4.15 (d, J=12.1 Hz, 1H), 4.01 (d, J=12.5 Hz, 1H), 1.31 (s, 9H), 1.30 (s, 9H).

### 5-Bromopyridin-3-yl 3-[4-(4-chloro-3,5-difluorophenyl)-1H-1,2,3-triazol-1-yl]-3-deoxy-2-O-[(phosphonooxy)methyl]-1-thio-α-D-galactopyranoside

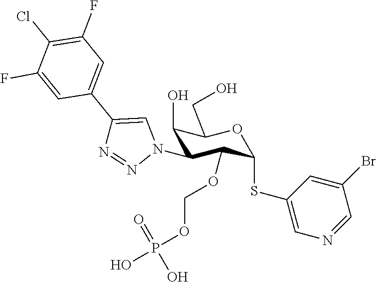

5-Bromopyridin-3-yl 4,6-O-benzylidene-2-O-[(di-tert-butyl-phosphonooxy)methyl]-3-[4-(4-chloro-3,5-difluorophenyl)-1H-1,2,3-triazol-1-yl]-3-deoxy-1-thio-α-D-galactopyranoside (515 mg, 0.59 mmol) was stirred 1.5 h at rt in 80% aq TFA (5.0 mL). The mixture was poured onto ice/water, the precipitate was collected by filtration and dissolved in EtOAc. The solution was washed with water and the organic phase was collected and evaporated. The residue was triturated in diethyl ether to afford crude title compound (255 mg, 58%). Part of this material (70 mg) was purified by prep-HPLC (20-50% ACN/20 min) to give the title compound (50 mg, 81%). ESI-MS calcd for [C₂₀H₁₉BrClF₂N₄O₈PS] [M+H]⁺: 659.0; found: 658.8.

¹H NMR (400 MHz, Methanol-d₄) δ 8.72 (s, 1H), 8.63 (s, 1H), 8.60 (s, 1H), 8.39 (t, J=1.9 Hz, 1H), 7.66 (d, J=8.0 Hz, 2H), 6.21 (d, J=4.8 Hz, 1H), 5.22 (dd, J=9.8, 5.9 Hz, 1H), 5.17 (dd, J=11.5, 2.5 Hz, 1H), 5.12 (dd, J=11.5, 4.8 Hz, 1H), 5.06 (dd, J=11.1, 5.9 Hz, 1H), 4.53 (t, J=6.0 Hz, 1H), 4.22 (m, 1H), 3.76-3.66 (m, 2H).

### REFERENCES

Aits S, Kricker J, Liu B, Ellegaard A M, Hamalisto S, Tvingsholm S, Corcelle-Termeau E, Ht gh S, Farkas T, Holm Jonassen A, Gromova I, Mortensen M, Jäättelä M. (2015) Sensitive detection of lysosomal membrane permeabilization by lysosomal galectin puncta assay Autophagy. 2015; 11(8):1408-24.
Almkvist, J., Fäldt, J., Dahlgren, C., Leffler, H., and Karlsson, A. (2001) Lipopolysaccharide-induced gelatinase granule mobilization primes neutrophils for activation by galectin-3 and f-Met-Leu-Phe. Infect. Immun. Vol. 69: 832-837.
Arthur C M, Baruffi M D, Cummings R D, Stowell S R. (2015) Evolving mechanistic insights into galectin functions. Methods Mol Biol. 1207:1-35.
Blanchard H, Yu X, Collins P M, Bum-Erdene K. (2014) Galectin-3 inhibitors: a patent review (2008-present). Expert Opin Ther Pat. 2014 October; 24(10):1053-65.
Blidner A G, Mendez-Huergo S P, Cagnoni A J, Rabinovich G A. (2015) Re-wiring regulatory cell networks in immunity by galectin-glycan interactions. FEBS Lett. 2015 Sep. 6. pii: S0014-5793(15)00807-8.
Chen, W.-S., Leffler H., Nilsson, U. J., Panjwani, N. (2012). Targeting Galectin-1 and Galectin-3 Attenuates VEGF-A-induced Angiogenesis; Mol. Biol. Cell (suppl), Abstract No. 2695.
Clare D K, Magescas J, Piolot T, Dumoux M, Vesque C, Pichard E, Dang T, Duvauchelle B, Poirier F, Delacour D. (2014) Basal foot MTOC organizes pillar MTs required for coordination of beating cilia. Nat Commun. 5:4888.
Cumpstey, I., Carlsson, S., Leffler, H. and Nilsson, U. J. (2005) Synthesis of a phenyl thio-β-D-galactopyranoside library from 1,5-difluoro-2,4-dinitrobenzene: discovery of efficient and selective monosaccharide inhibitors of galectin-7. Org. Biomol. Chem. 3: 1922-1932.
Cumpstey, I., Sundin, A., Leffler, H. and Nilsson, U. J. (2005) C₂-Symmetrical thiodigalactoside bis-benzamido derivatives as high-affinity inhibitors of galectin-3: Efficient lectin inhibition through double arginine-arene interactions. Angew. Chem. Int. Ed. 44: 5110-5112.
Cumpstey, I., Salomonsson, E., Sundin, A., Leffler, H. and Nilsson, U. J. (2008) Double affinity amplification of galectin-ligand interactions through arginine-arene interactions: Synthetic, thermodynamic, and computational studies with aromatic diamido-thiodigalactosides. Chem. Eur. J. 14: 4233-4245.
Delaine, T., Cumpstey, I., Ingrassia, L., Le Mercier, M., Okechukwu, P., Leffler, H., Kiss, R., and Nilsson, U. J. (2008). Galectin-Inhibitory Thiodigalactoside Ester Derivatives Have Anti-Migratory Effects in Cultured Lung and Prostate Cancer Cells. J Med Chem 51; 8109-8114.
Demotte, N., Wieers, G., van der Smissen, P., Moser, M., Schmidt, C., Thielemans, K., et al., (2010). Cancer Res. 70; 7476-7488.
Ebrahim A H, Alalawi Z, Mirandola L, Rakhshanda R, Dahlbeck S, Nguyen D, Jenkins M I, Grizzi F, Cobos E, Figueroa J A, Chiriva-Internati M (2014)Galectins in cancer: carcinogenesis, diagnosis and therapy. Ann Transl Med. 2014 September; 2(9):88.
Elola M T, Blidner A G, Ferragut F, Bracalente C, Rabinovich G A. (2015) Assembly, organization and regulation of cell-surface receptors by lectin-glycan complexes. Biochem J. 2015 Jul. 1; 469(1):1-16.
Farkas, I.; Szabo, I. F.; BognAr, R.; Anderle, D. Carbohydr. Res. 1976, 48, 136-138.
Funasaka T, Raz A, Nangia-Makker P. (2014) Nuclear transport of galectin-3 and its therapeutic implications. Semin Cancer Biol. 2014 August; 27:30-8.
Giguére, D.; Bonin, M.-A.; Cloutier, P.; Patnam, R.; St-Pierre, C.; Sato, S.; Roy, R. Bioorganic & Medicinal Chemistry 2008, 16, 7811-7823.
Giguére, D.; André, S.; Bonin, M.-A.; Bellefleur, M.-A.; Provencal, A.; Cloutier, P.; Pucci, B.; Roy, R.; Gabius, H.-J. Bioorganic & Medicinal Chemistry 2011, 19, 3280-3287
Giguere, D., Patnam, R., Bellefleur, M.-A., St.-Pierre, C., Sato, S., and Roy, R. (2006). Carbohydrate triazoles and isoxazoles as inhibitors of galectins-1 and -3. Chem Commun: 2379-2381.
Glinsky, G. V., Price, J. E., Glinsky, V. V., Mossine, V. V., Kiriakova, G., and Metcalf, J. B. (1996). Cancer Res 56: 5319-5324.
Synthetic Galectin-3 Inhibitor Increases Metastatic Cancer Cell Sensitivity to Taxol-Induced Apoptosis In Vitro and In Vivo. Neoplasia 11; 901-909.
Huflejt, M. E. and Leffler, H. (2004) Galectin-4 in normal tissues and cancer. Glycoconj. J. 20: 247-255.
Ingrassia et al. (2006) A Lactosylated Steroid Contributes in Vivo Therapeutic Benefits in Experimental Models of Mouse Lymphoma and Human Glioblastoma. J. Med. CHem. 49: 1800-1807.
John, C. M., Leffler, H., Kahl-Knutsson, B., Svensson, I., and Jarvis, G. A. (2003) Truncated Galectin-3 Inhibits Tumor Growth and Metastasis in Orthotopic Nude Mouse Model of Human Breast Cancer. Clin. Cancer Res. 9: 2374-2383.
Kouo, T., Huang, L., Pucsek, A. B., Cao, M., Solt, S., Armstrong, T., Jaffee, E. (2015) Cancer Immonol. Res. 3: 412-23
Leffler, H. and Barondes, S. H. (1986) Specificity of binding of three soluble rat lung lectins to substituted and unsubstituted mammalian beta-galactosides. J. Biol. Chem. 261:10119-10126.
Leffler, H. Galectins Structure and Function—A Synopsis in Mammalian Carbohydrate Recognition Systems (Crocker, P. ed.) Springer Verlag, Heidelberg, 2001 pp. 57-83.
Leffler, H., Carlsson, S., Hedlund, M., Qian, Y. and Poirier, F. (2004) Introduction to galectins. Glycoconj. J. 19: 433-440.
Lepur A, Salomonsson E, Nilsson U J, Leffler H. (2012) Ligand induced galectin-3 protein self-association. J Biol Chem. 2012 Jun. 22; 287(26):21751-6.
Li L C, Li J, Gao J. (2014) Functions of galectin-3 and its role in fibrotic diseases. J Pharmacol Exp Ther. 2014 November; 351(2):336-43.
MacKinnon, A. C., Farnworth, S. L., Henderson, N. C., Hodkinson, P. S., Kipari, T., Leffler, H., Nilsson, U. J., Haslett, C., Hughes, J., and Sethi T. (2008). Regulation of alternative macrophage activation by Galectin-3. J. Immun. 180; 2650-2658.
Mackinnon, A., Gibbons, M., Farnworth, S., Leffler, H., Nilsson, U. J., Delaine, T., Simpson, A., Forbes, S., Hirani, N., Gauldie, J., and Sethi T. (2012). Regulation of TGF-β1 driven lung fibrosis by Galectin-3. Am. J. Resp. Crit. Care Med., in press.
Massa, S. M., Cooper, D. N. W., Leffler, H., Barondes, S. H. (1993) L-29, an endogenous lectin, binds to glycoconjugate ligands with positive cooperativity. Biochemistry 32: 260-267.
Melero, I., Berman, D. M., Aznar, M. A., Korman, A. J., Gracia, J. L. P., Haanen, J. (2015) Nature Reviews Cancer, 15: 457-472
Partridge, E. A., Le Roy, C., Di Guglielmo, G. M., Pawling, J., Cheung, P., Granovsky, M., Nabi, I. R., Wrana, J. L., and Dennis, J. W. (2004). Regulation of cytokine receptors by Golgi N-glycan processing and endocytosis. Science 306: 120-124.
Pienta, Naik, H., Akhtar, A., Yamazaki, K., Reploge, T. S., Lehr, J., Donat, T. L., Tait, L., Hogan, V., and Raz, A. (1995). Inhibition of spontaneous metastasis in a rat prostate cancer model by oral administration of modified citrus pectin. J Natl Cancer Inst 87, 348-353.
Ramos-Soriano, J.; Niss, U.; Angulo, J.; Angulo, M.; Moreno-Vargas, A. J.; Carmona, A. T.; Ohlson, S.; Robina, I. Chem. Eur. J. 2013, 19, 17989-18003.
Ruvolo, P. P. Biochim. Biophys Acta. Molecular cell research (2015) E-pub ahead of print, title: Galectin-3 as a guardian of the tumor microenvironment, published on-line 8 Apr. 2015: (http://www.sciencedirect.com/science/article/pii/S0167488915002700),
Salameh, B. A., Leffler, H. and Nilsson, U. J. (2005) Bioorg. Med. Chem. Lett. 15: 3344-3346.
Salameh, B. A., Cumpstey, I., Sundin, A., Leffler, H., and Nilsson, U. J. (2010). 1H-1,2,3-Triazol-1-yl thiodigalactoside derivatives as high affinity galectin-3 inhibitors. Bioorg Med Chem 18: 5367-5378.
Salomonsson, E., Larumbe, A., Tejler, J., Tullberg, E., Rydberg, H., Sundin, A., Khabut, A., Frejd, T., Lobsanov, Y. D., Rini, J. M., Nilsson, U. J., and Leffler, H (2010). Monovalent interactions of galectin-1. Biochemistry 49: 9518-9532.
Sörme, P., Qian, Y., Nyholm, P.-G., Leffler, H., Nilsson, U. J. (2002) Low micromolar inhibitors of galectin-3 based on 3′-derivatization of N-acetyllactosamine ChemBioChem 3:183-189.
Sörme, P., Kahl-Knutsson, B., Wellmar, U., Nilsson, U. J., and Leffler H. (2003a) Fluorescence polarization to study galectin-ligand interactions. Meth. Enzymol. 362: 504-512.

Sörme, P., Kahl-Knutsson, B., Wellmar, U., Magnusson, B.-G., Leffler H., and Nilsson, U. J. (2003b) Design and synthesis of galectin inhibitors. Meth. Enzymol. 363: 157-169.
Sörme, P., Kahl-Knutsson, B., Huflejt, M., Nilsson, U. J., and Leffler H. (2004) Fluorescence polarization as an analytical tool to evaluate galectin-ligand interactions. Anal. Biochem. 334: 36-47.
Thijssen V L, Heusschen R, Caers J, Griffioen A W. (2015) Galectin expression in cancer diagnosis and prognosis: A systematic review. Biochim Biophys Acta. 2015 April; 1855(2):235-47.
Toscano, M. A., Bianco, G. A., Ilarregui, J. M., Croci, D. O., Correale, J., Hernandez, J. D., Zwirner, N. W., Poirier, F., Riley, E. M., Baum, L. G., et al. (2007). Differential glycosylation of TH1, TH2 and TH-17 effector cells selectively regulates susceptibility to cell death. Nat Immunol 8: 825-834.
Viguier M, Advedissian T, Delacour D, Poirier F, Deshayes F. (2014) Galectins in epithelial functions. Tissue Barriers. 2014 May 6; 2:e29103.

1-25. (canceled)

26. A prodrug compound of formula (I) 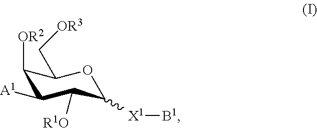
wherein the pyranose ring is α- or β-D-galactopyranose (as indicated by wavy line);
wherein:
A¹ is selected from the group consisting of i) an aryl; ii) an aryl substituted with at least one from the group consisting of a halogen; CN; C₂₋₆ alkenyl; C₂₋₆ alkynyl; carboxyl; C₁₋₆ alkoxy; C₁₋₆ thioalkyl; C₁₋₆ alkyl; nitro; thio; C₁₋₆ alkylthio; amino; hydroxy; C₁₋₆ carbonyl; an amino; and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; iii) a C₁₋₆ alkoxy; iv) a C₁₋₆ alkoxy substituted with at least one from the group consisting of a halogen; a C₁₋₆ alkyl; a heteroaryl; a heteroaryl substituted with at least one from the group consisting of halogen, CN, C₂₋₆ alkenyl, C₂₋₆ alkynyl, carboxyl, C₁₋₆ alkoxy, C₁₋₆ thioalkyl, amino, C₁₋₆ alkyl, C₁₋₆ alkyl substituted with at least one halogen, C₁₋₆ alkoxy, C₁₋₆ alkoxy substituted with at least one halogen, a five or six membered heteroaromatic ring, a five or six membered heteroaromatic ring substituted with at least one from the group consisting of halogen, CN, C₂₋₆ alkenyl, C₂₋₆ alkynyl, carboxyl, C₁₋₆ alkoxy, C₁₋₆ thioalkyl, C₁₋₆ alkyl, C₁₋₆ alkyl substituted with at least one halogen, C₁₋₆ alkoxy, and C₁₋₆ alkoxy substituted with at least one halogen, an aryl, and an aryl substituted with at least one from the group consisting of halogen, CN, C₂₋₆ alkenyl, C₂₋₆ alkynyl, carboxyl, C₁₋₆ alkoxy, C₁₋₆ thioalkyl, C₁₋₆ alkyl, C₁₋₆ alkyl substituted with at least one halogen, C₁₋₆ alkoxy, and C₁₋₆ alkoxy substituted with at least one halogen; an amino; and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; v) a C₁₋₆ alkylamino; vi) a C₁₋₆ alkylamino substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; vii) a heteroaryl; viii) a heteroaryl substituted with at least one from the group consisting of a halogen; CN; C₂₋₆ alkenyl; C₂₋₆ alkynyl; carboxyl; C₁₋₆ alkoxy; C₁₋₆ thioalkyl; an amino; an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; an aryl; an aryl substituted with at least one from the group consisting of a halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a heteroaryl; a heteroaryl substituted with at least one from the group consisting of halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₃₋₇ cycloalkyl, C₃₋₇ cycloalkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₃₋₇ cycloalkoxy, and C₃₋₇ cycloalkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a C₁₋₆ carbonyl; a C₁₋₆ carbonyl substituted with at least one from the group consisting of halogen, C₁₋₆ alkoxy, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, and an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; ix) a heterocycle; x) a heterocycle substituted with at least one from the group consisting of halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₃₋₇ cycloalkyl, C₃₋₇ cycloalkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₃₋₇ cycloalkoxy, and C₃₋₇ cycloalkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a C₁₋₆ carbonyl; a C₁₋₆ carbonyl substituted with at least one from the group consisting of halogen, C₁₋₆ alkoxy, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, and an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; xi) a C₁₋₆ alkyl; xii) a C₁₋₆ alkyl substituted with at least one from the group consisting of halogen; C₁₋₆ alkoxy; C₁₋₆ alkyl; C₃₋₇ cycloalkyl; nitro; thio; C₁₋₆ alkylthio; amino; hydroxy; and C₁₋₆ carbonyl; xiii) a C₁₋₆ carbonyl; xiv) a C₁₋₆ carbonyl substituted with at least one from the group consisting of a C₁₋₆ alkyl; a C₂₋₆ alkenyl; an aryl; a heteroaryl; and a heterocycle; xv) a C₁₋₆ alkyl-CONH—; xvi) a C₁₋₆ alkyl-CONH— substituted on one or more alkyl carbon with at least one from the group consisting of a heteroaryl; a heteroaryl substituted with at least one from the group consisting of a halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; nitro, thio, C₁₋₆ alkylthio, amino, hydroxy, C₁₋₆ carbonyl, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; an aryl; and an aryl substituted with at least one from the group consisting of a halogen, CN, C₂₋₆ alkenyl, C₂₋₆ alkynyl, carboxyl, C₁₋₆ alkoxy, C₁₋₆ thioalkyl, C₁₋₆ alkoxy substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; or an in vivo metabolizable group of A¹;
X¹ is selected from the group consisting of O, S, SO, SO₂, C═O, amino, amino substituted with a C₁₋₆ alkyl, and CR′R″ wherein R′ and R″ are independently selected from hydrogen, OH, or halogen; or an in vivo metabolizable group of X¹;
B¹ is selected from the group consisting of a) a C₁₋₆ alkyl, b) a C₁₋₆ alkyl substituted with at least one from the group consisting of a five or six membered heteroaromatic ring; a five or six membered heteroaromatic ring substituted with at least one from the group consisting of cyano, halogen, C₁₋₆ alkyl, C₁₋₆ alkyl substituted with at least one from the group consisting of halogen, hydroxy and C₁₋₆ alkyl, C₁₋₆ alkoxy, C₁₋₆ alkoxy substituted with at least one from the group consisting of halogen, hydroxy and C₁₋₆ alkyl, hydroxy, and R^{#}—CONH— wherein R^{#} is selected from the group consisting C₁₋₆ alkyl and C₁₋₆ cycloalkyl; an aryl; and an aryl substituted with at lest one from the group consisting of cyano, halogen, C₁₋₆ alkyl, C₁₋₆ alkyl substituted with at least one from the group consisting of halogen, hydroxy and C₁₋₆ alkyl, C₁₋₆ alkoxy, C₁₋₆ alkoxy substituted with at least one from the group consisting of halogen, hydroxy and C₁₋₆ alkyl, hydroxy, and R^{#}—CONH— wherein R^{#} is selected from the group consisting C₁₋₆ alkyl and C₁₋₆ cycloalkyl; c) an aryl; d) an aryl substituted with at least one from the group consisting of halogen; cyano; hydroxy; carboxyl; carboxamid; carboxamid substituted with at least one from the group consisting of C₁₋₆ alkyl and C₃₋₆ cycloalkyl; C₁₋₆ alkyl; C₁₋₆ alkyl substituted with at least one from the group consisting of halogen, hydroxy, and R^{&}—CONH— wherein R^{&} is selected from the group consisting C₁₋₆ alkyl and C₁₋₆ cycloalkyl; C₁₋₆ cycloalkyl; C₁₋₆ cycloalkyl substituted with at least one from the group consisting of halogen, hydroxy, and R^{%}—CONH— wherein R^{%} is selected from the group consisting C₁₋₆ alkyl and C₁₋₆ cycloalkyl; C₁₋₆ alkoxy; C₁₋₆ alkoxy substituted with at least one from the group consisting of halogen, hydroxy, and R^{§} —CONH— wherein R^{§§} is selected from the group consisting C₁₋₆ alkyl and C₁₋₆ cycloalkyl; C₃₋₆ cycloalkoxy; C₃₋₆ cycloalkoxy substituted with at least one from the group consisting of halogen, hydroxy, and R*—CONH— wherein R* is selected from the group consisting C₁₋₆ alkyl and C₁₋₆ cycloalkyl; amino; amino substituted with at least one from the group consisting of C₁₋₆ alkyl and C₁₋₆ cycloalkyl; and R**—CONH— wherein R** is selected from the group consisting C₁₋₆ alkyl and C₁₋₆ cycloalkyl; e) a C₄₋₁₀ cycloalkyl, f) a C₄₋₁₀ cycloalkyl substituted with at least one from the group consisting of cyano, halogen, C₁₋₆ alkyl, C₁₋₆ alkyl substituted with at least one from the group consisting of halogen, hydroxy and C₁₋₆ alkyl, C₁₋₆ alkoxy, C₁₋₆ alkoxy substituted with at least one from the group consisting of halogen, hydroxy and C₁₋₆ alkyl, hydroxy, and R^{##}—CONH— wherein R^{##} is selected from the group consisting C₁₋₆ alkyl and C₁₋₆ cycloalkyl; and g) a heterocycle substituted with at least one from the group consisting of halogen; cyano; hydroxy; carboxyl; carboxamid; carboxamid substituted with at least one from the group consisting of C₁₋₆ alkyl and C₃₋₆ cycloalkyl; C₁₋₆ alkyl; C₁₋₆ alkyl substituted with at least one from the group consisting of halogen, hydroxy, and R^{&&}—CONH— wherein R^{&&} is selected from the group consisting C₁₋₆ alkyl and C₁₋₆ cycloalkyl; C₁₋₆ cycloalkyl; C₁₋₆ cycloalkyl substituted with at least one from the group consisting of halogen, hydroxy, and R^{%%}—CONH— wherein R^{%%} is selected from the group consisting C₁₋₆ alkyl and C₁₋₆ cycloalkyl; C₁₋₆ alkoxy; C₁₋₆ alkoxy substituted with at least one from the group consisting of halogen, hydroxy, and R^{§§} —CONH— wherein R^{§§} is selected from the group consisting C₁₋₆ alkyl and C₁₋₆ cycloalkyl; C₃₋₆ cycloalkoxy; C₃₋₆ cycloalkoxy substituted with at least one from the group consisting of halogen, hydroxy, and R^{a}—CONH— wherein R^{a} is selected from the group consisting C₁₋₆ alkyl and C₁₋₆ cycloalkyl; amino; amino substituted with at least one from the group consisting of C₁₋₆ alkyl and C₁₋₆ cycloalkyl; R^{aa}—CONH— wherein R^{aa} is selected from the group consisting C₁₋₆ alkyl and C₁₋₆ cycloalkyl; a heteroaryl substituted with at least one from the group consisting of a halogen; an amino; an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; an aryl; an aryl substituted with at least one from the group consisting of a halogen, cyano, C₁₋₆ alkoxy, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a heteroaryl; a heteroaryl substituted with at least one from the group consisting of halogen, cyano, C₁₋₆ alkoxy, C₁₋₆ alkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₁₋₆ alkyl, C₁₋₆ alkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, nitro, thio, C₁₋₆ alkylthio, amino, hydroxy, C₁₋₆ carbonyl, an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, C₃₋₇ cycloalkyl, C₃₋₇ cycloalkyl substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl, C₃₋₇ cycloalkoxy, and C₃₋₇ cycloalkoxy substituted with at least one from the group consisting of a halogen, an amino, and an amino substituted with at least one from the group consisting of halogen, C₁₋₆ alkoxy, C₁₋₆ alkyl, nitro, thio, C₁₋₆ alkylthio, amino, hydroxy and C₁₋₆ carbonyl; a C₁₋₆ carbonyl; and a C₁₋₆ carbonyl substituted with at least one from the group consisting of halogen, C₁₋₆ alkoxy, C₁₋₆ alkyl, C₁₋₆ alkyl substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl, nitro, thio, C₁₋₆ alkylthio, amino, and an amino substituted with at least one from the group consisting of halogen, C₁₋₆ alkoxy, C₁₋₆ alkyl, C₃₋₇ cycloalkyl, nitro, thio, C₁₋₆ alkylthio, amino, hydroxy and C₁₋₆ carbonyl; or an in vivo metabolizable group of B¹;
R¹ is selected from the group consisting of hydrogen and an in vivo metabolizable group;
R² is selected from the group consisting of hydrogen and an in vivo metabolizable group; and
R³ is selected from the group consisting of hydrogen and an in vivo metabolizable group;
or a pharmaceutically acceptable salt or solvate thereof;
with the proviso that at least one in vivo metabolizable group is present in at least one from the group consisting of A¹, X¹, B¹, R¹, R² and R³.

27. The prodrug compound of claim 26, wherein the prodrug is bioactivated outside a mammalian cell.

28. The prodrug compound of claim 26, wherein the prodrug is bioactivated inside a mammalian cell.

29. The prodrug compound of claim 26, wherein the in vivo metabolizable group is selected from the group consisting of carbamate, ether, phosphate, sulphate, oxy alkyl phosphate, oxy alkyl sulphate, N-Mannich base, carbonate, amide, ester, N-acylsulphonamides, sulfonamides, imines, acyloxyalkylamines, phosphates, phosphoroimidates, azoconjugates, carbonyloxymethyl, acetylthioethanol, dithioethanol, cyclosal, Hep-direct, phosphorodiimidates, ProTide phosphoroimidates, Pro Tide phosphonoimidates, alkoxyalkylmonoeters and acetyl.

30. The prodrug compound of claim 26 having formula II 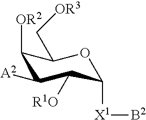
wherein the pyranose ring is α-D-galactopyranose,
wherein:
A² is selected from 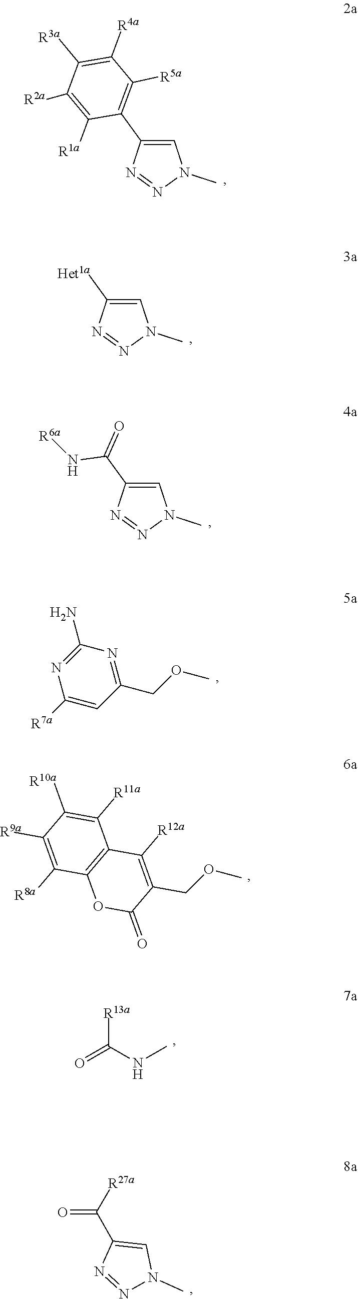
wherein Het^{1a} is selected from a five or six membered heteroaromatic ring, optionally substituted with a group selected from Br; F; Cl; CN; NR^{19a}R^{20a}, wherein R^{19a} and R^{29a} are independently selected from H, C₁₋₃ alkyl, cyclopropyl, iso-propyl, —C(═O)—R^{21a}, wherein R^{21a} is selected from H and C₁₋₃ alkyl; C₁₋₃ alkyl, optionally substituted with a F; cyclopropyl, optionally substituted with a F; iso-propyl, optionally substituted with a F; O-cyclopropyl optionally substituted with a F; O-isopropyl optionally substituted with a F; and OC₁₋₃ alkyl optionally substituted with a F;
wherein R^{1a}-R^{5a} are independently selected from H, CN, NH₂, F, methyl optionally substituted with a F, and OCH₃ optionally substituted with a F;
wherein R^{6a} is selected from C₁₋₆ alkyl optionally substituted with a halogen, branched C₃₋₆ alkyl and C₃₋₇ cycloalkyl;
wherein R^{7a} is selected from a five or six membered heteroaromatic ring, optionally substituted with a group selected from Br, F, Cl, methyl optionally substituted with a F, and OCH₃ optionally substituted with a F, and a phenyl optionally substituted with a group selected from Br, F, Cl, methyl optionally substituted with a F, and OCH₃ optionally substituted with a F;
wherein R^{8a}-R^{12a} are independently selected from H, F, methyl optionally substituted with a F, and OCH₃ optionally substituted with a F;
wherein R^{13a} is a five or six membered heteroaromatic ring optionally substituted with a group selected from H, OH, F, methyl optionally substituted with a F, and OCH₃ optionally substituted with a F, or an aryl, such as phenyl or naphthyl, optionally substituted with a group selected from H, OH, F, methyl optionally substituted with a F, and OCH₃ optionally substituted with a F;
X¹ is selected from S, SO, SO₂, O, C═O, and CR^{32a}R^{33a} wherein R^{32a} and R^{33a} are independently selected from hydrogen, OH, or halogen;
wherein R^{27a} is selected from a C₁₋₆ alkyl, branched C₃₋₆ alkyl, C₁₋₆ alkoxy and branched C₃₋₆ alkoxy;
B² is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{14a}—CONH— wherein R^{14a} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{15a}—CONH— wherein R^{15a} is selected from C₁₋₃ alkyl and cyclopropyl; b) an aryl, such as phenyl or naphthyl, optionally substituted with a group selected from a halogen; CN; —COOH; —CONR^{22a}R^{23a}, wherein R^{22a} and R^{23a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28a}R^{29a}, wherein R^{28a} and R^{29a} are independently selected from H, C₁₋₃ alkyl and isopropyl; OH; and R^{16a}—CONH— wherein R^{16a} is selected from C₁₋₃ alkyl and cyclopropyl; c) a C₅₋₇ cycloalkyl, optionally substituted with a substituent selected from a halogen, CN, methyl optionally substituted with a F, OCH₃ optionally substituted with a F, OCH₂CH₃ optionally substituted with a F, OH, and R^{17a}—CONH— wherein R^{17a} is selected from C₁₋₃ alkyl and cyclopropyl; and d) a heterocycle, such as heteroaryl or heterocycloalkyl, optionally substituted with a group selected from a halogen; CN; —COOH; —CONR^{24a}R^{25a}, wherein R^{24a} and R^{25a} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30a}R^{31a}, wherein R^{30a} and R^{31a} are independently selected from H, C₁₋₃ alkyl and isopropyl; OH; and R^{18a}—CONH— wherein R^{18a} is selected from C₁₋₃ alkyl and cyclopropyl; e) a C₁₋₆ alkyl or branched C₃₋₆ alkyl;
R¹ is selected from the group consisting of hydrogen and an in vivo metabolizable group;
R² is selected from the group consisting of hydrogen and an in vivo metabolizable group; and
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³.

31. The prodrug compound of claim 26 having formula II 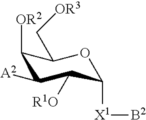
wherein the pyranose ring is α-D-galactopyranose,
A² is selected from 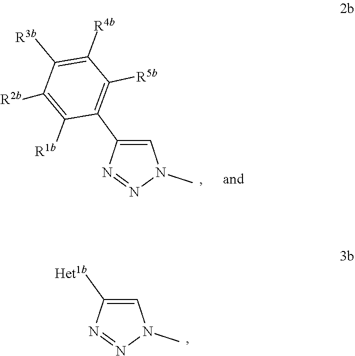
wherein Het^{1b} is selected from a pyridinyl, optionally substituted with a group selected from H, CN, Br, Cl, I, F, methyl optionally substituted with a F, OCH₃ optionally substituted with a F, and SCH₃ optionally substituted with a F; or a pyrimidyl, optionally substituted with a group selected from H, CN, Br, Cl, I, F, methyl optionally substituted with a F, OCH₃ optionally substituted with a F, and SCH₃ optionally substituted with a F;
wherein R^{1b}-R^{5b} are independently selected from a group consisting of H, CN, Br, Cl, I, F, methyl optionally substituted with a F, OCH₃ optionally substituted with a F, and SCH₃ optionally substituted with a F;
X¹ is selected from S, SO, and SO₂;
B² is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{14b}—CONH— wherein R^{14b} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{15b}—CONH— wherein R^{15b} is selected from C₁₋₃ alkyl and cyclopropyl; b) an aryl, such as phenyl or naphthyl, optionally substituted with a group selected from a halogen; CN; —COOH; —CONR^{22b}R^{23b}, wherein R^{22b} and R^{23b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28b}R^{29b}, wherein R^{28b} and R^{29b} are independently selected from H, C₁₋₃ alkyl and isopropyl; OH; and R^{16b}—CONH— wherein R^{16b} is selected from C₁₋₃ alkyl and cyclopropyl; c) a C₅₋₇ cycloalkyl, optionally substituted with a substituent selected from a halogen, CN, methyl optionally substituted with a F, OCH₃ optionally substituted with a F, OCH₂CH₃ optionally substituted with a F, OH, and R^{17b}—CONH— wherein R^{17b} is selected from C₁₋₃ alkyl and cyclopropyl; and d) a heterocycle, such as heteroaryl or heterocycloalkyl, optionally substituted with a group selected from a halogen; CN; —COOH; —CONR^{24b}R^{25b}, wherein R^{24b} and R^{25b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30b}R^{31b}, wherein R^{30b} and R^{31b} are independently selected from H, C₁₋₃ alkyl and isopropyl; OH; and R^{18b}—CONH— wherein R^{18b} is selected from C₁₋₃ alkyl and cyclopropyl; e) a C₁₋₆ alkyl or branched C₃₋₆ alkyl;
R¹ is selected from the group consisting of hydrogen and an in vivo metabolizable group;
R² is selected from the group consisting of hydrogen and an in vivo metabolizable group; and
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³.

32. The prodrug compound of claim 31,
wherein:
A² is 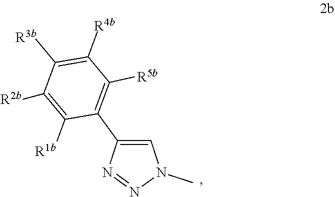
wherein
R^{1b}-R^{5b} are independently selected from a group consisting of H, CN, Br, Cl, I, F, methyl optionally substituted with a F, OCH₃ optionally substituted with a F, and SCH₃ optionally substituted with a F;
X¹ is S;
B² is selected from b) a phenyl, optionally substituted with a group selected from a halogen; CN; —COOH; —CONR^{22b}R^{23b}, wherein R^{22b} and R^{23b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{28b}R^{29b}, wherein R^{28b} and R^{29b} are independently selected from H, C₁₋₃ alkyl and isopropyl; OH; and R^{16b}—CONH— wherein R^{16b} is selected from C₁₋₃ alkyl and cyclopropyl; d) a heteroaryl, optionally substituted with a group selected from a halogen; CN; —COOH; —CONR^{24b}R^{25b}, wherein R^{24b} and R^{25b} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{30b}R^{31b}, wherein R^{30b} and R^{31b} are independently selected from H, C₁₋₃ alkyl and isopropyl; OH; and R^{18b}—CONH— wherein R^{18b} is selected from C₁₋₃ alkyl and cyclopropyl;
R¹ is selected from the group consisting of hydrogen and an in vivo metabolizable group;
R² is selected from the group consisting of hydrogen and an in vivo metabolizable group; and
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³.

33. The prodrug compound of claim 32,
wherein:
A² is 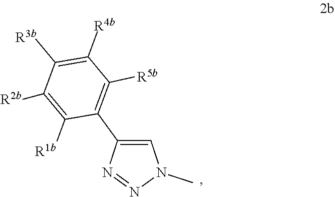
wherein
R^{1b}-R^{5b} are independently selected from a group consisting of H, Cl and F;
X¹ is S;
B² is selected from b) a phenyl substituted with a halogen; and d) a heteroaryl substituted with a cyano, a halogen, or a cyano and a halogen;
R¹ is selected from the group consisting of hydrogen and an in vivo metabolizable group;
R² is selected from the group consisting of hydrogen and an in vivo metabolizable group; and
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³.

34. The prodrug compound of claim 32,
wherein A² is 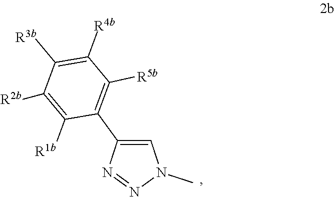
wherein
R^{1b}-R^{5b} are independently selected from a group consisting of H and F;
X¹ is S;
B² is selected from b) a phenyl substituted with a halogen; and d) a heteroaryl substituted with a halogen;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³.

35. The prodrug compound of claim 34,
wherein A² is 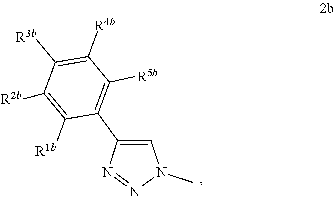
wherein
R^{1b} and R^{5b} are hydrogen, and at least one of R^{2b}-R^{4b} is F;
X¹ is S;
B² is selected from b) a phenyl substituted with a Cl; and d) a pyridinyl substituted with a Br;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³.

36. The prodrug compound of claim 33,
wherein A² is 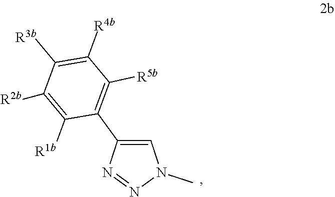
wherein
R^{1b} and R^{5b} are hydrogen, and R^{2b}-R^{4b} is selected from the group consisting of Cl and F;
X¹ is S;
B² is selected from d) a pyridinyl substituted with a group selected from Cl, Br and CN;
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³.

37. The prodrug compound of claim 26 having formula II 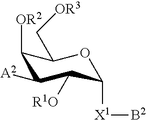
wherein the pyranose ring is α-D-galactopyranose,
A2 is 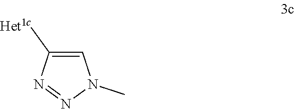
wherein Het^{1c} is a five or six membered heteroaromatic ring selected from the group consisting of formulas 2 to 9: 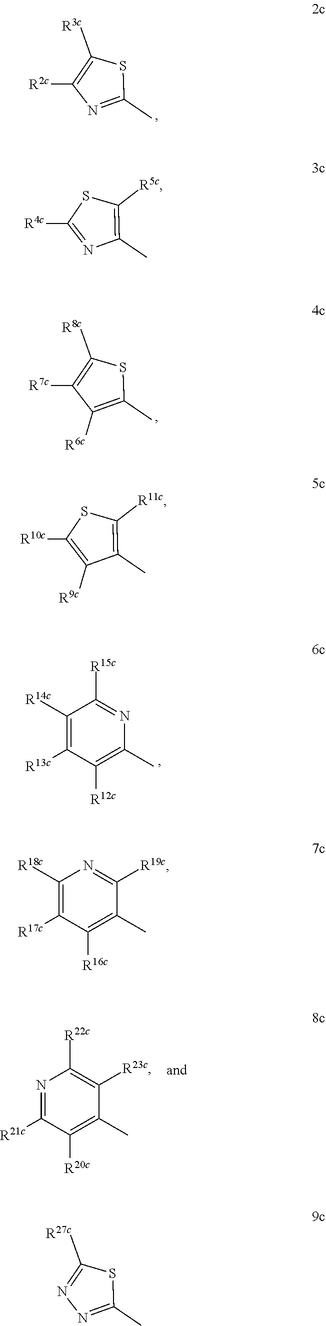
wherein R^{2c} to R^{23c} and R^{27c} are independently selected from H; halogen; OH; CN; SH; S—C₁₋₃ alkyl; C₁₋₃ alkyl, optionally substituted with a F; cyclopropyl, optionally substituted with a F; iso-propyl, optionally substituted with a F; O-cyclopropyl optionally substituted with a F; O-isopropyl optionally substituted with a F; OC₁₋₃ alkyl optionally substituted with a F; NR^{24c}R^{25c}, wherein R^{24c} is selected from H, and C₁₋₃ alkyl, and R^{25c} is selected from H, C₁₋₃ alkyl, and COR^{26c}, wherein R^{26c} is selected from H, and C₁₋₃ alkyl;
X¹ is selected from S, SO, SO₂;
B² is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{27#}—CONH— wherein R^{27#} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{28c}—CONH— wherein R^{28c} is selected from C₁₋₃ alkyl and cyclopropyl; b) an aryl, such as phenyl or naphthyl, optionally substituted with a group selected from a halogen; CN; —COOH; —CONR^{29c}R^{30c}, wherein R^{29c} and R^{39c} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{31c}R^{32c}, wherein R^{31c} and R^{32c} are independently selected from H, C₁₋₃ alkyl and isopropyl; OH; and R^{33c}—CONH—, wherein R^{33c} is selected from C₁₋₃ alkyl and cyclopropyl; c) a C₅₋₇ cycloalkyl, optionally substituted with a substituent selected from a halogen, CN, methyl optionally substituted with a F, OCH₃ optionally substituted with a F, OCH₂CH₃ optionally substituted with a F, OH, and R^{34c}—CONH— wherein R^{34c} is selected from C₁₋₃ alkyl and cyclopropyl; and d) a heterocycle, such as heteroaryl or heterocycloalkyl, optionally substituted with a group selected from a halogen; CN; —COOH; —CONR^{35c}R^{36c}, wherein R^{35c} and R^{36c} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR^{37c}R^{38c}, wherein R^{37c} and R^{38c} are independently selected from H, C₁₋₃ alkyl and isopropyl; OH; and R^{39c}—CONH— wherein R^{39c} is selected from C₁₋₃ alkyl and cyclopropyl; e) a C₁₋₆ alkyl or branched C₃₋₆ alkyl;
R¹ is selected from the group consisting of hydrogen and an in vivo metabolizable group;
R² is selected from the group consisting of hydrogen and an in vivo metabolizable group; and
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³.

38. The prodrug compound of claim 26 having formula II 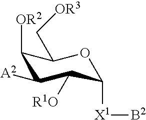
wherein the pyranose ring is α-D-galactopyranose,
A² is 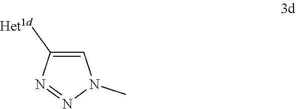
wherein the pyranose ring is α-D-galactopyranose,
Het^{1d} is selected from the group consisting of 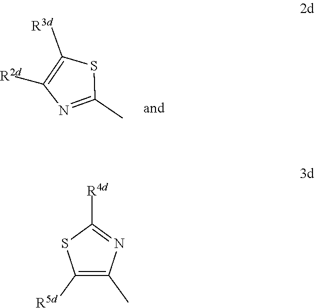
wherein R^{2d} is selected from the group consisting of OH and halogen;
R^{3d} is selected from the group consisting of hydrogen, C₁₋₆ alkyl and halogen;
R^{4d} is selected from the group consisting of OH and halogen;
R^{5d} is selected from the group consisting of hydrogen, C₁₋₆ alkyl and halogen;
X¹ is S;
B² is selected from a) an aryl, such as phenyl or naphthyl, optionally substituted with a group selected from a halogen; CN; —COOH; —CONR^{29d}R^{30d}, wherein R^{29d} and R^{31d} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; SC₁₋₃ alkyl, optionally substituted with a F; O-cyclopropyl, optionally substituted with a F; O-isopropyl, optionally substituted with a F; NR^{31d}R^{32d}, wherein R^{31d} and R^{32d} are independently selected from H, C₁₋₃ alkyl and isopropyl; OH; and R^{33d}—CONH—, wherein R^{33d} is selected from C₁₋₃ alkyl and cyclopropyl; b) a heterocycle, such as heteroaryl or heterocycloalkyl, optionally substituted with a group selected from a halogen; CN; —COOH; —CONR^{35d}R^{36d}, wherein R^{35d} and R^{36d} are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; SC₁₋₃ alkyl, optionally substituted with a F; O-isopropyl, optionally substituted with a F; NR^{37d}R^{38d}, wherein R^{37d} and R^{38d} are independently selected from H, C₁₋₃ alkyl and isopropyl; OH; and R^{39d}—CONH— wherein R^{39d} is selected from C₁₋₃ alkyl and cyclopropyl;
R¹ is selected from the group consisting of hydrogen and an in vivo metabolizable group;
R² is selected from the group consisting of hydrogen and an in vivo metabolizable group; and
R³ is selected from the group consisting of hydrogen and an in vivo metabolizable group; or
a pharmaceutically acceptable salt or solvate thereof;
with the proviso that at least one in vivo metabolizable group is present in at least one from the group consisting of R¹, R² and R³.

39. The prodrug compound of claim 26, wherein:
R¹ is selected from the group consisting of hydrogen and an in vivo metabolizable group;
R² is selected from the group consisting of hydrogen and an in vivo metabolizable group;
R³ is selected from the group consisting of hydrogen and an in vivo metabolizable group;
with the proviso that one in vivo metabolizable group is present in one from the group consisting of R¹, R² and R³.

40. The prodrug of claim 39, wherein R¹ is an in vivo metabolizable group;
R² is hydrogen; and
R³ is hydrogen.

41. The prodrug of claim 39, wherein R² is an in vivo metabolizable group;
R¹ is hydrogen; and
R³ is hydrogen.

42. The prodrug of claim 39, wherein R³ is an in vivo metabolizable group;
R¹ is hydrogen; and
R² is hydrogen.

43. The prodrug compound of claim 39, wherein the in vivo metabolizable group is independently selected from the group consisting of carbamate, ether, phosphate, sulphate, oxy alkyl phosphate, oxy alkyl sulphate, N-Mannich base, carbonate, amide, ester, N-acylsulphoneamides, sulfonamides, imines, acyloxyalkylamines, phosphates, phosphoroimidates, azoconjugates, carbonyloxymethyl, acethylthioethanol, dithioethanol, cyclosal, Hep-direct, phosphorodiimidatesm ProTide phosphoroimidates, Pro Tide phosphonoimidates, alkoxyalkylmonoeters and acetyl.

44. The compound of claim 26 selected from the group consisting of
5-Bromopyridin-3-yl 3-deoxy-3-[4-(3,4,5-trifluorophenyl)-1H-1,2,3-triazol-1-yl]-6-O-phospho-1-thio-α-D-galactopyranoside,
5-Bromopyridin-3-yl 3-deoxy-3-[4-(3,4,5-trifluorophenyl)-1H-1,2,3-triazol-1-yl]-2-O-phospho-1-thio-α-D-galactopyranoside,
3,4-Dichlorphenyl 3-deoxy-3-[4-(3,4,5-trifluorophenyl)-1H-1,2,3-triazol-1-yl]-2-O-phospho-1-thio-α-D-galactopyranoside,
3,4-Dichlorphenyl 3-deoxy-3-[4-(3,4,5-trifluorophenyl)-1H-1,2,3-triazol-1-yl]-2-O-sulfo-1-thio-α-D-galactopyranoside,
5-Bromopyridin-3-yl 3-Deoxy-3-[4-(3,4,5-trifluorophenyl)-1H-1,2,3-triazol-1-yl]-2-O-sulfo-1-thio-α-D-galactopyranoside,
5-Bromopyridin-3-yl 3-Deoxy-3-[4-(3,4,5-trifluorophenyl)-1H-1,2,3-triazol-1-yl]-6-O-sulfo-1-thio-α-D-galactopyranoside,
5-Bromopyridin-3-yl 3-deoxy-3-[4-(3,4,5-trifluorophenyl)-1H-1,2,3-triazol-1-yl]-6-O-[(phosphonooxy)methyl]-1-thio-α-D-galactopyranoside, and
5-Bromopyridin-3-yl 3-deoxy-3-[4-(3,4,5-trifluorophenyl)-1H-1,2,3-triazol-1-yl]-2-O-[(phosphonooxy)methyl]-1-thio-α-D-galactopyranoside, and
5-Bromopyridin-3-yl 3-[4-(4-chloro-3,5-difluorophenyl)-1H-1,2,3-triazol-1-yl]-3-deoxy-2-O-[(phosphonooxy)methyl]-1-thio-α-D-galactopyranoside; or a
pharmaceutically acceptable salt or solvate thereof.

45. A pharmaceutical composition comprising the compound of claim 26 and optionally a pharmaceutically acceptable additive.

46. A method for treatment of a disorder relating to the binding of a galectin-3 to a ligand in a mammal, wherein a therapeutically effective amount of at least one compound according to claim 1 is administered to a mammal in need of said treatment; wherein said disorder is selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistens; obesity; Diastolic HF; asthma and other interstitial lung diseases, including Hermansky-Pudlak syndrome, mesothelioma; liver disorders, such as non-alcoholic steatohepatitis.
